SEQUENCE LISTING (27 sequences)

SEQ ID NO: 1 - protein, 119 aa, Artificial Sequence [sequence omitted]

SEQ ID NO: 2 - protein, 107 aa, Artificial Sequence [sequence omitted]

SEQ ID NO: 3 - protein, 121 aa, Artificial Sequence
  source: /note=“Description of Artificial Sequence Synthetic polypeptide”
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 4 - protein, 107 aa, Artificial Sequence
  source: /note=“Description of Artificial Sequence Synthetic polypeptide”
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 5 - protein, 120 aa, Artificial Sequence
  source: /note=“Description of Artificial Sequence Synthetic polypeptide”
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 6 - protein, 112 aa, Artificial Sequence
  source: /note=“Description of Artificial Sequence Synthetic polypeptide”
DVVMTQSPLSLPVTLGQPASISCRSSQSLLHSSGNTYLEYFQQRPGQSPRPLIYKISTRFSGVPDRFSGSGSGTDFTLKISRVEAEDVGVYYCFQGSHVPYTFGGGTKVEIK

SEQ ID NO: 7 - protein, 120 aa, Artificial Sequence [sequence omitted]

SEQ ID NO: 8 - protein, 107 aa, Artificial Sequence [sequence omitted]

SEQ ID NO: 9 - protein, 123 aa, Artificial Sequence
  source: /note=“Description of Artificial Sequence Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 10 - protein, 106 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 11 - protein, 118 aa, Artificial Sequence [sequence omitted]

SEQ ID NO: 12 - protein, 107 aa, Artificial Sequence [sequence omitted]

SEQ ID NO: 13 - protein, 123 aa, Artificial Sequence
  source: /note=“Description of Artificial Sequence Synthetic polypeptide”
EVKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSS

SEQ ID NO: 14 - protein, 112 aa, Artificial Sequence
  source: /note=“Description of Artificial Sequence Synthetic polypeptide”
DIELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEVK

SEQ ID NO: 15 - protein, 123 aa, Artificial Sequence
  source: /note=“Description of Artificial Sequence Synthetic polypeptide”
EVKLEESGGGFVKPGGSLKISCAASGFTFRNYAMSWVRLSPEMRLEWVATISSAGGYIFYSDSVQGRFTISRDNAKNTLHLQMGSLRSGDTAMYYCARQGFGNYGDYYAMDYWGQGTTVTVSS

SEQ ID NO: 16 - protein, 112 aa, Artificial Sequence
  source: /note=“Description of Artificial Sequence Synthetic polypeptide”
DIELTQSPSSLAVSAGEKVTMSCKSSQSLLNSRTRKNQLAWYQQKTGQSPELLIYWASTRQSGVPDRFTGSGSGTDFTLTISSVQAEDLAVYYCQQSYNLLTFGPGTKLEIK

SEQ ID NO: 17 - protein, 120 aa, Artificial Sequence
  source: /note=“Description of Artificial Sequence Synthetic polypeptide”
QVQLVQSGAEVKKPGASVKVSCKASGYTIEDYYMHWVRQAPGQGLEWMGWIDPENGDTEYAPTFQGRVTMTRDTSINTAYMELSRLRSDDTAVYYCARHDAHYGTWFAYWGQGTLVTVSS

SEQ ID NO: 18 - protein, 112 aa, Artificial Sequence
  source: /note=“Description of Artificial Sequence Synthetic polypeptide”
DVVMTQSPLSLPVTLGQPASISCRSSQSIIRNDGNTYLEWYQQRPGQSPRRLIYRVSNRFSGVPDRFSGSGSGTDFTLKISRVEAEDVGVYYCFQGSHVPYTFGGGTKVEIK

SEQ ID NO: 19 - protein, 117 aa, Artificial Sequence
  source: /note=“Description of Artificial Sequence Synthetic polypeptide”
EIQLQQSGPELMKPGASVKISCKASTYSFTRYFMHWVKQSHGESLEWIGYIDPFNGGTGYNQKFKGKATLTVDKSSSTAYMHLSSLTSEDSAVYYCVTYGSDYFDYWGQGTTLTVSS

SEQ ID NO: 20 - protein, 108 aa, Artificial Sequence
  source: /note=“Description of Artificial Sequence Synthetic polypeptide”
DIVMTQPQKFMSTSVGDRVSVTCKASQNVETDVVWYQQKPGQPPKALIYSASYRHSGVPDRFTGSGSGTNFTLTISTVQSEDLAEYFCQQYNNYPFTFGSGTKLEIIR

SEQ ID NO: 21 - protein, 120 aa, Artificial Sequence; identical to SEQ ID NO: 5
  source: /note=“Description of Artificial Sequence Synthetic polypeptide”

SEQ ID NO: 22 - protein, 112 aa, Artificial Sequence
  source: /note=“Description of Artificial Sequence Synthetic polypeptide”
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIK

SEQ ID NO: 23 - protein, 116 aa, Artificial Sequence
  source: /note=“Description of Artificial Sequence Synthetic polypeptide”
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 24 - protein, 107 aa, Artificial Sequence
  source: /note=“Description of Artificial Sequence Synthetic polypeptide”
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 25 - protein, 5 aa, Unknown
  source: /note=“Description of Unknown recognition motif sequence”
  MOD_RES (3)..(3): Any amino acid
LPXTG

SEQ ID NO: 26 - protein, 4 aa, Artificial Sequence
  source: /note=“Description of Artificial Sequence Synthetic peptide”
GFLG

SEQ ID NO: 27 - protein, 4 aa, Artificial Sequence
  source: /note=“Description of Artificial Sequence Synthetic peptide”
ALAL

(19) United States
(12) Patent Application Publication
(10) Pub. No.: US 2022/0323596 A1
(43) Pub. Date: Oct. 13, 2022

(54) ANTIBODY CONJUGATES OF TOLL-LIKE RECEPTOR AGONISTS

(71) Applicant: SILVERBACK THERAPEUTICS, INC., Seattle, WA (US)

(72) Inventors: Sean Wesley SMITH, Seattle, WA (US); Craig Alan COBURN, Seattle, WA (US)

(21) Appl. No.: 17/275,542

(22) PCT Filed: Sep. 12, 2019
(86) PCT No.: PCT/US2019/050892

§ 371 Date: Mar. 11, 2021

Related U.S. Application Data
(60) Provisional application No. 62/730,466, filed on Sep. 12, 2018.

(51) Int. Cl. A61K 47/68; C07D 471/04; A61K 47/54; A61P 35/00; A61P 1/00

(52) U.S. Cl. CPC A61K 47/6803; C07D 471/04; A61K 47/545; A61K 47/6855; A61P 35/00; A61P 1/00

(57) ABSTRACT

Imidazoquinoline compounds of formula (IA), conjugates, and pharmaceutical compositions for use in the treatment of disease, such as cancer, are disclosed herein. The disclosed imidazoquinoline compounds are useful, among other things, in the treating of cancer and modulating TLR7. Additionally, imidazoquinoline compounds incorporated into a conjugate with an antibody construct are described herein.

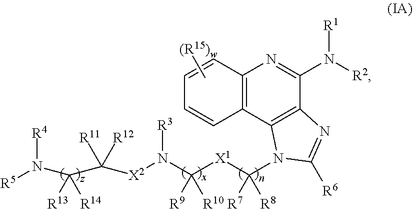

### RELATED APPLICATION INFORMATION

This application is a U.S. National Stage Application which claims priority to PCT Application No. PCT/US2019/050892, filed Sep. 12, 2019, which claims the benefit of U.S. Provisional Application No. 62/730,466 filed Sep. 12, 2018, the content of which is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is SEQUENCE_LISTING_860234_426USPC. The text file is 27.9 KB, was created on Mar. 11, 2021, and is being submitted electronically via EFS-Web.

### BACKGROUND

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

### SUMMARY

In some aspects, the present disclosure provides a compound represented by Formula (IA):

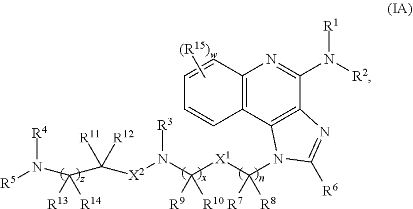

or a salt thereof, wherein:

R¹, R², R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R¹, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen; R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In some embodiments, X¹ is O. In some embodiments, n is 2. In some embodiments, x is 2. In some embodiments, z is 0. In some embodiments, z is 1. In some embodiments, the compound of Formula (IA) is represented by Formula (IB):

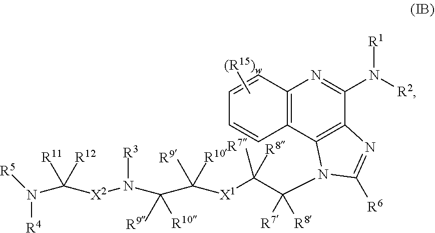

or a salt thereof, wherein:

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In some embodiments, the compound of Formula (IA) is represented by Formula (IC):

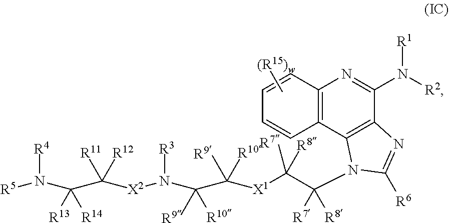

or a salt thereof, wherein.

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen. In some embodiments, R¹, R², R³, R⁴, and R⁵ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In some embodiments, R¹ and R² are independently selected from hydrogen and C₁₋₆ alkyl. In some embodiments, R¹ and R² are each hydrogen. In some embodiments, R³ is selected from hydrogen and C₁₋₆ alkyl optionally substituted with one or more halogens. In some embodiments, R³ is hydrogen. In some embodiments, R⁴ is selected from hydrogen and C₁₋₆ alkyl optionally substituted with one or more halogens. In some embodiments, R⁴ is hydrogen. In some embodiments, R⁵ is selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In some embodiments, R⁵ is hydrogen. In some embodiments, R⁶ is selected from halogen, —OR²⁰, and —N(R²⁰)₂; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle. In some embodiments, R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle. In some embodiments, R⁶ is C₁₋₆ alkyl substituted with —OR²⁰, and R²⁰ is selected from hydrogen and C₁₋₆ alkyl optionally substituted with one or more substituents independently selected from halogen, —OH, and —NH2. In some embodiments, R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen. In some embodiments, R^{7′} and R^{8′} are each hydrogen. In some embodiments, R^{7″} and R^{8″} are each C₁₋₆ alkyl. In some embodiments, R^{7″} and R^{8″} are each methyl. In some embodiments, R^{9′}, R^{9″}, R^{10′}, and R^{10″} are independently selected at each occurrence from hydrogen and C₁₋₆ alkyl. In some embodiments, R^{9′}, R^{9″}, R^{10′}, and R^{10″} are each hydrogen. In some embodiments, R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, —OC(O)R²⁰; and C₁₋₆ alkyl, optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In some embodiments, R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In some embodiments, R³ and R¹¹ taken together form an optionally substituted 5- to 6-membered heterocycle. In some embodiments, R¹¹ and R¹² taken together form an optionally substituted C₃₋₆ carbocycle. In some embodiments, X² is C(O). In some embodiments, a compound of the current disclosure is represented by the structure:

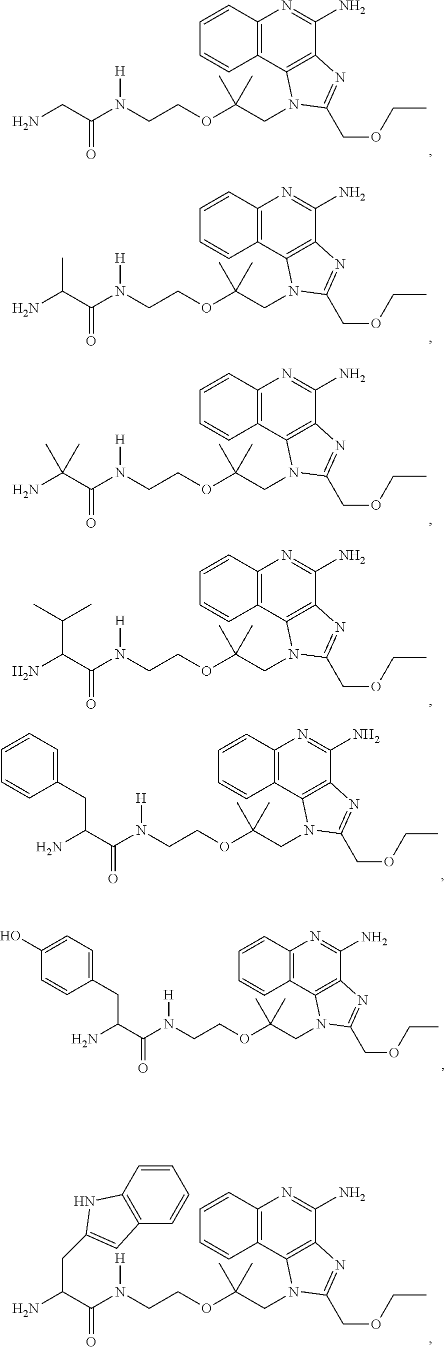

or a salt of any one thereof.

In some aspects, the present disclosure provides a pharmaceutical composition comprising a compound or salt disclosed herein, and a pharmaceutically acceptable excipient. In some embodiments, the compound or salt is further covalently bound to a linker, L³.

In some aspects, the present disclosure provides a compound represented by Formula (IIA):

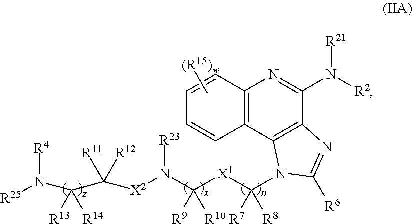

or a salt thereof, wherein:

R² and R⁴ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²¹, R²³, and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen; R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, —CN, C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

L³ is a linker;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In some embodiments, X¹ is O. In some embodiments, n is 2. In some embodiments, x is 2. In some embodiments, z is 0. In some embodiments, z is 1. In some embodiments, the compound of Formula (IIA) is represented by (IIB) or (IIC):

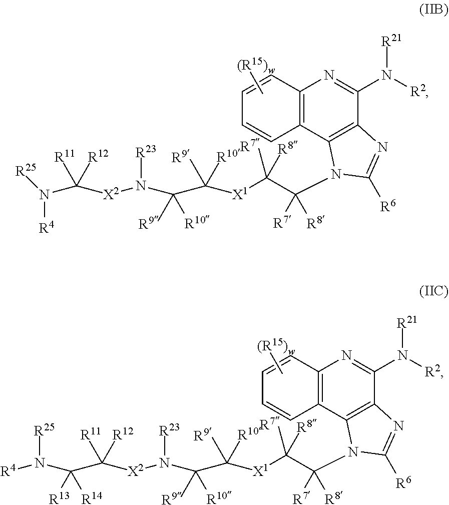

or a salt thereof, wherein.

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In some embodiments, R² and R⁴ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In some embodiments, R² and R⁴ are independently selected from hydrogen and C₁₋₆ alkyl. In some embodiments, R² and R⁴ are each hydrogen. In some embodiments, R²³ is selected from hydrogen and C₁₋₆ alkyl optionally substituted with one or more halogens. In some embodiments, R²³ is hydrogen. In some embodiments, R²¹ is selected from hydrogen and C₁₋₆ alkyl optionally substituted with one or more halogens. In some embodiments, R²¹ is hydrogen. In some embodiments, R²¹ is L³. In some embodiments, R²⁵ is selected from hydrogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In some embodiments, R²⁵ is hydrogen. In some embodiments, R²⁵ is L³. In some embodiments, R⁶ is selected from halogen, —OR²⁰, and —N(R²⁰)₂; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle. In some embodiments, R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and R²⁰ is independently selected at each occurrence from hydrogen, —NH₂, —C(O)OCH₂C₆H₅; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle. In some embodiments, R⁶ is C₁₋₆ alkyl substituted with —OR²⁰, and R²⁰ is selected from hydrogen and C₁₋₆ alkyl, which is optionally substituted with one or more substituents independently selected from halogen, —OH, and —NH₂. In some embodiments, R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl optionally substituted with one or more substituents independently selected from halogen. In some embodiments, R^{7′} and R^{8′} are hydrogen. In some embodiments, R^{7″} and R^{8″} are C₁₋₆ alkyl. In some embodiments, R^{7″} and R^{8″} are methyl. In some embodiments, R^{9′}, R^{9″}, R^{10′}, and R^{10″} are independently selected at each occurrence from hydrogen and C₁₋₆ alkyl. In some embodiments, R^{9′}, R^{9″}, R^{10′}, and R^{10″} are each hydrogen. In some embodiments, R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In some embodiments, R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In some embodiments, R²³ and R¹¹ taken together form an optionally substituted 5- to 6-membered heterocycle. In some embodiments, R¹¹ and R¹² taken together form an optionally substituted C₃₋₆ carbocycle. In some embodiments, X² is C(O). In some embodiments, L³ is a cleavable linker. In some embodiments, L³ is cleavable by a lysosomal enzyme. In some embodiments, L³ is represented by the formula:

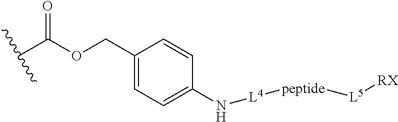

wherein:

L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and

R³⁰ is independently selected at each occurrence from halogen, —OH, —CN, —O-alkyl, —SH, ═O, ═S, —NH₂, —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂.

In some embodiments, RX comprises a leaving group. In some embodiments, RX is a maleimide or an alpha-halo carbonyl. In some embodiments, the peptide of L³ comprises Val-Cit or Val-Ala. In some embodiments, L³ is represented by the formula:

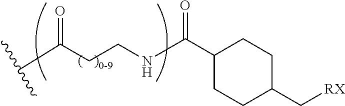

wherein:

RX comprises a reactive moiety; and

n is 0-9.

In some embodiments, RX comprises a leaving group. In some embodiments, RX is a maleimide or an alpha-halo carbonyl. In some embodiments, L³ is further covalently bound to an antibody construct to form a conjugate.

In some aspects, the present disclosure provides a conjugate represented by the formula:

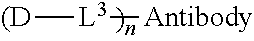

wherein:

Antibody is an antibody construct;

n is 1 to 20;

D is the compound or salt disclosed herein; and

L³ is a linker moiety.

In some embodiments, n is selected from 1 to 8. In some embodiments, n is selected from 2 to 5. In some embodiments, n is 2. In some embodiments, -L³ is represented by the formula:

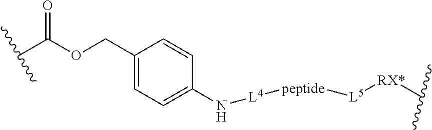

wherein:

L⁴ represents the C-terminus of the peptide and L⁵ is selected from a bond, alkylene and heteroalkylene, wherein L⁵ is optionally substituted with one or more groups independently selected from R³⁰; RX* is a bond, a succinimide moiety, or a hydrolyzed succinimide moiety bound to a residue of an antibody construct, wherein [symbol] on RX* represents the point of attachment to the residue of the antibody construct; and

R³⁰ is independently selected at each occurrence from halogen, —OH, —CN, —O-alkyl, —SH, ═O, ═S, —NH₂, —NO₂; and C₁-C₁₀alkyl, C₂-C₁₀ alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

In some embodiments, RX* is a succinamide moiety, hydrolyzed succinamide moiety or a mixture thereof and is bound to a cysteine residue of an antibody construct. In some embodiments, -L³ is represented by the formula:

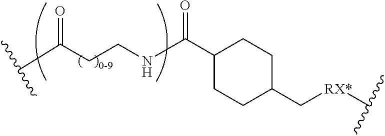

wherein:

RX* is a bond, a succinimide moiety, or a hydrolyzed succinimide moiety bound to a residue of an antibody construct, wherein [symbol] on RX* represents the point of attachment to the residue of the antibody construct; and

n is 0-9.

In some embodiments, the antibody construct comprises an antigen binding domain specifically binds to an antigen selected from the group consisting of CD5, CD19, CD20, CD25, CD37, CD30, CD33, CD40, CD45, CAMPATH-1, BCMA, CS-1, PD-L1, B7-H₃, B7-DC, HLD-DR, carcinoembryonic antigen (CEA), TAG-72, EpCAM, MUC1, folate-binding protein, A33, G250, prostate-specific membrane antigen (PSMA), GD2, GD3, GM2, Le^{y}, CA-125, CA19-9, epidermal growth factor, p185HER2, IL-2 receptor, EGFRvIII (de2-7 EGFR), fibroblast activation protein, tenascin, a metalloproteinase, endosialin, vascular endothelial growth factor, avB3, WT1, LMP2, HPV E6, HPV E7, Her-2/neu, MAGE A3, p53 nonmutant, NY-ESO-1, MelanA/MART1, Ras mutant, gp100, p53 mutant, PR1, ber-abl, tyronsinase, survivin, PSA, hTERT, a Sarcoma translocation breakpoint protein, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TESL Sperm protein 17, LCK, HMWMAA, AKAP-4, SSX2, XAGE 1, Legumain, Tie 3, VEGFR2, MAD-CT-1, PDGFR-B, MAD-CT-2, ROR2, TRAIL1, MUC16, MAGE A4, MAGE C2, GAGE, EGFR, CMET, HER3, MUC15, CA6, NAPI2B, TROP2, CLDN6, CLDN16, CLDN18.2, CLorfl86, RON, LY6E, FRA, DLL3, PTK7, STRA6, TMPRSS3, TMPRSS4, TMEM238, UPK1B, VTCN1, LIV1, ROR1, or Fos-related antigen 1, preferably Her2.

In some aspects, the present disclosure provides a pharmaceutical composition, comprising a conjugate disclosed herein, and a pharmaceutically acceptable excipient. In some embodiments, the average Drug-to-Antibody Ratio (DAR) is 1 to 8.

In some aspects, the present disclosure provides a method for the treatment of cancer, comprising administering an effective amount of a compound or salt disclosed herein or a pharmaceutical composition disclosed herein to a subject in need thereof.

In some aspects, the present disclosure provides a method of killing tumor cells in vivo, comprising contacting a tumor cell population with a conjugate disclosed herein or a pharmaceutical composition disclosed herein. In some aspects, the present disclosure provides a method for treatment, comprising administering to a subject a conjugate or a pharmaceutical composition disclosed herein.

In some aspects, the present disclosure provides a compound or salt or a pharmaceutical composition for use in a method of treatment of a subject's body by therapy. In some aspects, the present disclosure provides a compound or salt or a pharmaceutical composition disclosed herein for use in a method of treating cancer. In some aspects, the present disclosure provides a conjugate disclosed herein or a pharmaceutical composition disclosed herein for use in a method of treatment of a subject's body by therapy. In some aspects, the present disclosure provides a conjugate disclosed herein or a pharmaceutical composition disclosed herein for use in a method of treating cancer.

In some aspects, the present disclosure provides a method of preparing an antibody conjugate of the formula:

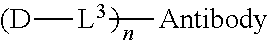

wherein:

Antibody is an antibody construct;

n is selected from 1 to 20; and

D-L³ is selected from a compound or salt disclosed herein, comprising contacting D-L³ with an antibody construct.

In some aspects, the present disclosure provides a method of preparing an antibody conjugate of the formula:

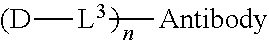

wherein:

Antibody is an antibody construct;

n is selected from 1 to 20;

L³ is a linker; and

D is selected from a compound or salt disclosed herein,

In some embodiments, the method comprises contacting L³ with the antibody construct to form L³-antibody and contacting L³ antibody with D to form the conjugate. In some embodiments, the antibody construct comprises an antigen binding domain specifically binds to an antigen selected from the group consisting of CD5, CD19, CD20, CD25, CD37, CD30, CD33, CD40, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), GD2, GD3, GM2, Le^{y}, CA-125, CA19-9, epidermal growth factor, p185HER2, IL-2 receptor, EGFRvIII (de2-7 EGFR), fibroblast activation protein, tenascin, a metalloproteinase, endosialin, vascular endothelial growth factor, avB3, WT1, LMP2, HPV E6, HPV E7, Her-2/neu, MAGE A3, p53 nonmutant, NY-ESO-1, MelanA/MART1, Ras mutant, gp100, p53 mutant, PR1, bcr-abl, tyronsinase, survivin, PSA, hTERT, a Sarcoma translocation breakpoint protein, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TESL Sperm protein 17, LCK, HMWMAA, AKAP-4, SSX2, XAGE 1, Legumain, Tie 3, VEGFR2, MAD-CT-1, PDGFR-B, MAD-CT-2, ROR2, TRAIL1, MUC16, MAGE A4, MAGE C2, GAGE, EGFR, CMET, HER3, MUC15, CA6, NAPI2B, TROP2, CLDN6, CLDN16, CLDN18.2, CLorfl86, RON, LY6E, FRA, DLL3, PTK7, STRA6, TMPRSS3, TMPRSS4, TMEM238, UPK1B, VTCN1, LIV1, ROR1, or Fos-related antigen 1, preferably Her2. In some embodiments, the method further comprises purifying the antibody conjugate.

### BRIEF DESCRIPTION OF THE DRAWINGS

FIG. 1 shows in vitro TLR7 small molecule screening.

FIG. 2 shows in vitro Her2/TLR7 immune-stimulatory conjugate screening.

FIG. 3 shows in vitro Her2/TLR7 immune-stimulatory conjugate screening.

FIG. 4A-FIG. 4F show treatment with anti-Her2-TLR7 agonist conjugate inhibits tumor growth in CT26-Her2 bearing mice.

FIG. 5 shows treatment with anti-Her2-TLR7 agonist conjugate improves survival of CT26-Her2 bearing mice.

FIG. 6A shows treatment with anti-HER2-TLR7 agonist conjugate slows tumor growth in HER2+ EMT6 cell-bearing mice.

FIG. 6B shows treatment with anti-HER2-TLR7 agonist conjugate improves survival of HER2+ EMT6 cell-bearing mice.

FIG. 7A shows treatment with 5 mg/kg anti-HER2-TLR7 conjugate confers anti-tumor memory response on mice re-challenged with HER2+CT26 tumors.

FIG. 7B shows treatment with 20 mg/kg anti-HER2-TLR7 conjugate confers anti-tumor memory response on mice re-challenged with HER2+CT26 tumors.

FIG. 8 shows treatment with anti-HER2-TLR7 conjugate protects re-challenged mice from growth of wild-type CT26 tumor cells.

FIG. 9A shows anti-HER2-TLR7 conjugate activates mouse bone marrow cells when bound to HER2-positive SK-BR-3 tumor cells.

FIG. 9B shows anti-HER2-TLR7 conjugate does not activate mouse bone marrow cells when unbound in the presence of HER2-negative MDA-MB-468 tumor cells.

FIG. 10A shows treatment with a single dose of anti-HER2-TLR7 conjugate increases intratumoral levels of chemokines and cytokines in HER2+CT26 tumor-bearing mice.

FIG. 10B shows treatment with three doses of anti-HER2-TLR7 conjugate increases intratumoral levels of chemokines and cytokines in HER2+CT26 tumor-bearing mice.

FIG. 11A shows treatment with anti-HER2-TLR7 conjugate increases the percentage of AH-1 tetramer-positive T-cells in HER2+CT26 tumor-bearing mice.

FIG. 11B shows treatment with anti-HER2-TLR7 conjugate increases M1 to M2 phenotype ratio in macrophages from HER2+CT26 tumor-bearing mice.

FIG. 11C shows treatment with anti-HER2-TLR7 conjugate increases the percentage of CD8-positive T-cells that are also IFNγ and TNFα in HER2+CT26 tumor-bearing mice.

FIG. 11D shows treatement with a single dose of anti-HER2-TLR7 conjugate increases the surface expression of PD-L1 in tumor cells of HER2+CT26 tumor-bearing mice.

FIG. 11E shows treatment with three doses of anti-HER2-TLR7 conjugate increases the surface expression of PD-L1 in tumor cells of HER2+CT26 tumor-bearing mice.

FIG. 11F shows treatment with a single dose of anti-HER2-TLR7 conjugate increases the percentage of CD45+/Gr1+/CD11b+ cells in tumors of HER2+CT26 tumor-bearing mice.

FIG. 11G shows treatment with three doses of anti-HER-TLR7 conjugate increases the percentage of CD45+/Gr1+/CD11b+ cells in tumors of HER2+CT26 tumor-bearing mice.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

While preferred embodiments of the present invention have been shown and described herein, it will be evident to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present disclosure provides compounds, conjugates and pharmaceutical compositions for use in the treatment of disease. In certain embodiments, the compounds of the disclosure are TLR7 modulators. In certain embodiments, the compounds are TLR7 agonists. Toll-like receptors (TLRs) are a family of membrane-spanning receptors that are expressed on cells of the immune system like dendritic cells, macrophages, monocytes, T cells, B cells, NK cells and mast cells but also on a variety of non-immune cells such as endothelial cells, epithelial cells and even tumor cells. TLRs can have many isoforms, including TLR4, TLR7 and TLR8.

TLR7 receptors play a role in pathogen recognition and activation of innate immunity. They recognize certain pathogen-associated molecular patterns (PAMPs) that are expressed on infectious agents, and mediate the production of cytokines necessary for the development of effective immunity. TLR7 is a nucleotide-sensing TLR which is activated by single-stranded RNA. The gene encoding TRL7 is predominantly expressed in lung, placenta, and spleen.

Several agonists targeting activation of different TLRs can be used in various immunotherapies, including vaccine adjuvants and in cancer immunotherapies. TLR agonists can range from simple molecules to complex macromolecules. Likewise, the sizes of TLR agonists can range from small to large. TLR agonists can be synthetic or biosynthetic agonists. TLR agonists can also be Pathogen-Associated Molecular Pattern molecules (PAMPs).

The compounds of the present disclosure may be useful for the treatment and prevention, e.g., vaccination, of cancer, autoimmune diseases, inflammation, sepsis, allergy, asthma, graft rejection, graft-versus-host disease, immunodeficiencies, and infectious diseases.

In certain embodiments, the compounds have utility in the treatment of cancer either as single agents or in combination therapy. In certain embodiments, the compounds have utility as single agent immunomodulators, vaccine adjuvants and in combination with conventional cancer therapies. In certain embodiments, the compounds are incorporated into a conjugate that can be utilized, for example, to enhance an immune response. In certain embodiments, the disclosure provides antibody construct-imidazoquinoline compound conjugates (also referred to herein as antibody construct conjugates or conjugates) and their use for treating cancer.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

As used herein, an “amine masking group” refers to any moiety covalently bound to the nitrogen of an amine, e.g., primary amine, which attenuates the interaction or activity, or blocks the amine from interacting with a TLR7 receptor and that is removable from the amine. Examples of amine masking groups include enzymatically-cleavable promoieties such as amino acids or peptides.

As used herein, the term “antibody” refers to an immunoglobulin molecule that specifically binds to, or is immunologically reactive toward, a specific antigen. The term antibody can include, for example, polyclonal, monoclonal, genetically engineered, and antigen binding fragments thereof. An antibody can be, for example, murine, chimeric, humanized, heteroconjugate, bispecific, a diabody, a triabody, or a tetrabody. The antigen binding fragment can include, for example, Fab′, F(ab′)₂, Fab, Fv, rIgG, and scFv.

As used herein, an “antigen binding domain” refers to a region of a molecule that specifically binds to an antigen. An antigen binding domain may be a domain that can specifically bind to an antigen. An antigen binding domain can be an antigen-binding portion of an antibody or an antibody fragment. An antigen binding domain can be one or more fragments of an antibody that retain the ability to specifically bind to an antigen. An antigen binding domain can be an antigen binding fragment. An antigen binding domain can recognize a single antigen. In some embodiments, an antigen binding domain can recognize, for example, more than one antigen.

As used herein, an “antibody construct” refers to a molecule, e.g., a protein, peptide, antibody or portion thereof, that contains an antigen binding domain and an Fc domain. An antibody construct can recognize, for example, multiple antigens.

As used herein, the abbreviations for amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Other amino acids include citrulline (Cit); homocysteine (Hey); hydroxyproline (Hyp); ornithine (Orn); and thyroxine (Thx).

“Conjugate”, as used herein, refers to an antibody construct that is linked, i.e., covalently linked, either directly or through a linker to a compound or compound-linker described herein, e.g., a compound or salt of any one of Formulas (IA), (IB), (IC), (VIIA), (VIIB), or (VIIC) or Formulas (IIA), (IIB), (IIC), (VIIIA), (VIIIB), or (VIIIC), respectively

As used herein, an “Fc domain” can be an Fc domain from an antibody or from a non-antibody that can bind to an Fc receptor.

As used herein, “recognize” with regard to antibody interactions refers to the specific association or specific binding between an antigen binding domain or portion thereof and an antigen.

As used herein, a “target binding domain” refers to a construct that contains an antigen binding domain from an antibody or from a non-antibody that can bind to the antigen.

As used herein, a “tumor antigen” is an antigenic substance associated with a tumor or cancer cell, and can trigger an immune response in a host.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene-refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atoms (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atoms (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkylene” refers to a divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more carbons are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., an NH or NH2 of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R b-C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrase “targeting moiety” refers to a structure that has a selective affinity for a target molecule relative to other non-target molecules. The targeting moiety binds to a target molecule. A targeting moiety may include, for example, an antibody, a peptide, a ligand, a receptor, or a binding portion thereof. The target biological molecule may be a biological receptor or other structure of a cell such as a tumor antigen.

### Antibody Construct

Disclosed herein are antibody constructs that may be used together with compounds of the disclosure. In certain embodiments, compounds of the disclosure are linked, e.g., covalently linked, either directly or through a linker to a compound of the disclosure forming conjugates. In certain embodiments, conjugates of the disclosure are represented by the following formula:

A [symbol] L³-D)ₙ,

wherein A is an antibody construct, L³ is a linker, D is a compound or salt of any one of Formulas (IA), (IB), (IC), (VIIA), (VIIB), or (VIIC), or L³-D is a compound or salt of any one of Formulas (IIA), (IIB), (IIC), (VIIIA), (VIIIB) or (VIIIC), and n is from 1 to 20. In certain embodiments, n is from 1 to 10, such as from 1 to 9, such as from 1 to 8, such as from 2 to 8, such as from 1 to 6, such as from 3 to 5, or such as from 1 to 3. In certain embodiments, n is 4. In certain embodiments, n is 2. In certain embodiments, each D or L³-D are independently selected from Formulas (IA), (IB), (IC), (VIIA), (VIIB), or (VIIC), or Formulas (IIA), (IIB), (IIC), (VIIIA), (VIIIB) or (VIIIC), respectively.

In certain embodiments, a compound or salt of the disclosure, e.g., a compound or salt of Formula (IA), (IB), (IC), (VIIA), (VIIB), or (VIIC), may be referred to herein as a drug, D, an imidazoquinoline compound, an immune-stimulatory compound, an ISC, or a payload, particularly when referenced as part of a conjugate. “LP”, “linker-payload”, “L³-D”, or “compound-linker” may be used herein to refer to a compound or salt of the disclosure bound to a linker.

An antibody construct may contain, for example, two, three, four, five, six, seven, eight, nine, ten, or more antigen binding domains. An antibody construct may contain two antigen binding domains in which each antigen binding domain can recognize the same antigen. An antibody construct may contain two antigen binding domains in which each antigen binding domain can recognize different antigens. An antigen binding domain may be in a scaffold, in which a scaffold is a supporting framework for the antigen binding domain. An antigen binding domain may be in a non-antibody scaffold. An antigen binding domain may be in an antibody scaffold. An antibody construct may comprise an antigen binding domain in a scaffold. The antibody construct may comprise a Fc fusion protein. In some embodiments, the antibody construct is a Fc fusion protein. An antigen binding domain may specifically bind to a tumor antigen. An antigen binding domain may specifically bind to an antigen that is at least 80%, at least 90%, at least 95%, at least 99%, or 100% homologous to a tumor antigen. An antigen binding domain may specifically bind to an antigen on an antigen presenting cell (APC). An antigen binding domain may specifically bind to an antigen that is at least 80%, at least 90%, at least 95%, at least 99%, or 100% homologous to an antigen on an antigen presenting cell (APC).

An antigen binding domain of an antibody may comprise one or more light chain (LC) CDRs and one or more heavy chain (HC) CDRs. For example, an antibody binding domain of an antibody may comprise one or more of the following: a light chain complementary determining region 1 (LC CDR1), a light chain complementary determining region 2 (LC CDR2), or a light chain complementary determining region 3 (LC CDR3). For another example, an antibody binding domain may comprise one or more of the following: a heavy chain complementary determining region 1 (HC CDR1), a heavy chain complementary determining region 2 (HC CDR2), or a heavy chain complementary determining region 3 (HC CDR3). As an additional example, an antibody binding domain of an antibody may comprise one or more of the following: LC CDR1, LC CDR2, LC CDR3, HC CDR1, HC CDR2, and HC CDR3.

The antigen binding domain of an antibody construct may be selected from any domain that binds the antigen including, but not limited to, from a monoclonal antibody, a polyclonal antibody, a recombinant antibody, or an antigen binding fragment thereof, for example, a heavy chain variable domain (V_{H}) and a light chain variable domain (V_{L}), or from a DARPin, an affimer, an avimer, a knottin, a monobody, an affinity clamp, an ectodomain, a receptor ectodomain, a receptor, a T cell receptor, or a recombinant T cell receptor. In certain embodiments, the antigen binding domain of an antibody construct may be selected from any domain that binds the antigen including, but not limited to, from a monoclonal antibody, a polyclonal antibody, a recombinant antibody, or an antigen binding fragment thereof, for example, a heavy chain variable domain (V_{H}) and a light chain variable domain (V_{L}).

The antigen binding domain of an antibody construct may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or to a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor. In certain embodiments, an antigen binding domain of an antibody construct may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}).

In certain embodiments, an antibody construct comprises an Fc domain that may further comprise an Fc domain, in which the Fc domain may be the part of an Fc region that interacts with Fc receptors. The Fe domain of an antibody construct may interact with Fc-receptors (FcRs) found on immune cells. The Fc domain may also mediate the interaction between effector molecules and cells, which can lead to activation of the immune system. The Fc domain may be derived from IgG, IgA, or IgD antibody isotypes, and may comprise two identical protein fragments, which are derived from the second and third constant domains of the antibody's heavy chains. In an Fc domain derived from an IgG antibody isotype, the Fc region may comprise a highly-conserved N-glycosylation site, which may be essential for FcR-mediated downstream effects. The Fc domain may be derived from IgM or IgE antibody isotypes, in which the Fc domain may comprise three heavy chain constant domains.

An Fc domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

An antibody may consist of two identical light protein chains and two identical heavy protein chains, all held together covalently by disulfide linkages. The N-terminal regions of the light and heavy chains together may form the antigen recognition site of an antibody. Structurally, various functions of an antibody may be confined to discrete protein domains. The sites that can recognize and can bind antigen may consist of three complementarities determining regions (CDRs) that may lie within the variable heavy chain region and variable light chain region at the N-terminal end of the heavy chain and the light chain. The constant domains may provide the general framework of the antibody and may not be involved directly in binding the antibody to an antigen, but may be involved in various effector functions, such as participation of the antibody in antibody-dependent cellular cytotoxicity, and may bind Fc receptors. The constant domains may include an Fc region. The constant domains may include an Fc domain. The domains of natural light and heavy chains may have the same general structures, and each domain may comprise four framework regions, whose sequences can be somewhat conserved, connected by three hyper-variable regions or CDRs. The four framework regions (FR) may largely adopt a β-sheet conformation and the CDRs can form loops connecting, and in some aspects forming part of, the β-sheet structure. The CDRs in each chain may be held in close proximity by the framework regions and, with the CDRs from the other chain, may contribute to the formation of the antigen binding site.

An antibody construct may comprise a light chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence. An antibody construct may comprise a heavy chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence.

An antibody of an antibody construct may include an antibody of any type, which may be assigned to different classes of immunoglobins, e.g., IgA, IgD, IgE, IgG, and IgM. Several different classes may be further divided into isotypes, e.g., IgG1, IgG2, IgG3, IgG4, IgA1, and IgA2. An antibody may further comprise a light chain and a heavy chain, often more than one chain. The heavy-chain constant regions (Fc) that corresponds to the different classes of immunoglobulins may be α, δ, ε, γ, and μ, respectively. The light chains may be one of either kappa (κ) or lambda (λ), based on the amino acid sequences of the constant domains. The Fc region may contain an Fc domain. An Fc receptor may bind an Fc domain. Antibody constructs may also include any fragment or recombinant forms thereof, including but not limited to, single chain variable fragments (scFvs), ‘T-bodies’, anti-calins, centyrins, affibodies, domain antibodies, or peptibodies.

An antibody construct may comprise an antibody fragment. An antibody fragment may include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, may be coded for by separate genes, they may be linked by a synthetic linker to be made as a single protein chain in which the V_{L} and V_{H} regions pair to form monovalent molecules.

An antibody may include an Fc region comprising multiple Fe domains. The Fe domain of an antibody may interact with FcRs found on immune cells. The Fe domain may also mediate the interaction between effector molecules and cells, which may lead to activation of the immune system. In the IgG, IgA, and IgD antibody isotypes, the Fc region may comprise two identical protein fragments, which can be derived from the second and third constant domains of the antibody's heavy chains. In the IgM and IgE antibody isotypes, the Fc regions may comprise three heavy chain constant domains. In the IgG antibody isotype, the Fc regions may comprise a highly-conserved N-glycosylation site, which may be important for FcR-mediated downstream effects.

An antibody used herein may be chimeric or “humanized.” Chimeric and humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which may contain minimal sequences derived from non-human immunoglobulin. In general, the humanized antibody may comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may also comprise at least a portion of an immunoglobulin constant region (Fc), typically that of a human immunoglobulin consensus sequence.

An antibody described herein may be a human antibody. As used herein, “human antibodies” can include antibodies having, for example, the amino acid sequence of a human immunoglobulin and may include antibodies isolated from human immunoglobulin libraries or from animals transgenic for one or more human immunoglobulins that do not express endogenous immunoglobulins. Human antibodies may be produced using transgenic mice which are incapable of expressing functional endogenous immunoglobulins, but which may express human immunoglobulin genes. Completely human antibodies that recognize a selected epitope may be generated using guided selection. In this approach, a selected non-human monoclonal antibody, e.g., a mouse antibody, may be used to guide the selection of a completely human antibody recognizing the same epitope.

An antibody described herein may be a bispecific antibody or a dual variable domain antibody (DVD). Bispecific and DVD antibodies may be monoclonal, often human or humanized, antibodies that can have binding specificities for at least two different antigens.

An antibody described herein may be a derivatized antibody. For example, derivatized antibodies may be modified by glycosylation, acetylation, pegylation, phosphorylation, amidation, derivatization by known protecting/blocking groups, proteolytic cleavage, linkage to a cellular ligand or other protein.

An antibody described herein may have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding may be reduced by, for example, mutating the immunoglobulin constant region segment of the antibody at particular regions necessary for FcR interactions.

An antibody or Fc domain as described herein may be modified to acquire or improve at least one constant region-mediated biological effector function relative to an unmodified antibody or Fc domain, e.g., to enhance FcγR interactions. For example, an antibody with a constant region that binds FcγRIIA, FcγRIIB and/or FcγRIIIA with greater affinity than the corresponding wild type constant region may be produced according to the methods described herein. An Fc domain that binds FcγRIIA, FcγRIIB and/or FcγRIIIA with greater affinity than the corresponding wild type Fc domain may be produced according to the methods described herein.

In certain embodiments, the antibody construct comprises an antigen binding domain and an IgG Fc domain, wherein a K_{d} for binding of the antigen binding domain to a first antigen in a presence of the immune-stimulatory compound is less than about 100 nM and no greater than about 100 times a K_{d} for binding of the antigen binding domain to the first antigen in the absence of the immune-stimulatory compound. In certain embodiments, the antibody construct comprises a K_{d} for binding of the IgG Fc domain to an Fc receptor in the presence of the immune-stimulatory compound is no greater than about 100 times a K_{d} for binding the IgG Fc domain to the Fc receptor in the absence of the immune-stimulatory compound. In certain embodiments, the first antigen is selected from CD5, CD19, CD20, CD25, CD37, CD30, CD33, CD40, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), GD2, GD3, GM2, Le^{y}, CA-125, CA19-9, epidermal growth factor, p185HER2, IL-2 receptor, EGFRvIII (de2-7 EGFR), fibroblast activation protein, tenascin, a metalloproteinase, endosialin, vascular endothelial growth factor, avB3, WT1, LMP2, HPV E6, HPV E7, Her-2/neu, MAGE A3, p53 nonmutant, NY-ESO-1, MelanA/MART1, Ras mutant, gp100, p53 mutant, PR1, bcr-abl, tyronsinase, survivin, PSA, hTERT, a Sarcoma translocation breakpoint protein, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TESL Sperm protein 17, LCK, HMWMAA, AKAP-4, SSX2, XAGE 1, Legumain, Tie 3, VEGFR2, MAD-CT-1, PDGFR-B, MAD-CT-2, ROR2, TRAIL1, MUC16, MAGE A4, MAGE C2, GAGE, EGFR, CMET, HER3, MUC15, CA6, NAPI2B, TROP2, CLDN6, CLDN16, CLDN18.2, CLorfl86, RON, LY6E, FRA, DLL3, PTK7, STRA6, TMPRSS3, TMPRSS4, TMEM238, UPK1B, VTCN1, LIV1, ROR1, or Fos-related antigen 1. In certain embodiments, an antigen binding domain specifically binds to an antigen, such as those selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In certain embodiments, the first antigen is expressed on an immune cell. In certain embodiments, the first antigen is CD40, HER2 or TROP2. In certain embodiments, the first antigen is HER2 or TROP2.

In certain embodiments, the antibody construct comprises a human antibody or a humanized antibody or an antigen binding portion thereof, e.g., a humanized CD40, humanized HER2 or humanized TROP2 antibody. In certain embodiments, the antibody construct comprises a TROP2 antibody, e.g., sacituzumab, or an antigen binding portion thereof. In certain embodiments, the antibody construct comprises the heavy and light chain variable region sequences of sacituxumab (SEQ ID NOs:3 and 4). In certain embodiments, the antibody construct comprises LC CDR1, LC CDR2 and LC CDR3 of the light chain variable region of sacituzumab (SEQ ID NO:4), and HC CDR1, HC CDR2 and HC CDR3 of the heavy chain variable region of sacituzumab (SEQ ID NO:3), as determined by the Kabat index. In certain embodiments, the antibody construct comprises LC CDR1, LC CDR2 and LC CDR3 of the light chain variable region of sacituzumab (SEQ ID NO:4), and HC CDR1, HC CDR2 and HC CDR3 of the heavy chain variable region of sacituzumab (SEQ ID NO:3), as determined by IMGT (ImMunoGeneTics). In certain embodiments, the antibody construct comprises a HER2 antibody, e.g., pertuzumab, trastuzumab, or an antigen binding portion thereof. In certain embodiments, the antibody construct comprises the heavy and light chain variable region sequences of pertuzumab (SEQ ID NOs:1 and 2). In certain embodiments, the antibody construct comprises LC CDR1, LC CDR2 and LC CDR3 of the light chain variable region of pertuzumab (SEQ ID NO:2), and HC CDR1, HC CDR2 and HC CDR3 of the heavy chain variable region of pertuzumab (SEQ ID NO:1), as determined by the Kabat index. In certain embodiments, the antibody construct comprises LC CDR1, LC CDR2 and LC CDR3 of the light chain variable region of pertuzumab (SEQ ID NO:2), and HC CDR1, HC CDR2 and HC CDR3 of the heavy chain variable region of pertuzumab (SEQ ID NO:1), as determined by IMGT. In certain embodiments, the antibody construct comprises the heavy and light chain variable region sequences of trastuzumab (SEQ ID NOs:7 and 8). In certain embodiments, the antibody construct comprises LC CDR1, LC CDR2 and LC CDR3 of the light chain variable region of trastuzumab (SEQ ID NO:8), and HC CDR1, HC CDR2 and HC CDR3 of the heavy chain variable region of trastuzumab (SEQ ID NO:7), as determined by the Kabat index. In certain embodiments, the antibody construct comprises LC CDR1, LC CDR2 and LC CDR3 of the light chain variable region of trastuzumab (SEQ ID NO:8), and HC CDR1, HC CDR2 and HC CDR3 of the heavy chain variable region of trastuzumab (SEQ ID NO:7), as determined by IMGT. In certain embodiments, the antibody construct comprises a CD40 antibody or an antigen binding portion thereof. In certain embodiments, the antibody construct comprises the heavy and light chain variable region sequence of sacituxumab (SEQ ID NO:3 and 4). In certain embodiments, the antibody construct comprises the LC CDR1, LC CDR2 and LC CDR3 of the light chain variable region of sacituzumab (SEQ ID NO:4), and HC CDR1, HC CDR2 and HC CDR3 of the heavy chain variable region of sacituzumab (SEQ ID NO:3), as determined by Kabat index.

In certain embodiments, the antibody construct comprises a Liv-1 antibody, e.g., ladiratuzumab, huLiv1-14 (WO 2012078688), Liv1-1.7A4 (US 2011/0117013), huLiv1-22 (WO 2012078688) or an antigen binding portion thereof. In certain embodiments, the antibody construct comprises the heavy and light chain variable region sequences of ladiratuzumab (SEQ ID NOs:5 and 6). In certain embodiments, the antibody construct comprises LC CDR1, LC CDR2 and LC CDR3 of the light chain variable region of ladiratuzumab (SEQ ID NO:6), and HC CDR1, HC CDR2 and HC CDR3 of the heavy chain variable region of ladiratuzumab (SEQ ID NO:5), as determined by Kabat index. In certain embodiments, the antibody construct comprises LC CDR1, LC CDR2 and LC CDR3 of the light chain variable region of ladiratuzumab (SEQ ID NO:6), and HC CDR1, HC CDR2 and HC CDR3 of the heavy chain variable region of ladiratuzumab (SEQ ID NO:5), as determined by IMGT. In certain embodiments, the antibody construct comprises the heavy and light chain variable region sequences of huLiv1-14 (SEQ ID NOs:17 and 18). In certain embodiments, the antibody construct comprises LC CDR1, LC CDR2 and LC CDR3 of the light chain variable region of huLiv1-14 (SEQ ID NO:18), and HC CDR1, HC CDR2 and HC CDR3 of the heavy chain variable region of huLiv1-14 (SEQ ID NO:17), as determined by Kabat index. In certain embodiments, the antibody construct comprises LC CDR1, LC CDR2 and LC CDR3 of the light chain variable region of huLiv1-14 (SEQ ID NO:18), and HC CDR1, HC CDR2 and HC CDR3 of the heavy chain variable region of huLiv1-14 (SEQ ID NO:17), as determined by IMGT. In certain embodiments, the antibody construct comprises the heavy and light chain variable region sequences of Liv1-1.7A4 (SEQ ID NOs:19 and 20). In certain embodiments, the antibody construct comprises LC CDR1, LC CDR2 and LC CDR3 of the light chain variable region of Liv1-1.7A4 (SEQ ID NO:20), and HC CDR1, HC CDR2 and HC CDR3 of the heavy chain variable region of Liv1-1.7A4 (SEQ ID NO:19), as determined by Kabat index. In certain embodiments, the antibody construct comprises a humanized antibody or antigen binding fragment thereof comprising LC CDR1, LC CDR2 and LC CDR3 of the light chain variable region of Liv1-1.7A4 (SEQ ID NO:20), and HC CDR1, HC CDR2 and HC CDR3 of the heavy chain variable region of Liv1-1.7A4 (SEQ ID NO:19), as determined by Kabat index. In certain embodiments, the antibody construct comprises LC CDR1, LC CDR2 and LC CDR3 of the light chain variable region of Liv1-1.7A4 (SEQ ID NO:20), and HC CDR1, HC CDR2 and HC CDR3 of the heavy chain variable region of Liv1-1.7A4 (SEQ ID NO:19), as determined by IMGT. In certain embodiments, the antibody construct comprises a humanized antibody or antigen binding fragment thereof comprising LC CDR1, LC CDR2 and LC CDR3 of the light chain variable region of Liv1-1.7A4 (SEQ ID NO:20), and HC CDR1, HC CDR2 and HC CDR3 of the heavy chain variable region of Liv1-1.7A4 (SEQ ID NO:19), as determined by IMGT. In certain embodiments, the antibody construct comprises the heavy and light chain variable region sequences of huLiv1-22 (SEQ ID NOs:21 and 22). In certain embodiments, the antibody construct comprises LC CDR1, LC CDR2 and LC CDR3 of the light chain variable region of huLiv1-22 (SEQ ID NO:22), and HC CDR1, HC CDR2 and HC CDR3 of the heavy chain variable region of huLiv1-22 (SEQ ID NO:21), as determined by Kabat index. In certain embodiments, the antibody construct comprises LC CDR1, LC CDR2 and LC CDR3 of the light chain variable region of huLiv1-22 (SEQ ID NO:22), and HC CDR1, HC CDR2 and HC CDR3 of the heavy chain variable region of huLiv1-22 (SEQ ID NO:21), as determined by IMGT.

In certain embodiments, the antibody construct comprises a MUC16 antibody, e.g., sofituzumab, 4H11 (US2013/0171152), 4H5 (US2013/0171152) or an antigen binding portion thereof. In certain embodiments, the antibody construct comprises the heavy and light chain variable region sequences of sofituzumab (SEQ ID NOs:23 and 24). In certain embodiments, the antibody construct comprises LC CDR1, LC CDR2 and LC CDR3 of the light chain variable region of sofituzumab (SEQ ID NO:24), and HC CDR1, HC CDR2 and HC CDR3 of the heavy chain variable region of sofituzumab (SEQ ID NO:23), as determined by Kabat index. In certain embodiments, the antibody construct comprises LC CDR1, LC CDR2 and LC CDR3 of the light chain variable region of sofituzumab (SEQ ID NO:24), and HC CDR1, HC CDR2 and HC CDR3 of the heavy chain variable region of sofituzumab (SEQ ID NO:23), as determined by IMGT. In certain embodiments, the antibody construct comprises the heavy and light chain variable region sequences of antibody 4H11 (SEQ ID NOs:13 and 14). In certain embodiments, the antibody construct comprises LC CDR1, LC CDR2 and LC CDR3 of the light chain variable region of antibody 4H11 (SEQ ID NO:14), and HC CDR1, HC CDR2 and HC CDR3 of the heavy chain variable region of antibody 4H11 (SEQ ID NO:13), as determined by Kabat index. In certain embodiments, the antibody construct comprises a humanized antibody comprising LC CDR1, LC CDR2 and LC CDR3 of the light chain variable region of antibody 4H11 (SEQ ID NO:14), and HC CDR1, HC CDR2 and HC CDR3 of the heavy chain variable region of antibody 4H11 (SEQ ID NO:13), as determined by Kabat index. In certain embodiments, the antibody construct comprises LC CDR1, LC CDR2 and LC CDR3 of the light chain variable region of antibody 4H11 (SEQ ID NO:14), and HC CDR1, HC CDR2 and HC CDR3 of the heavy chain variable region of 4H11 (SEQ ID NO:13), as determined by IMGT. In certain embodiments, the antibody construct comprises a humanized antibody or antigen binding fragment thereof comprising LC CDR1, LC CDR2 and LC CDR3 of the light chain variable region of antibody 4H11 (SEQ ID NO:14), and HC CDR1, HC CDR2 and HC CDR3 of the heavy chain variable region of 4H11 (SEQ ID NO:13), as determined by IMGT. In certain embodiments, the antibody construct comprises the heavy and light chain variable region sequences of antibody 4A5 (SEQ ID NOs:15 and 16). In certain embodiments, the antibody construct comprises LC CDR1, LC CDR2 and LC CDR3 of the light chain variable region of antibody 4A5 (SEQ ID NO:16), and HC CDR1, HC CDR2 and HC CDR3 of the heavy chain variable region of 4A5 (SEQ ID NO:15), as determined by Kabat index. In certain embodiments, the antibody construct comprises a humanized antibody or an antigen binding fragment thereof comprising LC CDR1, LC CDR2 and LC CDR3 of the light chain variable region of antibody 4A5 (SEQ ID NO:16), and HC CDR1, HC CDR2 and HC CDR3 of the heavy chain variable region of antibody 4A5 (SEQ ID NO:15), as determined by Kabat index. In certain embodiments, the antibody construct comprises LC CDR1, LC CDR2 and LC CDR3 of the light chain variable region of antibody 4A5 (SEQ ID NO:16), and HC CDR1, HC CDR2 and HC CDR3 of the heavy chain variable region of antibody 4A5 (SEQ ID NO:15), as determined by IMGT. In certain embodiments, the antibody construct comprises a humanized antibody or antigen binding fragment thereof comprising LC CDR1, LC CDR2 and LC CDR3 of the light chain variable region of 4A5 (SEQ ID NO:16), and HC CDR1, HC CDR2 and HC CDR3 of the heavy chain variable region of 4A5 (SEQ ID NO:15), as determined by IMGT.

In certain embodiments, the antibody construct comprises a PD-L1 antibody, e.g., atezolizumab, MDX-1105 (WO 2007/005874) or an antigen binding portion thereof. In certain embodiments, the antibody construct comprises the heavy and light chain variable region sequences of atezolizumab (SEQ ID NOs:11 and 12). In certain embodiments, the antibody construct comprises LC CDR1, LC CDR2 and LC CDR3 of the light chain variable region of atezolizumab (SEQ ID NO:12), and HC CDR1, HC CDR2 and HC CDR3 of the heavy chain variable region of atezolizumab (SEQ ID NO:11), as determined by Kabat index. In certain embodiments, the antibody construct comprises LC CDR1, LC CDR2 and LC CDR3 of the light chain variable region of atezolizumab (SEQ ID NO:12), and HC CDR1, HC CDR2 and HC CDR3 of the heavy chain variable region of atezolizumab (SEQ ID NO:11), as determined by IMGT. In certain embodiments, the antibody construct comprises the heavy and light chain variable region sequences of MDX-1105 (SEQ ID NOs:9 and 10). In certain embodiments, the antibody construct comprises LC CDR1, LC CDR2 and LC CDR3 of the light chain variable region of MDX-1105 (SEQ ID NO:10), and HC CDR1, HC CDR2 and HC CDR3 of the heavy chain variable region of MDX-1105 (SEQ ID NO:9), as determined by Kabat index. In certain embodiments, the antibody construct comprises a humanized antibody or antigen binding fragment thereof comprising LC CDR1, LC CDR2 and LC CDR3 of the light chain variable region of MDX-1105 (SEQ ID NO:10), and HC CDR1, HC CDR2 and HC CDR3 of the heavy chain variable region of MDX-1105 (SEQ ID NO:9), as determined by Kabat index. In certain embodiments, the antibody construct comprises LC CDR1, LC CDR2 and LC CDR3 of the light chain variable region of MDX-1105 (SEQ ID NO:10), and HC CDR1, HC CDR2 and HC CDR3 of the heavy chain variable region of MDX-1105 (SEQ ID NO:9), as determined by IMGT. In certain embodiments, the antibody construct comprises a humanized antibody or antigen binding fragment thereof comprising LC CDR1, LC CDR2 and LC CDR3 of the light chain variable region of MDX-1105 (SEQ ID NO:10), and HC CDR1, HC CDR2 and HC CDR3 of the heavy chain variable region of MDX-1105 (SEQ ID NO:9), as determined by IMGT.

An antibody construct may comprise an antibody with modifications of at least one amino acid residue. Modifications may be substitutions, additions, mutations, deletions, or the like. An antibody modification can be an insertion of an unnatural amino acid.

The exemplary antibody construct V_{H} sequences and V_{L} sequences are illustrated in Table A below.

TABLE A
Exemplary Antibody Construct VH sequences and VL sequences

| | | SEQ ID | |
|---|---|---|---|
| Antibody | Region | NO: | Sequence |
| Pertuzumab | V_{H} | 1 | EVQLVESGGGLVQPGGSLRLSCAASGFTFTDYT |
| | | | MDWVRQAPGKGLEWVADVNPNSGGSIYNQRF |
| | | | KGRFTLSVDRSKNTLYLQMNSLRAEDTAVYYC |
| | | | ARNLGPSFYFDYWGQGTLVTVSS |
| | V_{L} | 2 | DIQMTQSPSSLSASVGDRVTITCKASQDVSIGV |
| | | | AWYQQKPGKAPKLLIYSASYRYTGVPSRFSGS |
| | | | GSGTDFTLTISSLQPEDFATYYCQQYYIYPYTFG |
| | | | QGTKVEIK |
| Sacituzumab | V_{H} | 3 | QVQLQQSGSELKKPGASVKVSCKASGYTFTNY |
| | | | GMNWVKQAPGQGLKWMGWINTYTGEPTYTD |
| | | | DFKGRFAFSLDTSVSTAYLQISSLKADDTAVYF |
| | | | CARGGFGSSYWYFDVWGQGSLVTVSS |
| | V_{L} | 4 | DIQLTQSPSSLSASVGDRVSITCKASQDVSIAVA |
| | | | WYQQKPGKAPKLLIYSASYRYTGVPDRFSGSG |
| | | | SGTDFTLTISSLQPEDFAVYYCQQHYITPLTFGA |
| | | | GTKVEIK |
| Ladiratuzumab | V_{H} | 5 | QVQLVQSGAEVKKPGASVKVSCKASGLTIEDY |
| | | | YMHWVRQAPGQGLEWMGWIDPENGDTEYGP |
| | | | KFQGRVTMTRDTSINTAYMELSRLRSDDTAVY |
| | | | YCAVHNAHYGTWFAYWGQGTLVTVSS |
| | V_{L} | 6 | DVVMTQSPLSLPVTLGQPASISCRSSQSLLHSSG |
| | | | NTYLEYFQQRPGQSPRPLIYKISTRFSGVPDRFS |
| | | | GSGSGTDFTLKISRVEAEDVGVYYCFQGSHVP |
| | | | YTFGGGTKVEIK |
| Trastuzumab | V_{H} | 7 | EVQLVESGGGLVQPGGSLRLSCAASGFNIKDTY |
| | | | IHWVRQAPGKGLEWVARIYPTNGYTRYADSV |
| | | | KGRFTISADTSKNTAYLQMNSLRAEDTAVYYC |
| | | | SRWGGDGFYAMDYWGQGTLVTVSS |
| | V_{L} | 8 | DIQMTQSPSSLSASVGDRVTITCRASQDVNTAV |
| | | | AWYQQKPGKAPKLLIYSASFLYSGVPSRFSGSR |
| | | | SGTDFTLTISSLQPEDFATYYCQQHYTTPPTFGQ |
| | | | GTKVEIK |
| MDX-1105 | V_{H} | 9 | QVQLVQSGAEVKKPGSSVKVSCKTSGDTFSTY |
| | | | AISWVRQAPGQGLEWMGGIIPIFGKAHYAQKF |
| | | | QGRVTITADESTSTAYMELSSLRSEDTAVYFCA |
| | | | RKFHFVSGSPFGMDVWGQGTTVTVSS |
| | V_{L} | 10 | EIVLTQSPATLSLSPGERATLSCRASQSVSSYLA |
| | | | WYQQKPGQAPRLLIYDASNRATGIPARFSGSGS |
| | | | GTDFTLTISSLEPEDFAVYYCQQRSNWPTFGQG |
| | | | TKVEIK |
| Atezolizumab | V_{H} | 11 | EVQLVESGGGLVQPGGSLRLSCAASGFTFSDS |
| | | | WIHWVRQAPGKGLEWVAWISPYGGSTYYADS |
| | | | VKGRFTISADTSKNTAYLQMNSLRAEDTAVYY |
| | | | CARRHWPGGFDYWGQGTLVTVSS |
| | V_{L} | 12 | DIQMTQSPSSLSASVGDRVTITCRASQDVSTAV |
| | | | AWYQQKPGKAPKLLIYSASFLYSGVPSRFSGSG |
| | | | SGTDFTLTISSLQPEDFATYYCQQYLYHPATFG |
| | | | QGTKVEIK |
| 4H11 | V_{H} | 13 | EVKLQESGGGFVKPGGSLKVSCAASGFTFSSYA |
| | | | MSWVRLSPEMRLEWVATISSAGGYIFYSDSVQ |
| | | | GRFTISRDNAKNTLHLQMGSLRSGDTAMYYCA |
| | | | RQGFGNYGDYYAMDYWGQGTTVTVSS |
| | V_{L} | 14 | DIELTQSPSSLAVSAGEKVTMSCKSSQSLLNSRT |
| | | | RKNQLAWYQQKPGQSPELLIYWASTRQSGVPD |
| | | | RFTGSGSGTDFTLTISSVQAEDLAVYYCQQSYN |
| | | | LLTFGPGTKLEVK |
| 4A5 | V_{H} | 15 | EVKLEESGGGFVKPGGSLKISCAASGFTFRNYA |
| | | | MSWVRLSPEMRLEWVATISSAGGYIFYSDSVQ |
| | | | GRFTISRDNAKNTLHLQMGSLRSGDTAMYYCA |
| | | | RQGFGNYGDYYAMDYWGQGTTVTVSS |
| | V_{L} | 16 | DIELTQSPSSLAVSAGEKVTMSCKSSQSLLNSRT |
| | | | RKNQLAWYQQKTGQSPELLIYWASTRQSGVPD |
| | | | RFTGSGSGTDFTLTISSVQAEDLAVYYCQQSYN |
| | | | LLTFGPGTKLEIK |
| huLiv1-14 | V_{H} | 17 | QVQLVQSGAEVKKPGASVKVSCKASGYTIEDY |
| | | | YMHWVRQAPGQGLEWMGWIDPENGDTEYAP |
| | | | TFQGRVTMTRDTSINTAYMELSRLRSDDTAVY |
| | | | YCARHDAHYGTWFAYWGQGTLVTVSS |
| | V_{L} | 18 | DVVMTQSPLSLPVTLGQPASISCRSSQSIIRNDG |
| | | | NTYLEWYQQRPGQSPRRLIYRVSNRFSGVPDRF |
| | | | SGSGSGTDFTLKISRVEAEDVGVYYCFQGSHVP |
| | | | YTFGGGTKVEIK |
| Liv1-1.7A4 | V_{H} | 19 | EIQLQQSGPELMKPGASVKISCKASTYSFTRYF |
| | | | MHWVKQSHGESLEWIGYIDPFNGGTGYNQKF |
| | | | KGKATLTVDKSSSTAYMHLSSLTSEDSAVYYC |
| | | | VTYGSDYFDYWGQGTTLTVSS |
| | V_{L} | 20 | DIVMTQPQKFMSTSVGDRVSVTCKASQNVETD |
| | | | VVWYQQKPGQPPKALIYSASYRHSGVPDRFTG |
| | | | SGSGTNFTLTISTVQSEDLAEYFCQQYNNYPFT |
| | | | FGSGTKLEIIR |
| huLiv1-22 | V_{H} | 21 | QVQLVQSGAEVKKPGASVKVSCKASGLTIEDY |
| | | | YMHWVRQAPGQGLEWMGWIDPENGDTEYGP |
| | | | KFQGRVTMTRDTSINTAYMELSRLRSDDTAVY |
| | | | YCAVHNAHYGTWFAYWGQGTLVTVSS |
| | V_{L} | 22 | DVVMTQSPLSLPVTLGQPASISCRSSQSLLHSSG |
| | | | NTYLEWYQQRPGQSPRPLIYKISTRFSGVPDRFS |
| | | | GSGSGTDFTLKISRVEAEDVGVYYCFQGSHVP |
| | | | YTFGGGTKVEIK |
| Sofituzumab | VH | 23 | EVQLVESGGGLVQPGGSLRLSCAASGYSITNDY |
| | | | AWNWVRQAPGKGLEWVGYISYSGYTTYNPSL |
| | | | KSRFTISRDTSKNTLYLQMNSLRAEDTAVYYC |
| | | | ARWTSGLDYWGQGTLVTVSS |
| | VL | 24 | DIQMTQSPSSLSASVGDRVTITCKASDLIHNWL |
| | | | AWYQQKPGKAPKLLIYGATSLETGVPSRFSGS |
| | | | GSGTDFTLTISSLQPEDFATYYCQQYWTTPFTF |
| | | | GQGTKVEIK |

### Target Binding Domain of an Antibody Construct

An antibody construct may further comprise a target binding domain. A target binding domain may comprise a domain that binds to a target. A target may be an antigen. A target binding domain may comprise an antigen binding domain. A target binding domain may be a domain that can specifically bind to an antigen. A target binding domain may be an antigen-binding portion of an antibody or an antibody fragment. A target binding domain may be one or more fragments of an antibody that can retain the ability to specifically bind to an antigen. A target binding domain may be any antigen binding fragment. A target binding domain may be in a scaffold, in which a scaffold is a supporting framework for the antigen binding domain. A target binding domain may comprise an antigen binding domain in a scaffold.

A target binding domain may comprise an antigen binding domain which can refer to a portion of an antibody comprising the antigen recognition portion, i.e., an antigenic determining variable region of an antibody sufficient to confer recognition and binding of the antigen recognition portion to a target, such as an antigen, i.e., the epitope. A target binding domain may comprise an antigen binding domain of an antibody. In certain embodiments, a target binding domain is a CD40 agonist.

An Fv can be the minimum antibody fragment which contains a complete antigen-recognition and antigen-binding site. This region may consist of a dimer of one heavy chain and one light chain variable domain in tight, non-covalent association. In this configuration, the three hypervariable regions of each variable domain may interact to define an antigen-binding site on the surface of the V_{H}-V_{L} dimer. A single variable domain (or half of an Fv comprising only three hypervariable regions specific for an antigen) can recognize and bind antigen, although at a lower affinity than the entire binding site.

A target binding domain may be at least 80% homologous to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a single chain variable fragment (scFv), or from a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor. In certain embodiments, a target binding domain may be at least 80% homologous to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a single chain variable fragment (scFv)

A target binding domain may be attached to an antibody construct. For example, an antibody construct may be fused with a target binding domain to create an antibody construct target binding domain fusion. The antibody construct-target binding domain fusion may be the result of the nucleic acid sequence of the target binding domain being expressed in frame with the nucleic acid sequence of the antibody construct. The antibody construct-target binding domain fusion may be the result of an in-frame genetic nucleotide sequence encoding the antibody construct and the target binding domain or may be a contiguous protein sequence. As another example, a target binding domain may be linked to an antibody construct. A target binding domain may be linked to an antibody construct by a chemical conjugation. A target binding domain may be attached to a terminus of an Fc region. A target binding domain may be attached to a terminus of an Fc region. A target binding domain may be attached to a terminus of an antibody construct. A target binding domain may be attached to a terminus of an antibody. A target binding domain may be attached to a light chain of an antibody. A target binding domain may be attached to a terminus of a light chain of an antibody. A target binding domain may be attached to a heavy chain of an antibody. A target binding domain may be attached to terminus of a heavy chain of an antibody. The terminus may be a C-terminus. An antibody construct may be attached to 1, 2, 3, and/or 4 target binding domains. The target binding domain may direct the antibody construct to, for example, a particular cell or cell type. A target binding domain of an antibody construct may be selected in order to recognize an antigen, e.g., an antigen expressed on an immune cell. An antigen can be a peptide or fragment thereof. An antigen may be expressed on an antigen-presenting cell. An antigen may be expressed on a dendritic cell, a macrophage, or a B cell. As another example, an antigen may be a tumor antigen. The tumor antigen may be any tumor antigen described herein. When multiple target binding domains are attached to an antibody construct, the target binding domains may bind to the same antigen. When multiple target binding domains are attached to an antibody construct, the target binding domains may bind different antigens.

In certain embodiments, an antibody construct described herein specifically binds a second antigen. In certain embodiments, the target binding domain is linked, e.g., covalently bound, to the antibody construct at a C-terminal end of the Fc domain.

### Compounds

The following is a discussion of compounds and salts thereof that may be used in the methods of the disclosure. The compounds and salts described in Formulas (IA), (IB), (IC), (VIIA), (VIIB), and (VIIC), may be covalently bound, to linkers, L³, which may further be covalently bound to antibody constructs. The compound and salts described in Formulas (IIA), (IIB), (IIC), (VIIIA), (VIIIB), and (VIIIC) are the compounds of Formulas (IA), (IB), (IC), (VIIA), (VIIB), and (VIIC) covalently bound to linkers, L³, which may further be covalently bound to antibody constructs.

In some aspects, the present disclosure provides a compound represented by Formula (IA):

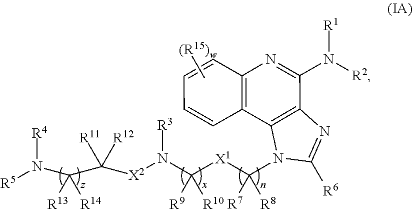

or a salt thereof, wherein:

R¹, R², R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In certain embodiments, the present disclosure provides a compound or salt of Formula (IA), wherein:

R¹, R², R³, R⁴, and R⁵ are independently selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂;

n is 1 or 2;

x is 1 or 2;

w is 0, 1, or 2; and

z is 0, 1, or 2.

In certain embodiments, the present disclosure provides a compound or salt of Formula (IA), wherein:

R¹, R², R³, R⁴, and R⁵ are independently selected from hydrogen; and C₁₋₆ alkyl, optionally substituted with one or more substituents independently selected from halogen, —OR²⁰, —SR²⁰, —N(R²⁰)₂, and —CN;

R⁶ is selected from halogen and C₁₋₆ alkyl, optionally substituted with one or more substituents independently selected from halogen, —OR²⁰, —SR²⁰, and —CN;

R⁷, R⁸, R⁹, and R¹⁰ are independently selected at each occurrence from hydrogen and halogen; and C₁₋₆ alkyl, optionally substituted with one or more substituents independently selected from halogen;

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹³ and R¹⁴ are independently selected at each occurrence from hydrogen, halogen, —OR²⁰, —SR²⁰, and —CN; C₁₋₆ alkyl, optionally substituted with one or more substituents independently selected from halogen, —OR²⁰, —SR²⁰, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂;

n is 1 or 2;

x is 1 or 2;

w is 0, 1, or 2; and

z is 0, 1, or 2.

In certain embodiments, X¹ is O. In certain embodiments, n is 2. In certain embodiments, x is 2. In certain embodiments, z is 0. In certain embodiments, z is 1

In some aspects, the present disclosure provides a compound represented by Formula (IB).

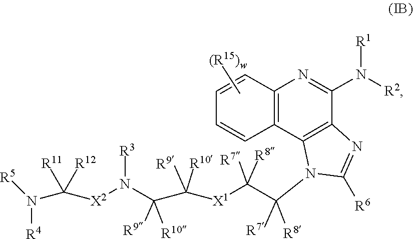

or a salt thereof, wherein.

R¹, R², R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, 2, 3, or 4.

In certain embodiments, the present disclosure provides a compound or salt of Formula (IB), wherein:

R¹, R², R³, R⁴, and R⁵ are independently selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, or 2.

In certain embodiments, the present disclosure provides a compound or salt of Formula (IB), wherein:

R¹, R², R³, R⁴, and R⁵ are independently selected from hydrogen; and C₁₋₆ alkyl, optionally substituted with one or more substituents independently selected from halogen, —OR²⁰, —SR²⁰, —N(R²⁰)₂, and —CN;

R⁶ is selected from halogen and C₁₋₆ alkyl, optionally substituted with one or more substituents independently selected from halogen, —OR²⁰, —SR²⁰, and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen;

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, or 2.

In some aspects, the present disclosure provides a compound represented by Formula (IC):

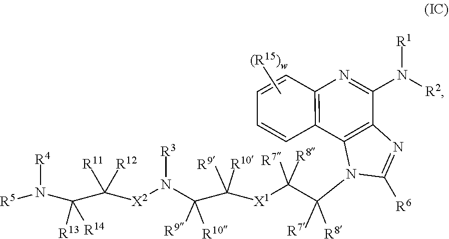

or a salt thereof, wherein:

R¹, R², R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, and —CN; C₁₋₆ alkyl, optionally substituted with one or more substituents independently selected from halogen, —OR²⁰, —SR²⁰, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, 2, 3, or 4.

In certain embodiments, the present disclosure provides a compound or salt of Formula (IC), wherein:

R¹, R², R³, R⁴, and R⁵ are independently selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹³ and R¹⁴ are independently selected at each occurrence from hydrogen, halogen, —OR²⁰, —SR²⁰, and —CN; C₁₋₆ alkyl, optionally substituted with one or more substituents independently selected from halogen, —OR²⁰, —SR²⁰, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, or 2.

In certain embodiments, the present disclosure provides a compound or salt of Formula (IC), wherein:

R¹, R², R³, R⁴, and R⁵ are independently selected from hydrogen; and C₁₋₆ alkyl, optionally substituted with one or more substituents independently selected from halogen, —OR²⁰, —SR²⁰, —N(R²⁰)₂, and —CN;

R⁶ is selected from halogen and C₁₋₆ alkyl, optionally substituted with one or more substituents independently selected from halogen, —OR²⁰, —SR²⁰, and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen;

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹³ and R¹⁴ are independently selected at each occurrence from hydrogen, halogen, —OR²⁰, —SR²⁰, and —CN; C₁₋₆ alkyl, optionally substituted with one or more substituents independently selected from halogen, —OR²⁰, —SR²⁰, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, or 2.

In certain embodiments, for a compound or salt of Formula (IA), (IB), or (IC), R¹, R², R³, R⁴, and R⁵ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN.

In certain embodiments, R¹ and R² are independently selected from hydrogen and C₁₋₆ alkyl. In certain embodiments, R¹ is hydrogen or C₁₋₃ alkyl. In certain embodiments, R² is hydrogen or C₁₋₃ alkyl. In certain embodiments, R¹ and R² are both hydrogen. In certain embodiments, R³ is selected from hydrogen and C₁₋₆ alkyl optionally substituted with one or more halogens. In certain embodiments, R³ is hydrogen. In certain embodiments, R⁴ is selected from hydrogen and C₁₋₆ alkyl optionally substituted with one or more halogens. In certain embodiments, R⁴ is hydrogen. In certain embodiments, R⁵ is selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In certain embodiments, R⁵ is hydrogen.

In certain embodiments, R¹, R², R³, R⁴, and R⁵ are independently selected from hydrogen and C₁₋₆ alkyl optionally substituted with one or more halogens. In certain embodiments, R¹, R², R³, R⁴, and R⁵ are independently selected from hydrogen and C₁₋₆ alkyl. In certain embodiments, R¹, R², and R³ are hydrogen. In certain embodiments, for a compound or salt of Formula (IA), (IB), or (IC), R¹, R², R⁴, and R⁵ are hydrogen. In certain embodiments, for a compound or salt of Formula (IA), (IB), or (IC), R¹, R², R³, R⁴, and R⁵ are hydrogen.

In certain embodiments, R⁶ is selected from halogen, —OR²⁰, and —N(R²⁰)₂; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle. In certain embodiments, R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle. In certain embodiments, R⁶ is C₁₋₆ alkyl substituted with —OR²⁰, and R²⁰ is selected from hydrogen and C₁₋₆ alkyl optionally substituted with one or more substituents independently selected from halogen, —OH, and —NH₂. In certain embodiments, R⁶ is C₁₋₃ alkyl substituted with —OR²⁰, and R²⁰ is selected from hydrogen and C₁₋₃ alkyl. In certain embodiments, R⁶ is C₁ alkyl substituted with —OR²⁰, and R²⁰ is C₁₋₃ alkyl. In certain embodiments, R⁶ is C₂ alkyl substituted with —OR²⁰, and R²⁰ is C₂₋₃ alkyl.

In certain embodiments, for a compound or salt of Formula (IA), (IB), or (IC), R¹, R², R³, R⁴, and R⁵ are hydrogen, and R⁶ is C₁₋₃ alkyl substituted with —OR²⁰, and R²⁰ is selected from hydrogen and C₁₋₃ alkyl. In certain embodiments, for a compound or salt of Formula (IA), (IB), or (IC), R¹, R², R³, R⁴, and R⁵ are hydrogen, and R⁶ is C₁ alkyl substituted with —OR²⁰, and R²⁰ is selected from hydrogen and C₁₋₃ alkyl.

In certain embodiments, R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen. In certain embodiments, R^{7′} and R^{8′} are each hydrogen. In certain embodiments, R^{7″} and R^{8″} are each C₁₋₆ alkyl. In certain embodiments, R^{7″} and R^{8″} are each methyl. In certain embodiments, R^{9′}, R^{9″}, R^{10′}, and R^{10″} are independently selected at each occurrence from hydrogen and C₁₋₆ alkyl. In certain embodiments, R^{9′}, R^{9″}, R^{10′}, and R^{10″} are each hydrogen

In certain embodiments, R^{7′} and R^{8′} are each hydrogen, R^{7″} and R^{8″} are each C₁₋₆ alkyl, and R^{9′}, R^{9″}, R^{10′}, and R^{10″} are independently selected at each occurrence from hydrogen and C₁₋₆ alkyl. In certain embodiments, R^{7′} and R^{8′} are each hydrogen, R^{7″} and R^{8″} are each C₁₋₆ alkyl, and R^{9′}, R^{9″}, R^{10′}, and R^{10″} are each hydrogen.

In certain embodiments, R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, —OC(O)R²⁰; and C₁₋₆ alkyl, optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In certain embodiments, R¹¹ and R¹² are independently selected from hydrogen, halogen, —OR²⁰, —SR²⁰, —N(R²⁰)₂, —C(O)R²⁰; and C₁₋₆ alkyl, optionally substituted with one or more substituents independently selected from halogen, —OR²⁰, —SR²⁰, and 3- to 12-membered heterocycle. In certain embodiments, R¹¹ and R¹² are independently selected from C₁₋₆ alkyl, substituted with one or more substituents independently selected from halogen, —OR²⁰, —SR²⁰, and 3- to 12-membered heterocycle. In certain embodiments, R¹¹ and R¹² are independently selected from C₁₋₆ alkyl substituted with one or more substituents independently selected from 3- to 12-membered heterocycle.

In certain embodiments, R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In certain embodiments, R¹³ and R¹⁴ are independently selected from hydrogen, halogen, —OR²⁰, —SR²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In certain embodiments, R¹³ and R¹⁴ are independently selected from C₁₋₆ alkyl optionally substituted with one or more substituents independently selected from halogen, —OC(O)R²⁰, C₃₋₁₂ carbocycle, and 3- to 12-membered heterocycle. In certain embodiments, R¹³ and R¹⁴ are independently selected from C₁₋₆ alkyl substituted with one or more substituents independently selected from halogen and 3- to 12-membered heterocycle.

In certain embodiments, R³ and R¹¹ taken together form an optionally substituted 5- to 6-membered heterocycle. In certain embodiments, R¹¹ and R¹² taken together form an optionally substituted C₃₋₆ carbocycle. In certain embodiments, X² is C(O).

In certain embodiments, a compound of Formula (IA), (IB), or (IC) is represented by the structure:

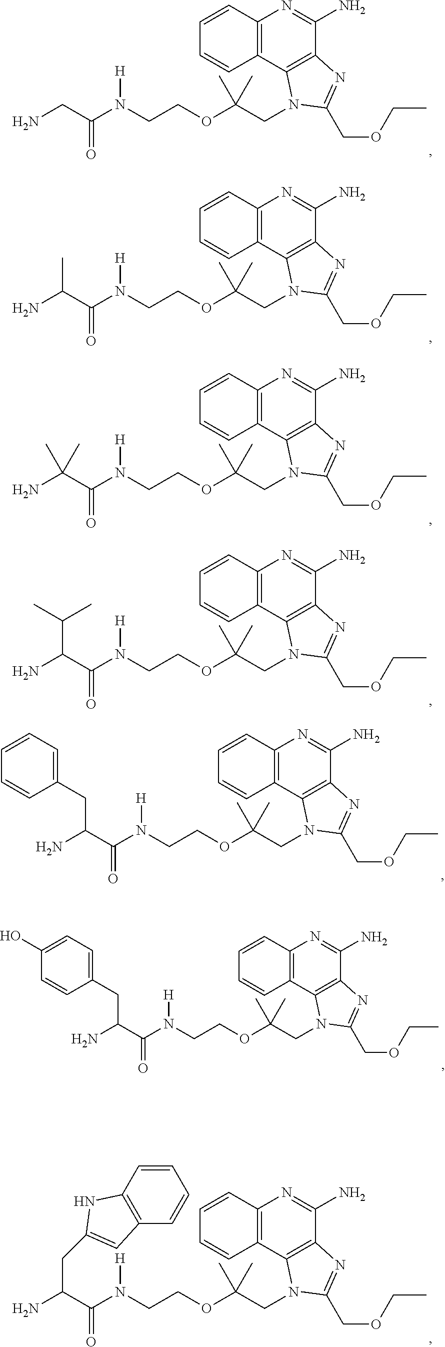

or a salt of any one thereof.

In certain embodiments, a pharmaceutical composition comprises the compound or salt of a compound of Formula (IA), (IB), or (IC), and a pharmaceutically acceptable excipient. In certain embodiments, a compound of Formula (IA), (IB), or (IC) or a salt of any one thereof, is further covalently bound to a linker, L³.

In certain aspects, compounds or conjugates of the disclosure are administered in a masked form suitable to attenuate or eliminate immune-modulatory activity of the compound or conjugate until the compound or conjugate reaches a desired target and the active site amine is unmasked. While not wishing to be bound by a mechanistic theory, the modification of compounds to attenuate or eliminate immune-modulatory activity may prevent undesired off-target immune-stimulatory activity, e.g., immune-stimulation in healthy tissue.

In certain embodiments, a compound such as a TLR7 agonist is modified with a masking group, such that the TLR7 agonist has limited activity or is inactive until it reaches an environment where the masking group is removed to reveal the active compound. For example, the TLR7 agonist is covalently modified at an amine involved in binding to the active site of a TLR7 receptor such that the compound is unable to bind the active site of the receptor in its modified form. In such an example, the masking group may be removed under physiological conditions, e.g., enzymatic or acidic conditions, specific to the site of delivery, e.g., intracellular or extracellular adjacent to target cells. In certain embodiments, the amine masking group inhibits binding of the amine group of the compound with residues of a TLR7 receptor. The amine masking group is removable under physiological conditions within a cell but remains covalently bound to the amine outside of a cell. Masking groups that may be used to inhibit or attenuate binding of an amine group of a compound with residues of a TLR7 receptor include, for example, peptides and carbamates.

In some embodiments for a compound or salt of Formula (VIIA), (VIIB), (VIIC) or the compound-linker constructs of Formula (VIIIA), (VIIIB), or (VIIIC), R⁵² is an amine masking group that is enzymatically-labile under in vivo promoiety. In certain embodiments, R⁵² is represented by the formula:

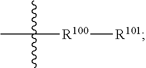

wherein:

R¹⁰¹ is selected from an amino acid, a peptide, —O—(C₁-C₆ alkyl) and —C₁-C₆ alkyl, wherein alkyl of —O—(C₁-C₆ alkyl) and —C₁-C₆ alkyl is optionally substituted by one or more substituents independently selected from halogen, —OR¹⁰, —SR¹⁰, —N(R¹⁰)₂, —C(O)R¹⁰, —C(O)N(R¹⁰)₂, —NO₂, —CN, C₃₋₁₃ carbocycle, and 3- to 12-membered heterocycle; and

R¹⁰⁰ is C(═O), wherein when R¹⁰¹ is selected from an amino acid or peptide R¹⁰⁰ is the C-terminus of the amino acid or peptide.

In certain embodiments, R¹⁰¹ of R⁵² is a peptide selected from a dipeptide, tripeptide and tetrapeptide.

In certain embodiments, R⁵² is selected from a group having a bond to an amine that is selectively cleaved under intracellular conditions

In certain embodiments, R¹⁰¹ is selected from —O—(C₁-C₄ alkyl) and —C₁-C₄ alkyl, wherein alkyl of —O—(C₁-C₄ alkyl) and —C₁-C₄ alkyl is optionally substituted by one or more substituents independently selected from halogen, —OR¹⁰, —SR¹⁰, —N(R¹⁰)₂, —C(O)R¹⁰, —C(O)N(R¹⁰)₂, —NO₂, —CN, C₃₋₁₃ carbocycle, and 3- to 12-membered heterocycle. In certain embodiments, R² is selected from 9-fluorenylmethylcarbonyl-, tert-butoxycarbonyl-, benzyloxycarbonyl-, acetyl-, and trifluoroacetyl-.

In certain embodiments, the amino acid of R¹⁰¹ is selected from any natural or non-natural amino acid. The amino acid may be selected from arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. In certain embodiments, the amino acid is an L-amino acid.

In certain embodiments, the peptide of R¹⁰¹ includes amino acids each independently selected from any natural or non-natural amino acid. The first amino acid (including R¹⁰⁰) may each be independently selected from arginine, histidine, lysine, aspartic acid, glutamic acid, serine, threonine, asparagine, glutamine, cysteine, selenocysteine, glycine, alanine, valine, isoleucine, leucine, methionine, phenylalanine, tyrosine, and tryptophan. In certain embodiments, the amino acids are each independently L-amino acids or D-amino acids. In certain embodiments, the peptide is a dipeptide, tripeptide or tetrapeptide. In certain embodiments, each amino acid of a dipeptide, tripeptide or tetrapeptide, is independently selected from a D- and L-amino acid. In certain embodiments, the amino acid immediately attached to the amine is an L-amino acid, e.g., R¹⁰¹ is represented by the formula: -aa1-aa2, or -aa1-aa2-aa3, where aa1 is an L-amino acid and aa2 and aa3 are independently selected from D- and L-amino acids. In certain embodiments, the first amino acid (including R¹⁰⁰) is an L-amino acid selected from arginine, histidine, lysine, aspartic acid, glutamic acid, serine, threonine, asparagine, glutamine, cysteine, selenocysteine, glycine, alanine, valine, isoleucine, leucine, methionine, phenylalanine, tyrosine, and tryptophan and the remaining amino acids are D or L amino acids selected from arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan.

In certain embodiments, an amine masking group is selected from those described in Protective Groups in Organic Synthesis (T. W. Green, P. G. M. Wuts, Wiley-Intersience, N Y, 1999).

In some aspects, the present disclosure provides a compound represented by Formula (VIIA):

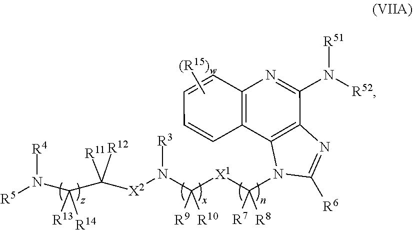

or a salt thereof, wherein:

R⁵¹ is hydrogen;

R⁵² is an amine masking group;

R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In certain embodiments, the present disclosure provides a compound or salt of Formula (VIIA), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

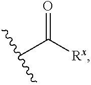

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In some aspects, the present disclosure provides a compound represented by Formula (VIIB).

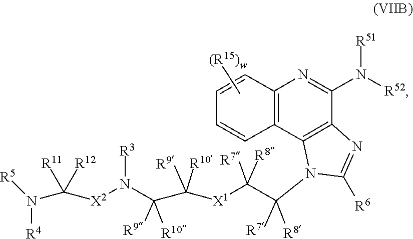

or a salt thereof, wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

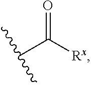

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, 2, 3, or 4.

In certain embodiments, the present disclosure provides a compound or salt of Formula (VIIB), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

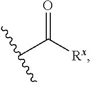

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R³, R⁴, and R⁵ are independently selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, or 2.

In certain embodiments, the present disclosure provides a compound or salt of Formula (VIIB), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

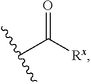

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R³, R⁴, and R⁵ are independently selected from hydrogen; and C₁₋₆ alkyl, optionally substituted with one or more substituents independently selected from halogen, —OR²⁰, —SR²⁰, —N(R²⁰)₂, and —CN;

R⁶ is selected from halogen and C₁₋₆ alkyl, optionally substituted with one or more substituents independently selected from halogen, —OR²⁰, —SR²⁰, and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen;

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, or 2.

In some aspects, the present disclosure provides a compound represented by Formula (VIIC):

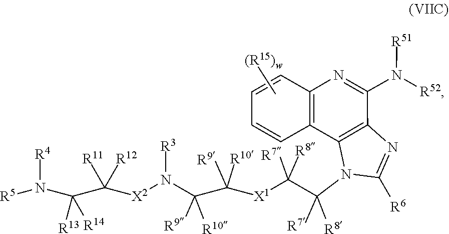

or a salt thereof, wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

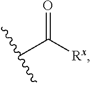

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, and —CN; C₁₋₆ alkyl, optionally substituted with one or more substituents independently selected from halogen, —OR²⁰, —SR²⁰, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, 2, 3, or 4.

In certain embodiments, the present disclosure provides a compound or salt of Formula (VIIC), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

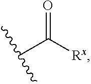

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R³, R⁴, and R⁵ are independently selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁶ is selected from halogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹³ and R¹⁴ are independently selected at each occurrence from hydrogen, halogen, —OR²⁰, —SR²⁰, and —CN; C₁₋₆ alkyl, optionally substituted with one or more substituents independently selected from halogen, —OR²⁰, —SR²⁰, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, or 2.

In certain embodiments, the present disclosure provides a compound or salt of Formula (VIIC), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

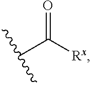

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R³, R⁴, and R⁵ are independently selected from hydrogen; and C₁₋₆ alkyl, optionally substituted with one or more substituents independently selected from halogen, —OR²⁰, —SR²⁰, —N(R²⁰)₂, and —CN;

R⁶ is selected from halogen and C₁₋₆ alkyl, optionally substituted with one or more substituents independently selected from halogen, —OR²⁰, —SR²⁰, and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen;

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹³ and R¹⁴ are independently selected at each occurrence from hydrogen, halogen, —OR²⁰, —SR²⁰, and —CN; C₁₋₆ alkyl, optionally substituted with one or more substituents independently selected from halogen, —OR²⁰, —SR²⁰, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen;

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

X¹ is O, S, or NR¹⁶;

X² is C(O) or S(O)₂; and

w is 0, 1, or 2.

In an aspect, the present disclosure provides a compound represented by Formula (IIA):

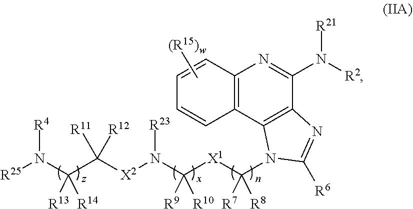

or a salt thereof, wherein:

R² and R⁴ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²¹, R²³, and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, —CN, C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle; L³ is a linker;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In certain embodiments, X¹ is 0. In certain embodiments, n is 2. In certain embodiments, x is 2. In certain embodiments, z is 0. In certain embodiments, z is 1.

In certain embodiments, the compound of Formula (IIA) is represented by (IIB) or (IIC):

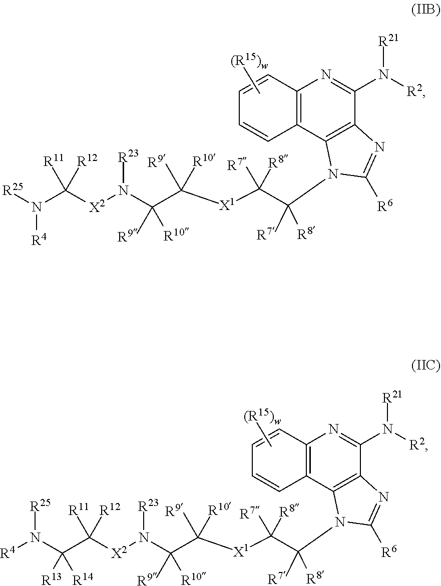

or a salt thereof, wherein:

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In certain embodiments, the present disclosure provides a compound or salt of Formula (IIB), wherein:

R² and R⁴ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²¹, R²³, and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle; L³ is a linker;

X¹ is O, S, or NR¹⁶;

X² is C(O) or S(O)₂; and

w is 0, 1, 2, 3, or 4.

In certain embodiments, the present disclosure provides a compound or salt of Formula (IIB), wherein:

R² and R⁴ are independently selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; wherein one of R²¹, R²³, and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₃₋₁₂ carbocycle, and 3- to 12-membered heterocycle; L³ is a linker;

X¹ is O, S, or NR¹⁶;

X² is C(O) or S(O)₂; and

w is 0, 1, 2, 3, or 4.

In certain embodiments, the present disclosure provides a compound or salt of Formula (IIB), wherein:

R² and R⁴ are independently selected from hydrogen; and C₁₋₆ alkyl, optionally substituted with one or more substituents independently selected from halogen, —OR²⁰, —SR²⁰ and —CN;

R²¹, R²³, and R²⁵ are independently selected from hydrogen; C₁₋₆ alkyl, optionally substituted with one or more substituents independently selected from halogen, —OR²⁰, —SR²⁰, —N(R²⁰)₂, and —CN; and L³; wherein one of R²¹, R²³, and R²⁵ is L³;

R⁶ is selected from halogen, —OR²⁰, —N(R²⁰)₂, and C₁₋₆ alkyl, optionally substituted with one or more substituents independently selected from halogen, —OR²⁰, —SR²⁰, and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —CN, C₃₋₁₂ carbocycle, and 3- to 12-membered heterocycle;

R¹⁵ is independently selected at each occurrence from halogen, —OR²⁰, —SR²⁰, —N(R²⁰)₂, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₃₋₁₂ carbocycle, and 3- to 12-membered heterocycle; L³ is a linker;

X¹ is O, S, or NR¹⁶;

X² is C(O); and

w is 0, 1, or 2.

In certain embodiments, the present disclosure provides a compound or salt of Formula (IIC), wherein:

R² and R⁴ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²¹, R²³, and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, —CN, C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle; L³ is a linker;

X¹ is O, S, or NR¹⁶;

X² is C(O) or S(O)₂; and

w is 0, 1, 2, 3, or 4.

In certain embodiments, the present disclosure provides a compound or salt of Formula (IIC), wherein:

R² and R⁴ are independently selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; wherein one of R²¹, R²³, and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹³ and R¹⁴ are independently selected at each occurrence from hydrogen, halogen, —OR²⁰, —SR²⁰, —CN, C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₃₋₁₂ carbocycle, and 3- to 12-membered heterocycle; L³ is a linker;

X¹ is O, S, or NR¹⁶;

X² is C(O) or S(O)₂; and

w is 0, 1, 2, 3, or 4.

In certain embodiments, the present disclosure provides a compound or salt of Formula (IIC), wherein:

R² and R⁴ are independently selected from hydrogen; and C₁₋₆ alkyl, optionally substituted with one or more substituents independently selected from halogen, —OR²⁰, —SR²⁰ and —CN;

R²¹, R²³, and R²⁵ are independently selected from hydrogen; C₁₋₆ alkyl, optionally substituted with one or more substituents independently selected from halogen, —OR²⁰, —SR²⁰, —N(R²⁰)₂, and —CN; and L³; wherein one of R²¹, R²³, and R²⁵ is L³;

R⁶ is selected from halogen, —OR²⁰, —N(R²⁰)₂, and C₁₋₆ alkyl, optionally substituted with one or more substituents independently selected from halogen, —OR²⁰, —SR²⁰, and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —CN, C₃₋₁₂ carbocycle, and 3- to 12-membered heterocycle;

R¹³ and R¹⁴ are independently selected at each occurrence from hydrogen, halogen, —OR²⁰, —SR²⁰, —CN, C₁₋₆ alkyl, optionally substituted with one or more substituents independently selected from halogen, —OR²⁰, —SR²⁰, —N(R²⁰)₂, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —N(R²⁰)₂, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₃₋₁₂ carbocycle, and 3- to 12-membered heterocycle; L³ is a linker;

X¹ is O, S, or NR¹⁶;

X² is C(O); and

w is 0, 1, or 2.

In certain embodiments, R² and R⁴ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In certain embodiments, R² and R⁴ are independently selected from hydrogen and C₁₋₆ alkyl. In certain embodiments, R² and R⁴ are each hydrogen. In certain embodiments, R²³ is selected from hydrogen and C₁₋₆ alkyl optionally substituted with one or more halogens. In certain embodiments, R²³ is hydrogen. In certain embodiments, R²¹ is selected from hydrogen and C₁₋₆ alkyl optionally substituted with one or more halogens. In certain embodiments, R²¹ is hydrogen. In certain embodiments, R²¹ is L³. In certain embodiments, R²⁵ is selected from hydrogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In certain embodiments, R²⁵ is hydrogen. In certain embodiments, R²⁵ is L³.

In certain embodiments, for a compound or salt of Formula (IIA), (JIB), or (IIC), R² and R⁴ are each hydrogen, R²¹ is L³, R²³ is selected from hydrogen and C₁₋₆ alkyl, and R²⁵ is selected from hydrogen and C₁₋₆ alkyl. In certain embodiments, for a compound or salt of Formula (IIA), (IIB), or (IIC), R² and R⁴ are each hydrogen, R²¹ is selected from hydrogen and C₁₋₆ alkyl, R²³ is L³, and R²⁵ is selected from hydrogen and C₁₋₆ alkyl. In certain embodiments, for a compound or salt of Formula (IIA), (IIB), or (IIC), R² and R⁴ are each hydrogen, R²¹ is selected from hydrogen and C₁₋₆ alkyl, R²³ is selected from hydrogen and C₁₋₆ alkyl, and R²⁵ is L³.

In certain embodiments, for a compound or salt of Formula (IIA), (IIB), or (IIC), R⁶ is selected from halogen, —OR²⁰, and —N(R²⁰)₂; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle. In certain embodiments, R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and R²⁰ is independently selected at each occurrence from hydrogen, —NH₂, —C(O)OCH₂C₆H₅; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle. In certain embodiments, R⁶ is C₁₋₆ alkyl substituted with —OR²⁰, and R²⁰ is selected from hydrogen and C₁₋₆ alkyl, which is optionally substituted with one or more substituents independently selected from halogen, —OH, and —NH₂.

In certain embodiments, R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl optionally substituted with one or more substituents independently selected from halogen. In certain embodiments, R⁷ is hydrogen. In certain embodiments, R^{7′} is hydrogen. In certain embodiments, R⁸ is hydrogen. In certain embodiments, R^{8′} is hydrogen. In certain embodiments, R^{7″} and R^{8″} are C₁₋₆ alkyl. In certain embodiments, R^{7″} and R^{8″} are methyl.

In certain embodiments, R^{9′}, R^{9″}, R^{10′}, and R^{10″} are independently selected at each occurrence from hydrogen and C₁₋₆ alkyl. In certain embodiments, R^{9′}, R^{9″}, R^{10′}, and R^{10″} are independently selected at each occurrence from hydrogen and methyl, ethyl, and propyl. In certain embodiments, R^{9′}, R^{9″}, R^{10′}, and R^{10″} are each hydrogen.

In certain embodiments, R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In certain embodiments, R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl. In certain embodiments, R¹¹ and R¹² are independently selected from hydrogen and halogen.

In certain embodiments, R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle. In certain embodiments, R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl. In certain embodiments, R¹³ and R¹⁴ are independently selected from hydrogen, halogen, and C₁₋₆ alkyl.

In certain embodiments, R²³ and R¹¹ taken together form an optionally substituted 5- to 6-membered heterocycle. In certain embodiments, R¹¹ and R¹² taken together form an optionally substituted C₃₋₆ carbocycle. In certain embodiments, X² is C(O).

In an aspect, the present disclosure provides a compound represented by Formula (VIIIA):

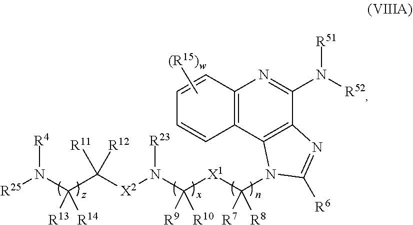

or a salt thereof, wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

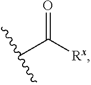

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁴ is selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²³ and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²³ and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, —CN, C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

L³ is a linker;

X¹ is O, S, or NR¹⁶;

X² is C(O) or S(O)₂;

n is 1, 2, or 3;

x is 1, 2, or 3;

w is 0, 1, 2, 3, or 4; and

z is 0, 1, or 2.

In certain embodiments, X¹ is 0. In certain embodiments, n is 2. In certain embodiments, x is 2. In certain embodiments, z is 0. In certain embodiments, z is 1.

In certain embodiments, the compound of Formula (VIIIA) is represented by (VIIIB) or (VIIIC):

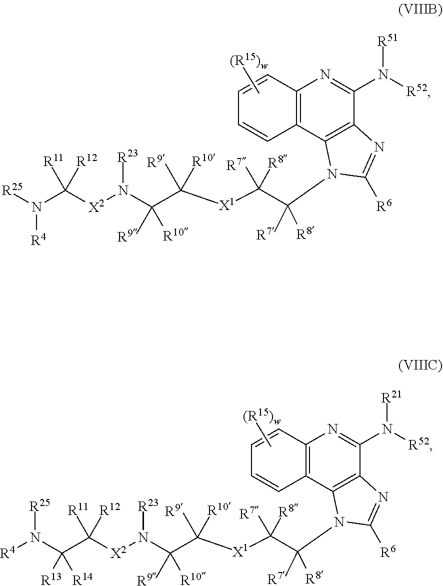

or a salt thereof, wherein.

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In certain embodiments, the present disclosure provides a compound or salt of Formula (VIIIB), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

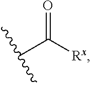

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁴ is selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²³ and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²³ and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

L³ is a linker;

X¹ is O, S, or NR¹⁶;

X² is C(O) or S(O)₂; and

w is 0, 1, 2, 3, or 4.

In certain embodiments, the present disclosure provides a compound or salt of Formula (VIIIB), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

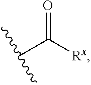

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁴ is selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²³ and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; wherein one of R²³ and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₃₋₁₂ carbocycle, and 3- to 12-membered heterocycle;

L³ is a linker;

X¹ is O, S, or NR¹⁶;

X² is C(O) or S(O)₂; and

w is 0, 1, 2, 3, or 4.

In certain embodiments, the present disclosure provides a compound or salt of Formula (VIIIB), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

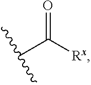

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁴ is selected from hydrogen; and C₁₋₆ alkyl, optionally substituted with one or more substituents independently selected from halogen, —OR²⁰, —SR²⁰, and —CN;

R²³ and R²⁵ are independently selected from hydrogen; C₁₋₆ alkyl, optionally substituted with one or more substituents independently selected from halogen, —OR²⁰, —SR²⁰, —N(R²⁰)₂, and —CN; and L³; wherein one of R²³ and R²⁵ is L³;

R⁶ is selected from halogen, —OR²⁰, —N(R²⁰)₂, and C₁₋₆ alkyl, optionally substituted with one or more substituents independently selected from halogen, —OR²⁰, —SR²⁰, and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —CN, C₃₋₁₂ carbocycle, and 3- to 12-membered heterocycle;

R¹⁵ is independently selected at each occurrence from halogen, —OR²⁰, —SR²⁰, —N(R²⁰)₂, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₃₋₁₂ carbocycle, and 3- to 12-membered heterocycle;

L³ is a linker;

X¹ is O, S, or NR¹⁶;

X² is C(O); and

w is 0, 1, or 2.

In certain embodiments, the present disclosure provides a compound or salt of Formula (VIIIC), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

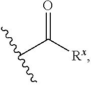

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁴ is selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²³, and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, —CN, C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

L³ is a linker;

X¹ is O, S, or NR¹⁶;

X² is C(O) or S(O)₂; and

w is 0, 1, 2, 3, or 4.

In certain embodiments, the present disclosure provides a compound or salt of Formula (VIIIC), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

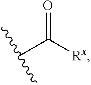

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁴ is selected from hydrogen; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R²³ and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; wherein one of R²³ and R²⁵ is L³;

R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle;

R¹³ and R¹⁴ are independently selected at each occurrence from hydrogen, halogen, —OR²⁰, —SR²⁰, —CN, C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₃₋₁₂ carbocycle, and 3- to 12-membered heterocycle;

L³ is a linker;

X¹ is O, S, or NR¹⁶;

X² is C(O) or S(O)₂; and

w is 0, 1, 2, 3, or 4.

In certain embodiments, the present disclosure provides a compound or salt of Formula (VIIIC), wherein:

R⁵¹ is hydrogen;

R⁵² is an amino acid, di-peptide, tri-peptide, or tetra-peptide, wherein the point of attachment to the nitrogen is the C-terminus of the amino acid, di-peptide, tri-peptide, or tetra-peptide,

or R⁵² is represented by the formula:

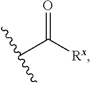

wherein R^{x} is selected from optionally substituted C₁₋₆ alkyl, —O-(optionally substituted C₁₋₆ alkyl) wherein optional substituents are selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;

R⁴ is selected from hydrogen; and C₁₋₆ alkyl, optionally substituted with one or more substituents independently selected from halogen, —OR²⁰, —SR²⁰, and —CN;

R²³ and R²⁵ are independently selected from hydrogen; C₁₋₆ alkyl, optionally substituted with one or more substituents independently selected from halogen, —OR²⁰, —SR²⁰, —N(R²⁰)₂, and —CN; and L³; wherein one of R²³ and R²⁵ is L³;

R⁶ is selected from halogen, —OR²⁰, —N(R²⁰)₂, and C₁₋₆ alkyl, optionally substituted with one or more substituents independently selected from halogen, —OR²⁰, —SR²⁰, and —CN;

R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

R¹¹ and R¹² are independently selected from hydrogen, halogen, —OR²⁰, —SR²⁰, and —CN; and C₁₋₆ alkyl, optionally substituted with one or more substituents independently selected from halogen, —OR²⁰, —SR²⁰, —CN, C₃₋₁₂ carbocycle, and 3- to 12-membered heterocycle;

R¹³ and R¹⁴ are independently selected at each occurrence from hydrogen, halogen, —OR²⁰, —SR²⁰, —CN, C₁₋₆ alkyl, optionally substituted with one or more substituents independently selected from halogen, —OR²⁰, —SR²⁰, —N(R²⁰)₂, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, ═O, ═S, ═N(R²⁰), —CN, C₁₋₆ alkyl, C₂₋₆ alkenyl, and C₂₋₆ alkynyl;

R¹⁵ is independently selected at each occurrence from halogen, —OR²⁰, —SR²⁰, —N(R²⁰)₂, C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R¹⁶ is selected from hydrogen; and C₁₋₆ alkyl, optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;

R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₃₋₁₂ carbocycle, and 3- to 12-membered heterocycle;

L³ is a linker;

X¹ is O, S, or NR¹⁶;

X² is C(O); and

w is 0, 1, or 2.

In certain embodiments, L³ is a cleavable linker. In certain embodiments, L³ is cleavable by a lysosomal enzyme. In certain embodiments, L³ is represented by the formula:

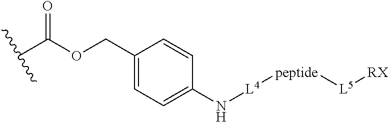

wherein: L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and R³⁰ is independently selected at each occurrence from halogen, —OH, —CN, —O-alkyl, —SH, ═O, ═S, —NH₂, —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂. In certain embodiments, RX comprises a leaving group. In certain embodiments, RX is a maleimide or an alpha-halo carbonyl. In certain embodiments, the peptide of L³ comprises Val-Cit or Val-Ala. In certain embodiments, L³ is represented by the formula:

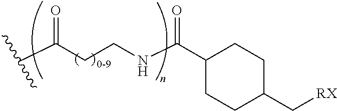

wherein: RX comprises a reactive moiety; and n is 0-9. In certain embodiments, RX comprises a leaving group. In certain embodiments, RX is a maleimide or an alpha-halo carbonyl. In certain embodiments, L³ is further covalently bound to an antibody construct to form a conjugate.

In some embodiments, the present disclosure provides a conjugate represented by the formula:

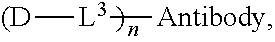

wherein: Antibody is an antibody construct; n is 1 to 20; D is the compound or salt of a Formula herein; and L³ is a linker moiety. In certain embodiments, n is selected from 1 to 8. In certain embodiments, n is 1, 2, 3, 4, 5, 6, 7, or 8. In certain embodiments, n is 2, 3, 4, 5, or 6. In certain embodiments, n is selected from 2 to 5. In certain embodiments, n is 2, 3, or 4. In certain embodiments, n is 2. In certain embodiments, -L³ is represented by the formula:

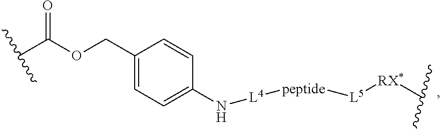

wherein: L⁴ represents the C-terminus of the peptide and L⁵ is selected from a bond, alkylene and heteroalkylene, wherein L⁵ is optionally substituted with one or more groups independently selected from R³⁰; RX* is a bond, a succinimide moiety, or a hydrolyzed succinimide moiety bound to a residue of an antibody construct, wherein [symbol] on RX* represents the point of attachment to the residue of the antibody construct; and R³⁰ is independently selected at each occurrence from halogen, —OH, —CN, —O— alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂. In certain embodiments, RX* is a succinamide moiety, hydrolyzed succinamide moiety or a mixture thereof and is bound to a cysteine residue of an antibody construct. In certain embodiments, RX* is a succinamide moiety. In certain embodiments, RX* is a hydrolyzed succinamide moiety. In certain embodiments, -L³ is represented by the formula:

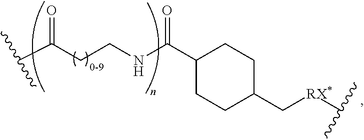

wherein: RX* is a bond, a succinimide moiety, or a hydrolyzed succinimide moiety bound to a residue of an antibody construct, wherein [symbol] on RX* represents the point of attachment to the residue of the antibody construct; and n is 0-9. In certain embodiments, the antibody construct comprises an antigen binding domain that specifically binds to an antigen selected from the group consisting of CD5, CD19, CD20, CD25, CD37, CD30, CD33, CD40, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), GD2, GD3, GM2, Le^{y}, CA-125, CA19-9, epidermal growth factor, p185HER2, IL-2 receptor, EGFRvIII (de2-7 EGFR), fibroblast activation protein, tenascin, a metalloproteinase, endosialin, vascular endothelial growth factor, avB3, WT1, LMP2, HPV E6, HPV E7, Her-2/neu, MAGE A3, p53 nonmutant, NY-ESO-1, MelanA/MART1, Ras mutant, gp100, p53 mutant, PR1, bcr-abl, tyronsinase, survivin, PSA, hTERT, a Sarcoma translocation breakpoint protein, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TESL Sperm protein 17, LCK, HMWMAA, AKAP-4, SSX2, XAGE 1, Legumain, Tie 3, VEGFR2, MAD-CT-1, PDGFR-B, MAD-CT-2, ROR2, TRAIL1, MUC16, MAGE A4, MAGE C2, GAGE, EGFR, CMET, HER3, MUC15, CA6, NAPI2B, TROP2, CLDN6, CLDN16, CLDN18.2, CLorfl86, RON, LY6E, FRA, DLL3, PTK7, STRA6, TMPRSS3, TMPRSS4, TMEM238, UPK1B, VTCN1, LIV1, ROR1, or Fos-related antigen 1, and preferably Her2. In certain embodiments, the present disclosure provides a pharmaceutical composition, comprising a conjugate disclosed herein, and a pharmaceutically acceptable excipient. In certain embodiments, a compound disclosed herein may be referred to as a “drug.” In certain embodiments, a compound disclosed herein, e.g. a drug, may have a certain ratio to an antibody in a pharmaceutical composition. In certain embodiments, the ratio may be referred to as an average drug-to-antibody ratio. In certain embodiments, a pharmaceutical composition described herein may have an average Drug-to-Antibody Ratio (DAR) of 1 to 8. In certain embodiments, a pharmaceutical composition described herein may have an average Drug-to-Antibody Ratio (DAR) of 2 to 6. In certain embodiments, a pharmaceutical composition described herein may have an average Drug-to-Antibody Ratio (DAR) of 1 to 5. In certain embodiments, a pharmaceutical composition described herein may have an average Drug-to-Antibody Ratio (DAR) of 3 to 5. In certain embodiments, a pharmaceutical composition described herein may have an average Drug-to-Antibody Ratio (DAR) of 2. In certain embodiments, a pharmaceutical composition described herein may have an average Drug-to-Antibody Ratio (DAR) of 2, 3, 4, 5, 6, or 7.

In some embodiments, the present disclosure provides a method for treating cancer, comprising administering an effective amount of a compound or salt disclosed herein or a pharmaceutical composition disclosed herein to a subject in need thereof. In some embodiments, the present disclosure provides a method for treating cancer, comprising administering an effective amount of a conjugate described herein, or a pharmaceutical composition described herein to a subject in need thereof

In some embodiments, the present disclosure provides a method of killing tumor cells in vivo, comprising contacting a tumor cell population with a conjugate described herein or a pharmaceutical composition described herein. In some embodiments, the present disclosure provides a method for treatment, comprising administering to a subject a conjugate described herein or a pharmaceutical composition described herein

In some embodiments, a compound or salt described herein, or a pharmaceutical composition described herein, may be used in a method of treatment of a subject's body by therapy. In some embodiments, a compound or salt described herein, or a pharmaceutical composition described herein, may be used in a method of treating cancer. In some embodiments, a conjugate described herein, or a pharmaceutical composition described herein, may be used in a method of treatment of a subject's body by therapy. In some embodiments, a conjugate described herein or a pharmaceutical composition described herein, may be used in a method of treating cancer.

In some embodiments, the present disclosure provides a method of preparing an antibody conjugate of the formula:

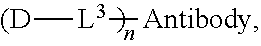

wherein: Antibody is an antibody construct; n is selected from 1 to 20; and
D-L³ is selected from a compound or salt for a Formula herein, comprising contacting D-L³ with an antibody construct. In some embodiments, the present disclosure provides a method of preparing an antibody conjugate of the formula:

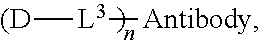

wherein: Antibody is an antibody construct; n is selected from 1 to 20; L³ is a linker; and D is selected from a compound or salt of a Formula herein, comprising contacting L³ with the antibody construct to form L³-antibody and contacting L³ antibody with D to form the conjugate. In some embodiments, the antibody construct comprises an antigen binding domain that specifically binds to an antigen selected from the group consisting of CD5, CD19, CD20, CD25, CD37, CD30, CD33, CD40, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), GD2, GD3, GM2, Le^{y}, CA-125, CA19-9, epidermal growth factor, p185HER2, IL-2 receptor, EGFRvIII (de2-7 EGFR), fibroblast activation protein, tenascin, a metalloproteinase, endosialin, vascular endothelial growth factor, avB3, WT1, LMP2, HPV E6, HPV E7, Her-2/neu, MAGE A3, p53 nonmutant, NY-ESO-1, MelanA/MART1, Ras mutant, gp100, p53 mutant, PR1, bcr-abl, tyronsinase, survivin, PSA, hTERT, a Sarcoma translocation breakpoint protein, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TESL Sperm protein 17, LCK, HMWMAA, AKAP-4, SSX2, XAGE 1, Legumain, Tie 3, VEGFR2, MAD-CT-1, PDGFR-B, MAD-CT-2, ROR2, TRAIL1, MUC16, MAGE A4, MAGE C2, GAGE, EGFR, CMET, HER3, MUC15, CA6, NAPI2B, TROP2, CLDN6, CLDN16, CLDN18.2, CLorfl86, RON, LY6E, FRA, DLL3, PTK7, STRA6, TMPRSS3, TMPRSS4, TMEM238, UPK1B, VTCN1, LIV1, ROR1, or Fos-related antigen 1, and preferably Her2. In some embodiments, the method further comprises purifying the antibody conjugate.

Any combination of the groups described above for the various variables is contemplated herein. Throughout the specification, groups and substituents thereof can be chosen to provide stable moieties and compounds.

In certain embodiments, a linker may comprise a reactive moiety, e.g., an electrophile, that can react to form a covalent bond with a moiety of an antibody construct such as, for example, a lysine, serine, threonine, cysteine, tyrosine, aspartic acid, glutamine, a non-natural amino acid residue, or glutamic acid residue of any antibody. In some embodiments, a compound or salt described herein may be covalently bound through the linker to an antibody construct.

In certain embodiments, a moiety described herein includes the symbol [symbol] which indicates the point of attachment, e.g., the point of attachment of a chemical moiety to the remainder of the compound, the point of attachment of a linker to a compound of the disclosure, or the point of attachment of a linker to an antibody construct, as described herein.

Some linkers (L³) are described in the following paragraphs and additional linkers are described in the subsequent section entitled “Linkers”. In some embodiments for a compound or salt disclosed herein, -L³ is represented by the formula:

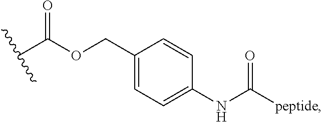

wherein peptide is a group comprising from one to ten amino acids.
In some embodiments for a compound or salt of Formula (IA), (IB), (IC), (VIIA), (VIIB), or (VIIC), -L³ is represented by the formula:

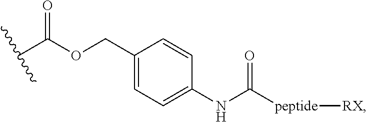

wherein peptide is a group comprising from one to ten amino acids and RX is a reactive moiety, and

[symbol]

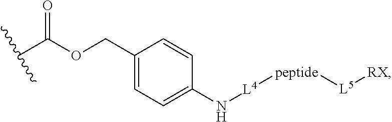

in which L⁴ represents the C-terminus of the peptide and L⁵ is selected from a bond, alkylene and heteroalkylene, wherein L⁵ is optionally substituted with one or more groups independently selected from R³², RX is a reactive moiety; and R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, and C₂₋₁₀ alkynyl, each of which is independently optionally substituted at each occurrence with one or more substituents selected from halogen, —OH, —CN, —O-alkyl, —SH, ═O, ═S, —NH₂, and —NO₂.

In certain embodiments, for a compound of any one of Formulas (IA), (IB), (IC), (VIIA), (VIIB), or (VIIC), -L³ is represented by the formula:

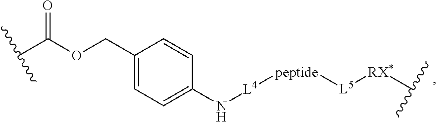

in which L⁴ represents the C-terminal of the peptide and L⁵ is selected from a bond, alkylene and heteroalkylene, wherein L⁵ is optionally substituted with one or more groups independently selected from R³²; RX* is a bond, a succinimide moiety, or a hydrolyzed succinimide moiety bound to a residue of an antibody construct, wherein [symbol] on RX* represents the point of attachment to the residue of the antibody construct; and, R³² is independently selected at each occurrence from halogen, —OH, —CN, —O— alkyl, —SH, ═O, ═S, —NH₂, —NO₂; and C₁₋₁₀ alkyl, C₂₋₁₀ alkenyl, and C₂₋₁₀ alkynyl, each of which is independently optionally substituted at each occurrence with one or more substituents selected from halogen, —OH, —CN, —O-alkyl, —SH, ═O, ═S, —NH₂, and —NO₂. The reactive moiety may be selected from an electrophile, e.g., an α,β-unsaturated carbonyl, such as a maleimide, and a leaving group.

In some embodiments for a compound or salt of Formula (IA), (IB), (IC), (VIIA), (VIIB), or (VIIC), -L³ is represented by the formula:

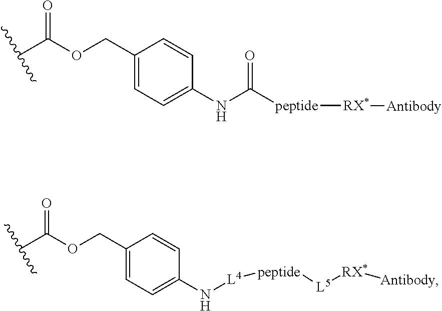

in which L⁴ represents the C-terminal of the peptide and L⁵ is selected from a bond, alkylene and heteroalkylene, wherein L⁵ is optionally substituted with one or more groups independently selected from R³²; RX* is a bond, a succinimide moiety, or a hydrolyzed succinimide moiety bound to a residue of an antibody construct, wherein Antibody is an antibody construct; and, R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, and C₂₋₁₀ alkynyl, each of which is independently optionally substituted at each occurrence with one or more substituents selected from halogen, —OH, —CN, —O-alkyl, —SH, ═O, ═S, —NH₂, and —NO₂. L³ may be represented by the formula:

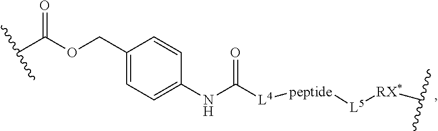

wherein L⁴ and L⁵ are independently selected from a bond, an alkylene and a heteroalkylene, each of which is optionally substituted with one or more groups independently selected from R¹²; [symbol] on the left represents the point of attachment to the remainder of the compound, RX* is a bond, a succinimide moiety, or a hydrolyzed succinimide moiety attached at the [symbol] the right to a residue of an antibody construct; peptide is a group comprising from one to 10 amino acids.

In some embodiments for a compound or salt of Formula (IA), (IB), (IC), (VIIA), (VIIB), or (VIIC), -L³ is represented by the formula:

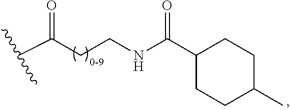

and [symbol] represents the point of attachment to the remainder of the compound.

In some embodiments for a compound or salt of Formula (IA), (IB), (IC), (VIIA), (VIIB), or (VIIC), -L³ is represented by the formula:

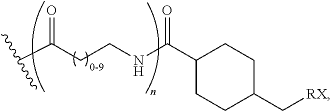

wherein RX comprises a reactive moiety, e.g., a maleimide or a leaving group, n=0-9 and [symbol] represents the point of attachment to the remainder of the compound.

In some embodiments, for a compound or salt of Formula (IA), (IB), (IC), (VIIA), (VIIB), or (VIIC), -L³ is represented by the formula:

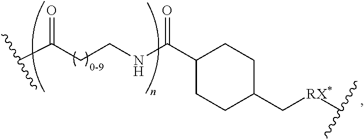

RX* is a bond, a succinimide moiety, or a hydrolyzed succinimide moiety attached at the [symbol] on the right to a residue of an antibody construct, n=0-9 and [symbol] on the left represents the point of attachment to the remainder of the compound.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

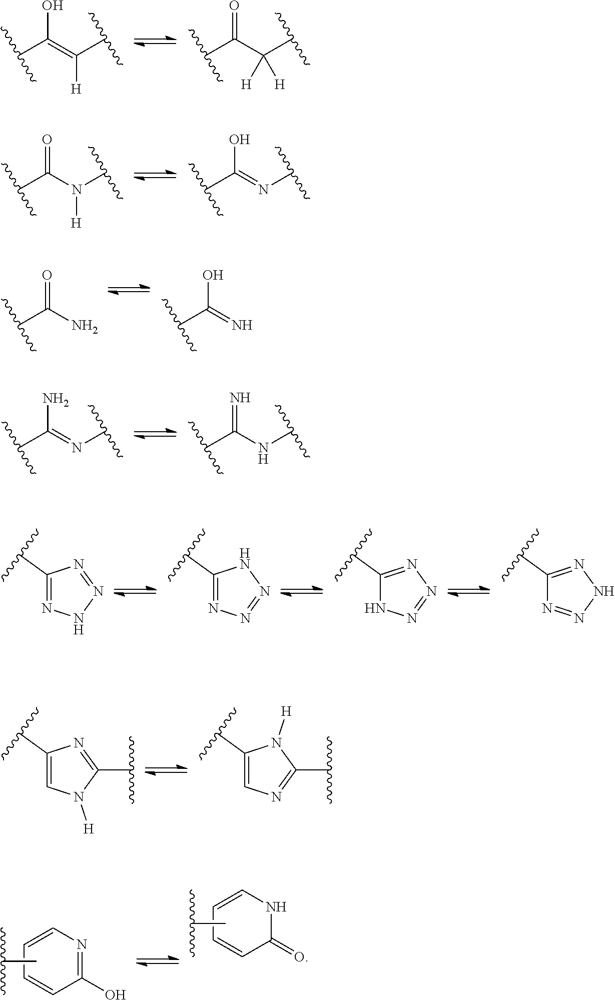

The compounds disclosed herein, in some embodiments, are used in different enriched isotopic forms, e.g., enriched in the content of ²H, ³H, ¹¹C, ¹³C and/or ¹⁴C. In one embodiment, the compound is deuterated in at least one position.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present disclosure, whether radioactive or not, are encompassed within the scope of the present disclosure.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

In some aspects, the present disclosure provides a method for treating cancer. In some embodiments, the present disclosure provides a method comprising administering a conjugate, compound or salt of Formula (IIA), (IIB), (IIC), (VIIIA), (VIIIB), or (VIIIC), or of Formula (IA), (IB), (IC), (VIIA), (VIIB), or (VIIC), respectively, to a subject in need thereof.

In some aspects, the compounds of the disclosure exhibit selective binding or agonizing properties for one receptor over another receptor. In some embodiments, a compound described herein selectively binds or modulates the activity of one toll-like receptor over another, e.g., TLR7 and TLR8.

In certain embodiments, a compound of the disclosure agonizes TLR7 with an EC₅₀ of 500 nM or less while the same compound agonizes TLR8 with an ED₅₀ of greater than 1 μM. In certain embodiments, a compound of the disclosure agonizes TLR7 with at least an EC₅₀ of an order of magnitude or even two orders of magnituge less than the amount of the same compound required to show an agonizing effect on TLR8. In some embodiments, a compound or salt of Formula (IA), (IB), (IC), (VIIA), (VIIB), or (VIIC) may have weaker binding affinity to TLR8 as compared to TLR7, as measured by the K_{d} values, e.g., a compound's K_{d} for TLR8 is two times or greater than two times the K_{d} for TLR7, or an order of magnitude or greater, or even two orders of magnitude or greater than the K_{d} for TLR7. In certain embodiments, a compound or salt of Formula (IA), (IB), (IC), (VIIA), (VIIB), or (VIIC) has greater activity for one toll-like receptor over another, e.g., TLR7 and TLR8.

Included in the present disclosure are salts, such as pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds of any one of Formulas (IA), (IB), (IC), (VIIA), (VIIB), or (VIIC) may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) may be prodrugs of the present disclosure.

Prodrug forms of the herein described compounds, wherein the prodrug is metabolized in vivo to produce a compound of any one of Formulas (IA), (IB), (IC), for example, as set forth herein are included within the scope of the claims. In some cases, some of the herein-described compounds may be a prodrug for another derivative or active compound. In certain embodiments, a prodrug of the disclosure may be selected from an amine masking group. The amine masking group is removable under physiological conditions within a cell but remains covalently bound to the amine outside of a cell. Masking groups that may be used to inhibit or attenuate binding of an amine group of a compound with residues of a TLR7 receptor include, for example, peptides and carbamates.

Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. According to another embodiment, the present disclosure provides methods of making the compounds described herein.

### Linkers

The compounds and salts described herein may be bound to a linker also referred to herein as L³, e.g., a peptide linker. In certain embodiments, the linker is also bound to an antibody construct and referred to as an antibody construct conjugate or conjugate. Linkers of the conjugates described herein may not affect the binding of active portions of a conjugate, e.g., the antigen binding domains, Fc domains, target binding domains, antibodies, agonists or the like, to a target, which can be a cognate binding partner such as an antigen. A conjugate can comprise multiple linkers, each having one or more compounds attached. These linkers can be the same linkers or different linkers. Some examplary linkers (L³) are described in the preceding section entitled “Compounds” and additional linkers are described in this section.

A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable or selectively stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker may comprise a valine-citrulline linker or a valine-alanine peptide. A valine-citrulline- or valine-alanine-containing linker can contain a pentafluorophenyl group. A valine-citrulline- or valine-alanine-containing linker can contain a maleimide or succinimide group. A valine-citrulline- or valine-alanine-containing linker can contain a para aminobenzyl alcohol (PABA) group or para-aminobenzyl carbamate (PABC).

A valine-citrulline- or valine-alanine-containing linker can contain a PABA group and a pentafluorophenyl group. A valine-citrulline- or valine-alanine-containing linker can contain a PABA group and a maleimide or succinimide group.

A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonly) linker. A linker can be a linker suitable for attachment to an engineered cysteine (THIOMAB). A THIOMAB linker can be a (maleimidocaproyl)-(valine-citrulline)-(para-aminobenzyloxycarbonly)-linker.

A linker can also comprise alkylene, alkenylene, alkynylene, polyether, polyester, polyamide group(s) and also, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif to an N-terminal GGG motif to regenerate a native amide bond. The linker created can therefore link a moiety attached to the LXPTG recognition motif with a moiety attached to the N-terminal GGG motif.

In the conjugates described herein, a compound or salt of any one of Formulas (IA), (IB), (IC), (VIIA), (VIIB), or (VIIC) is linked to the antibody construct by way of a linker(s), also referred to herein as L³. L³, as used herein, may be selected from any of the linker moieties discussed herein. The linker linking the compound or salt to the antibody construct of a conjugate may be short, long, hydrophobic, hydrophilic, flexible or rigid, or may be composed of segments that each independently have one or more of the above-mentioned properties such that the linker may include segments having different properties. The linkers may be polyvalent such that they covalently link more than one compound or salt to a single site on the antibody construct, or monovalent such that covalently they link a single compound or salt to a single site on the antibody construct.

Linkers of the disclosure (L³) may have from about 10 to about 500 atoms in a linker, such as from about 10 to about 400 atoms, such as about 10 to about 300 atoms in a linker. In certain embodiments, linkers of the disclosure have from about 30 to about 400 atoms, such as from about 30 to about 300 atoms in the linker.

The linkers may link a compound or salt of any one of Formulas (IA), (IB), (IC), (VIIA), (VIIB), or (VIIC) to the antibody construct by a covalent linkages between the linker and the antibody construct and compound. As used herein, the expression “linker” is intended to include (i) unconjugated forms of the linker that include a functional group capable of covalently linking the linker to an imidazoquinoline compound(s) and a functional group capable of covalently linking the linker to an antibody construct; (ii) partially conjugated forms of the linker that include a functional group capable of covalently linking the linker to an antibody construct and that is covalently linked to a compound(s) or salt(s) of any one of Formulas (IA), (IB), (IC), (VIIA), (VIIB), or (VIIC), or vice versa; and (iii) fully conjugated forms of the linker that is covalently linked to both a compound(s) or salt(s) of any one of Formulas (IA), (IB), (IC), (VIIA), (VIIB), or (VIIC) and an antibody construct. One embodiment pertains to a conjugate formed by contacting an antibody construct that binds a cell surface receptor or tumor associated antigen expressed on a tumor cell with a linker-compound described herein under conditions in which the linker-compound covalently links to the antibody construct. One embodiment pertains to a method of making a conjugate formed by contacting a linker-compound described herein under conditions in which the linker-compound covalently links to the antibody construct. One embodiment pertains to a method of stimulating immune activity in a cell that expresses CD40, comprising contacting the cell with a conjugate described herein that is capable of binding the cell, under conditions in which the conjugate binds the cell.

Polyvalent linkers that may be used to link many imidazoquinoline compounds to an antibody construct are described. For example, Fleximer® linker technology has the potential to enable high-DAR conjugates with good physicochemical properties. As shown below, the Fleximer® linker technology is based on incorporating drug molecules into a solubilizing poly-acetal backbone via a sequence of ester bonds. The methodology renders highly-loaded conjugates (DAR up to 20) whilst maintaining good physicochemical properties. This methodology could be utilized with a imidazoquinoline compound.

To utilize the Fleximer® linker technology depicted in the scheme above, an aliphatic alcohol can be present or introduced into a compound of any one of Formulas (IA), (IB), (IC), (VIIA), (VIIB), or (VIIC). The alcohol moiety is then conjugated to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates described herein are described below.

Cleavable linkers can be cleavable in vitro and in vivo. Cleavable linkers can include chemically or enzymatically unstable or degradable linkages. Cleavable linkers can rely on processes inside the cell to liberate an imidazoquinoline compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate imidazoquinoline compound release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the compound once the antibody construct conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Antibody construct conjugates including exemplary hydrazone-containing linkers can include, for example, the following structures:

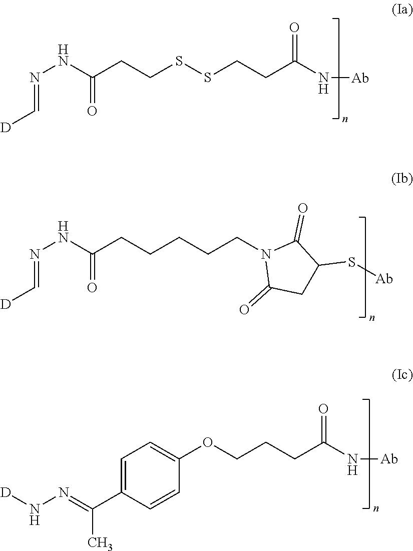

wherein D is a compound or salt of any one of Formulas (IA), (IB), (IC), (VIIA), (VIIB), or (VIIC) and Ab is an antibody construct, respectively, and n represents the number of compound-bound linkers (LP) bound to the antibody construct. In certain linkers, such as linker (Ia), the linker can comprise two cleavable groups, a disulfide and a hydrazone moiety. For such linkers, effective release of the unmodified free compound can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the imidazoquinoline compound upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the compound in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Antibody construct conjugates including exemplary disulfide-containing linkers can include the following structures:

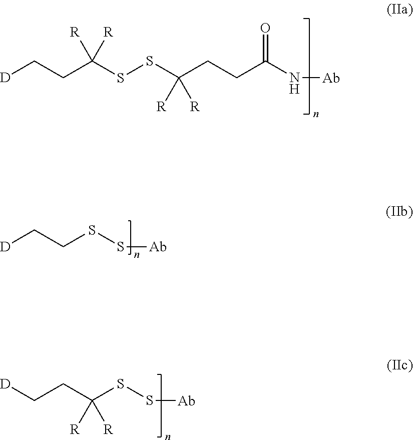

wherein D is a compound or salt of any one of Formulas (IA), (IB), (IC), (VIIA), (VIIB), or (VIIC), and Ab is an antibody construct, respectively, n represents the number of compounds bound to linkers (L³) bound to the antibody construct and R is independently selected at each occurrence from hydrogen or alkyl, for example. Increasing steric hindrance adjacent to the disulfide bond can increase the stability of the linker. Structures such as (IIa) and (IIc) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a compound from an antibody construct can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, cathepsin S, β-glucuronidase, or β-galactosidase.

The cleavable peptide can be selected from tetrapeptides such as Gly-Phe-Leu-Gly, Ala-Leu-Ala-Leu or dipeptides such as Val-Cit, Val-Ala, and Phe-Lys. Dipeptides can have lower hydrophobicity compared to longer peptides.

A variety of dipeptide-based cleavable linkers can be used in the antibody construct conjugates described herein.

Enzymatically cleavable linkers can include a self-immolative spacer to spatially separate the imidazoquinoline compound from the site of enzymatic cleavage. The direct attachment of a compound to a peptide linker can result in proteolytic release of the compound or of an amino acid adduct of the compound, thereby impairing its activity. The use of a self-immolative spacer can allow for the elimination of the fully active, chemically unmodified compound upon amide bond hydrolysis.

One self-immolative spacer can be a bifunctional para-aminobenzyl alcohol group, which can link to the peptide through the amino group, forming an amide bond, while amine containing compounds can be attached through carbamate functionalities to the benzylic hydroxyl group of the linker (to give a p-amidobenzylcarbamate, PABC). The resulting pro-imidazoquinoline compound can be activated upon protease-mediated cleavage, leading to a 1,6-elimination reaction releasing the unmodified compound, carbon dioxide, and remnants of the linker. The following scheme depicts the fragmentation of p-amidobenzyl carbamate and release of the compound:

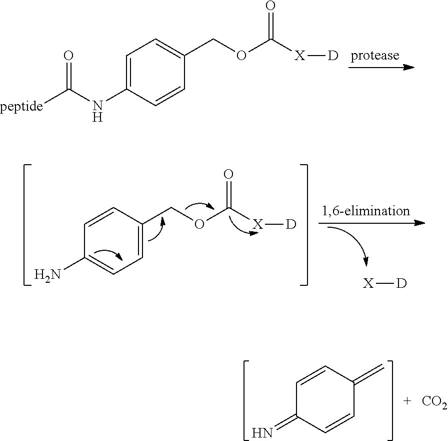

wherein X-D represents the unmodified compound.
Heterocyclic variants of this self-immolative group have also been described.

The enzymatically cleavable linker can be a β-glucuronic acid-based linker. Facile release of the compound can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of an antibody construct imidazoquinoline compound conjugate to undergo aggregation due to the hydrophilic nature of β-glucuronides. In certain embodiments, β-glucuronic acid-based linkers can link an antibody construct to a hydrophobic imidazoquinoline compound. The following scheme depicts the release of a imidazoquinoline compound (D) from an antibody construct imidazoquinoline compound conjugate containing a β-glucuronic acid-based linker:

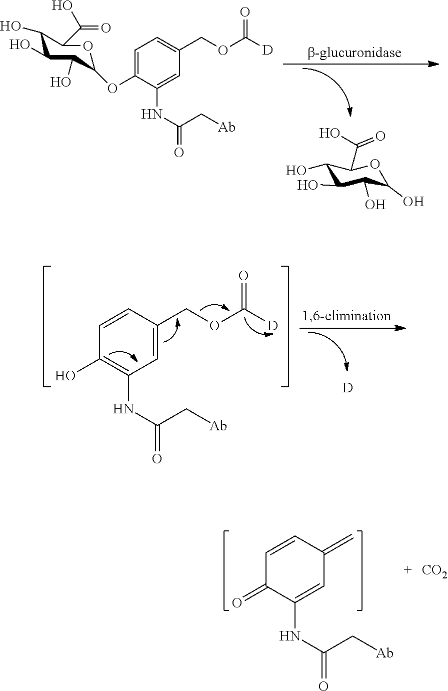

wherein Ab indicates the antibody construct.

A variety of cleavable β-glucuronic acid-based linkers useful for linking drugs such as auristatins, camptothecin and doxorubicin analogues, CBI minor-groove binders, and psymberin to antibodies have been described. These β-glucuronic acid-based linkers may be used in the conjugates described herein. In certain embodiments, the enzymatically cleavable linker is a β-galactoside-based linker. β-Galactoside is present abundantly within lysosomes, while the enzyme activity outside cells is low.

Additionally, imidazoquinoline compounds containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on a imidazoquinoline compound, wherein such ester groups can hydrolyze under physiological conditions to release the imidazoquinoline compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide moiety, for example, a linker comprising structural formula (IIIa), (IIIb), (IIIc), or (IIId):

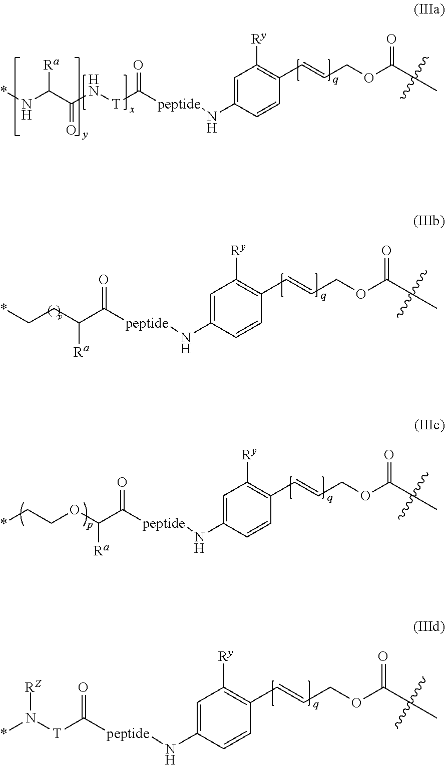

or a salt thereof, wherein. “peptide” represents a peptide (illustrated in N→C orientation, wherein peptide includes the amino and carboxy “termini”) that is cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or a sugar moiety; G² is SO₃H, CO₂H, or a PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to a compound or salt of any one of Formulas (IA), (IB), (IC), (VIIA), (VIIB), or (VIIC); and * represents the point of attachment to the remainder of the linker.

In certain embodiments, the peptide can be selected from natural amino acids, unnatural amino acids or combinations thereof. In certain embodiments, the peptide can be selected from a tripeptide or a dipeptide. In particular embodiments, the dipeptide can comprise L-amino acids and be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Embodiments of linkers according to structural formula (IIIa) are illustrated below (as illustrated, the linkers include a reactive group suitable for covalently linking the linker to an antibody construct):

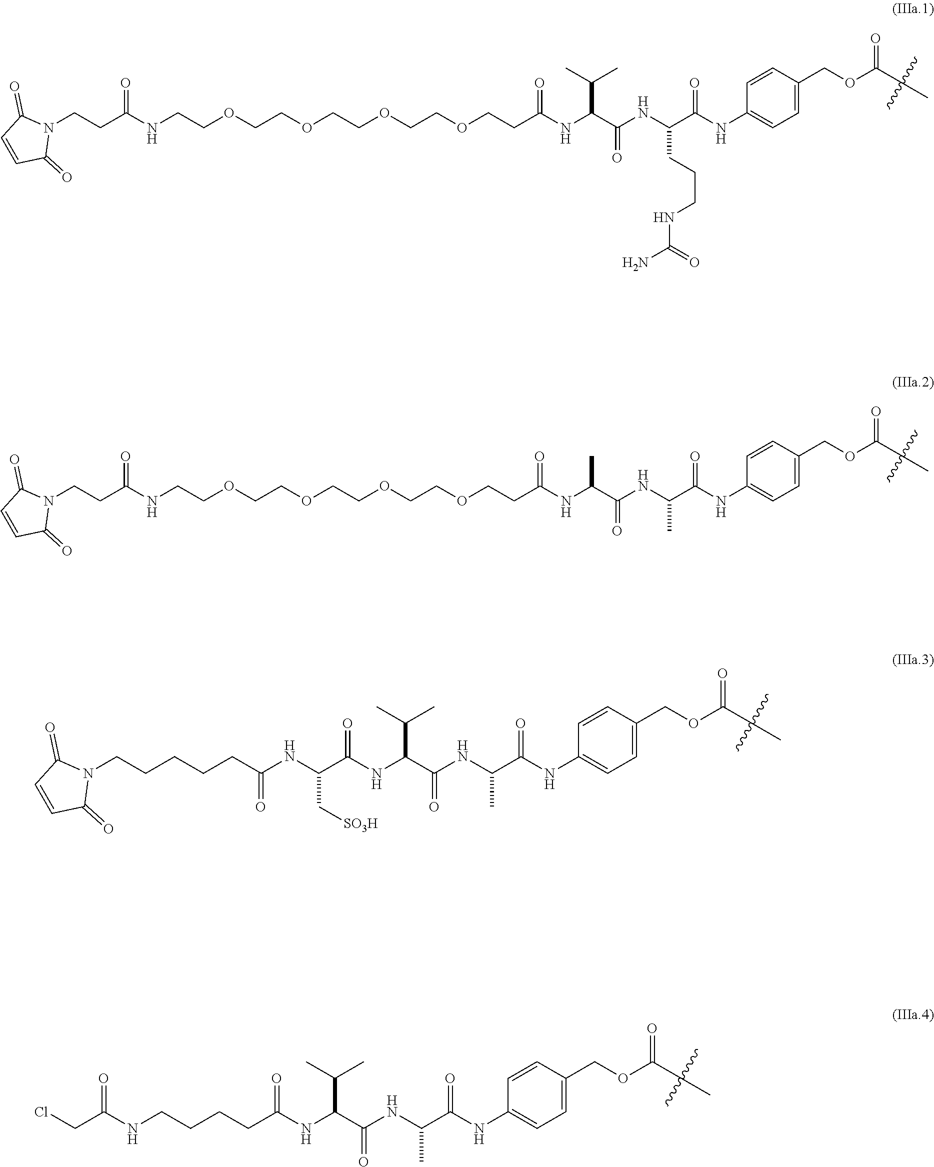 [SMILES: *C(=O)OCc1ccc(NC(=O)[C@H](CCCNC(N)=O)NC(=O)[C@@H](NC(=O)CCOCCOCCOCCOCCNC(=O)CCN2C(=O)C=CC2=O)C(C)C)cc1 ; *C(=O)OCc1ccc(NC(=O)[C@H](C)NC(=O)[C@H](C)NC(=O)CCOCCOCCOCCOCCNC(=O)CCN2C(=O)C=CC2=O)cc1 ; *C(=O)OCc1ccc(NC(=O)[C@H](C)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCNC(=O)CCl)C(C)C)cc1]

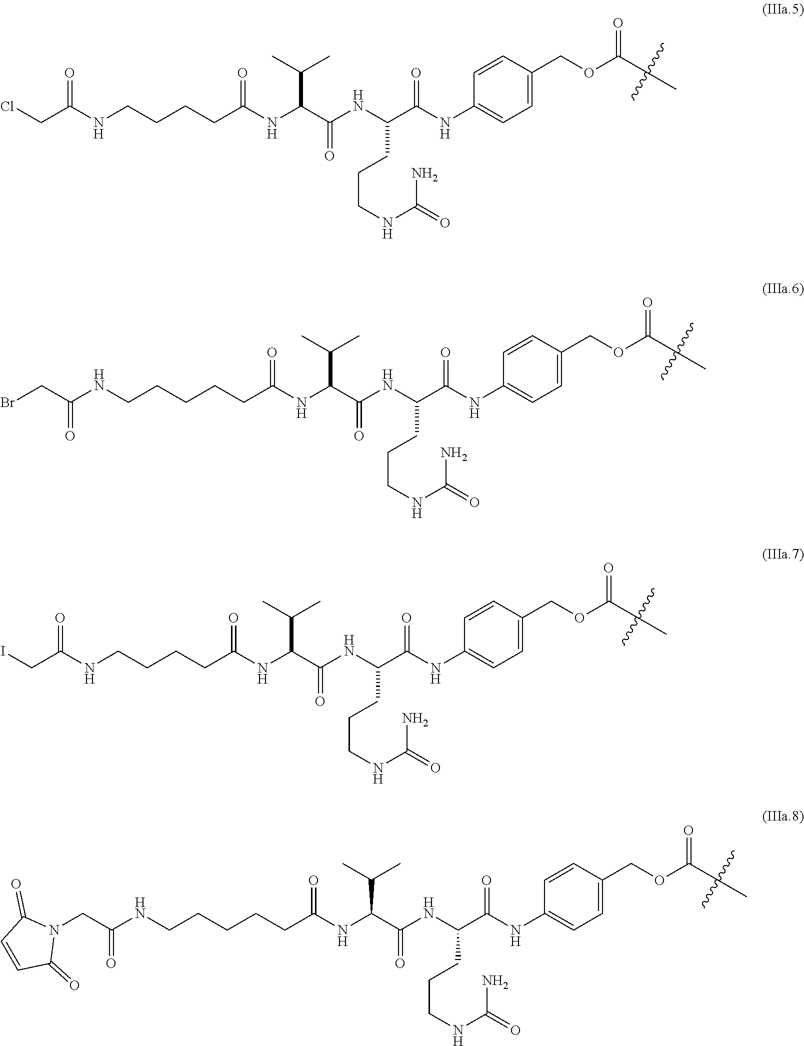 [SMILES: *C(=O)OCc1ccc(NC(=O)[C@H](CCCNC(N)=O)NC(=O)[C@@H](NC(=O)CCCCNC(=O)CCl)C(C)C)cc1 ; *C(=O)OCc1ccc(NC(=O)[C@H](CCCNC(N)=O)NC(=O)[C@@H](NC(=O)CCCCCNC(=O)CBr)C(C)C)cc1 ; *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

wherein [symbol] indicates an attachment site of a linker (L³) to a compound or salt of any one of Formulas (IA), (IB), (IC), (VIIA), (VIIB), or (VIIC).

Embodiments of linkers according to structural formula (IIb), (IIc), or (IId) that can be included in the conjugates described herein can include the linkers illustrated below (as illustrated, the linkers can include a reactive group suitable for covalently linking the linker to an antibody construct):

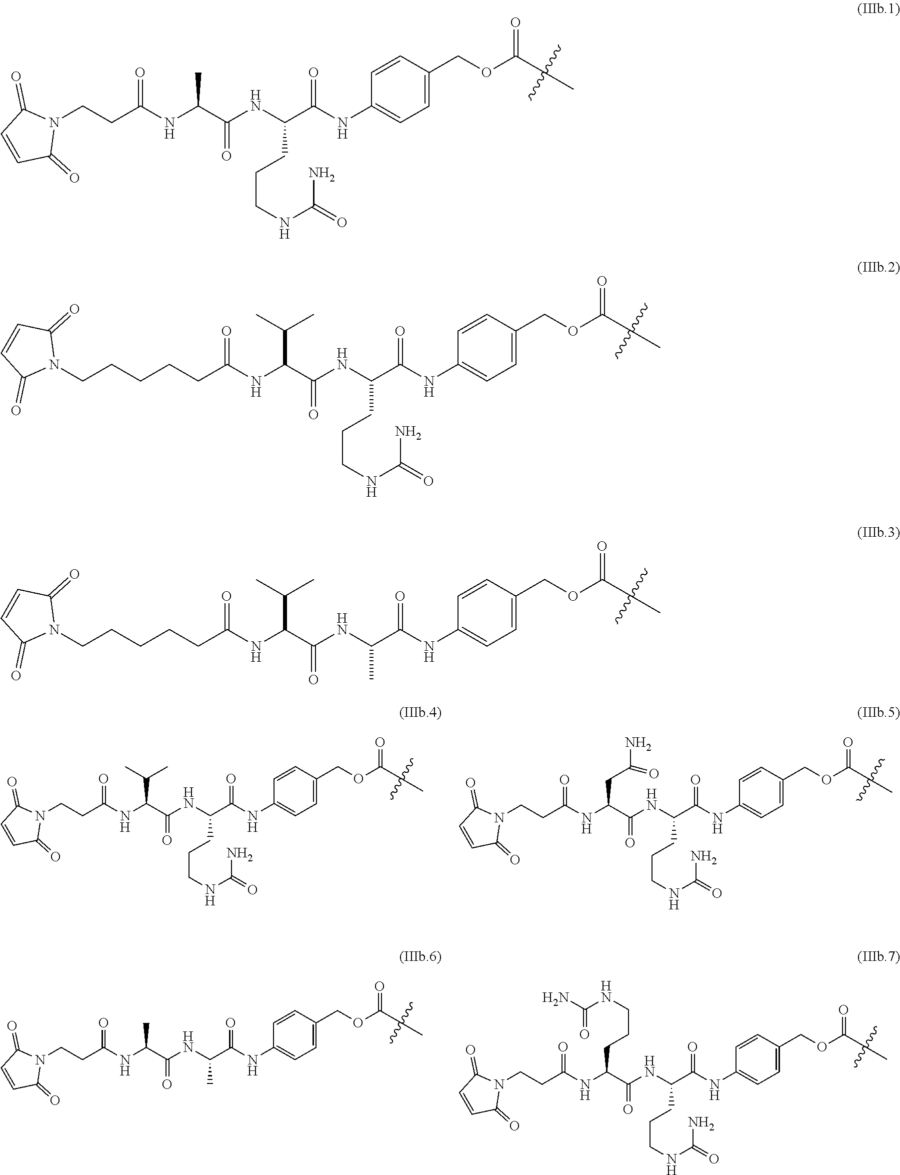

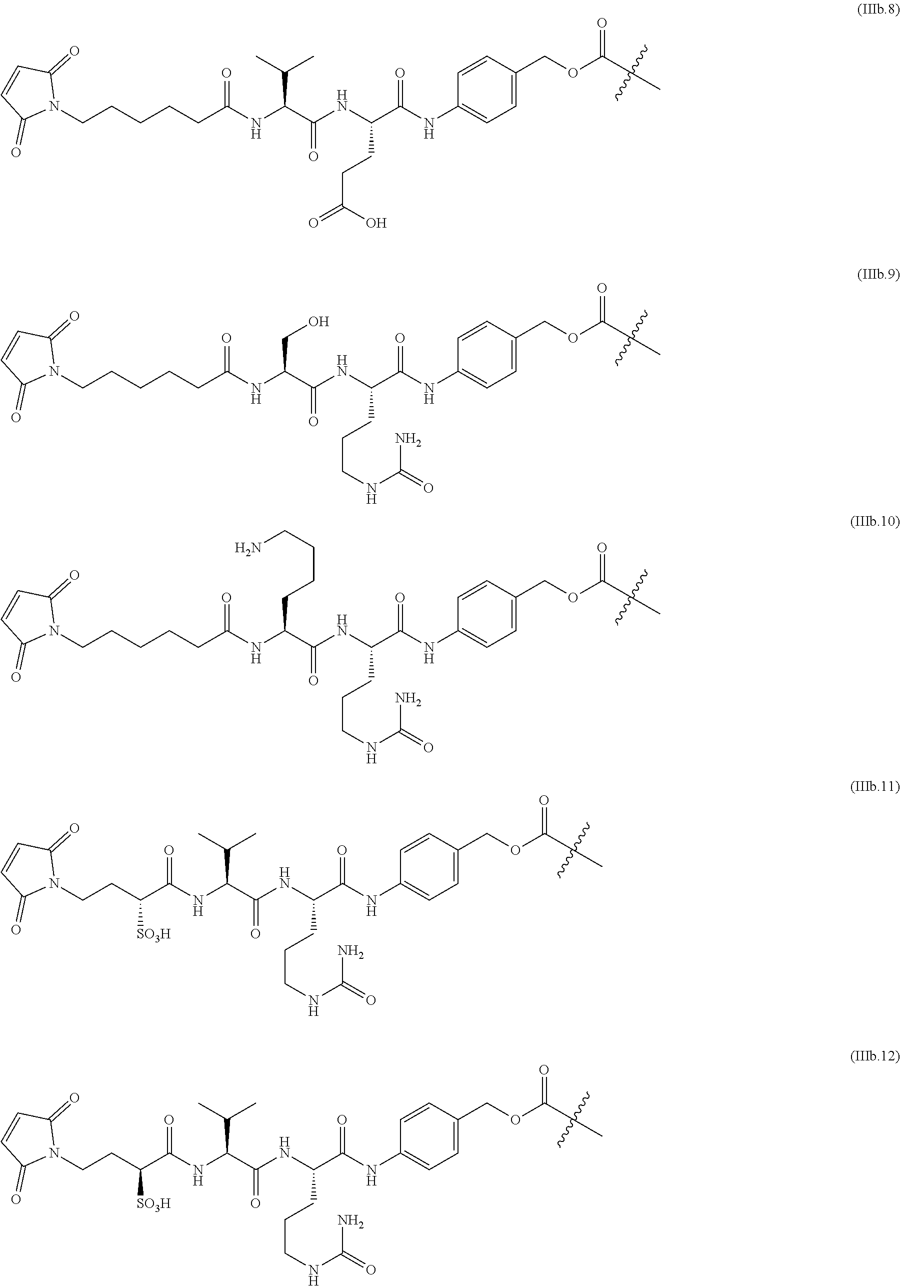 [SMILES: *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1]

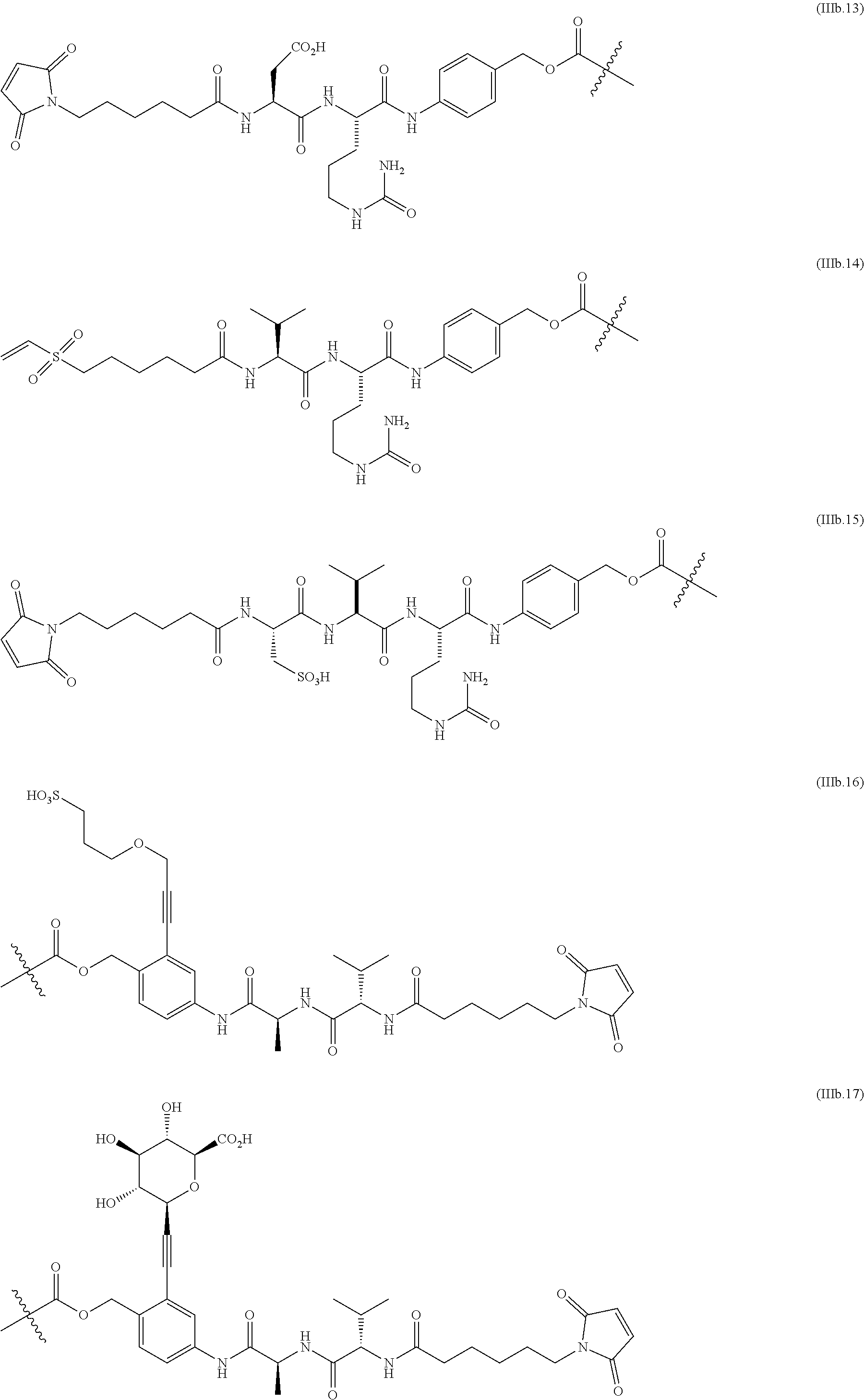

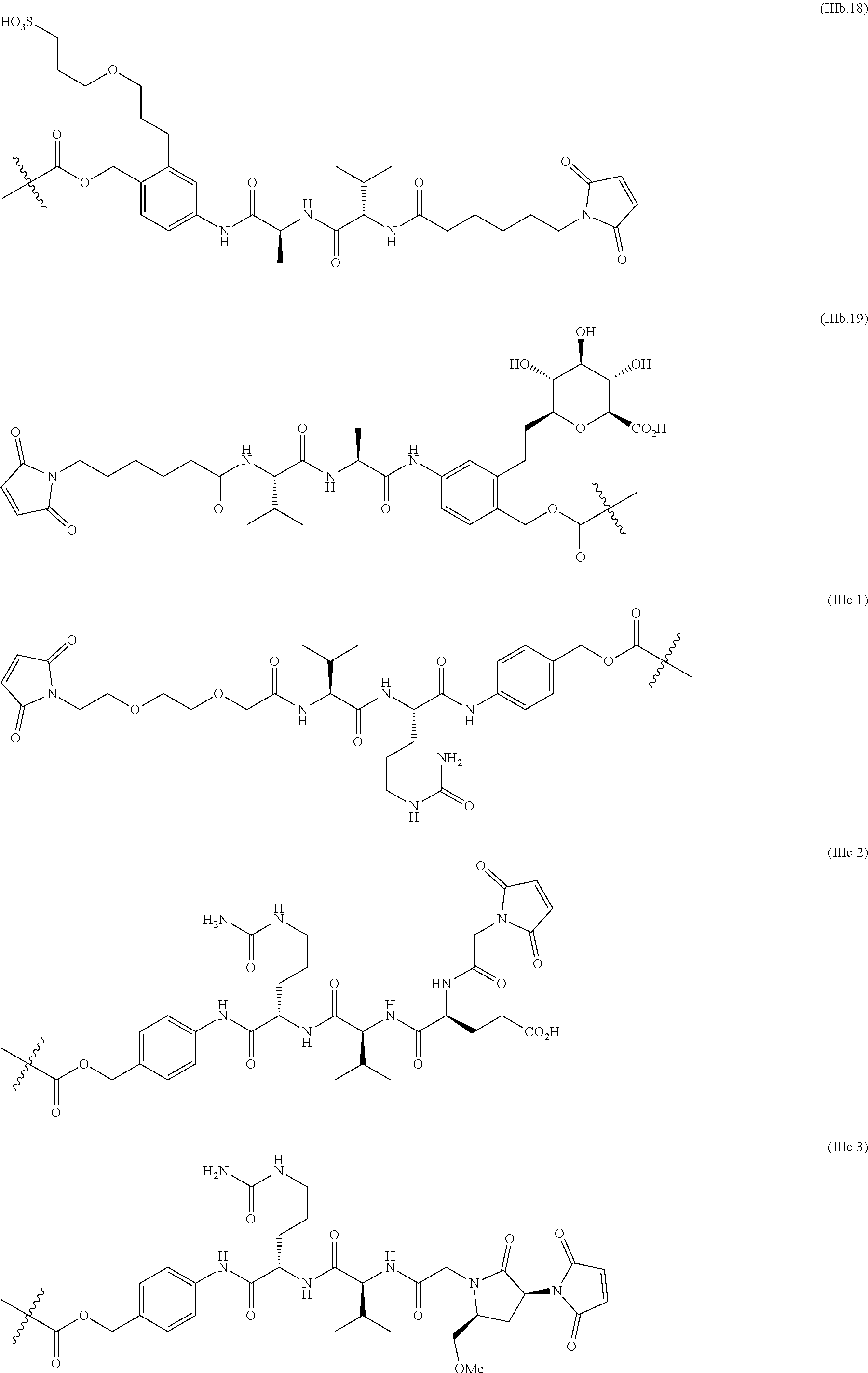

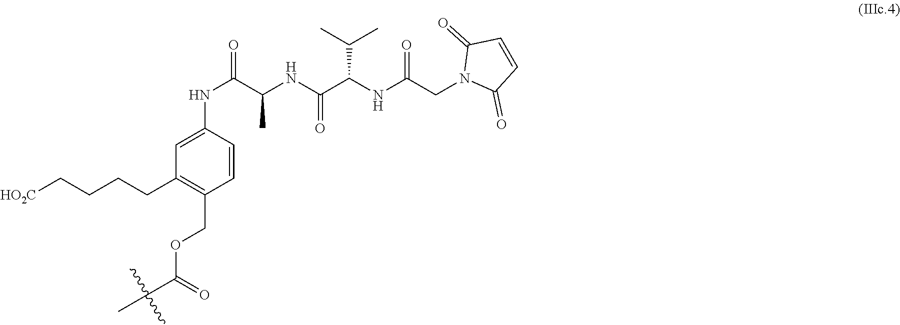 [SMILES: *C(=O)OCc1ccc(NC(=O)[C@H](C)NC(=O)[C@@H](NC(=O)CN2C(=O)C=CC2=O)C(C)C)cc1CCCCC(=O)O]

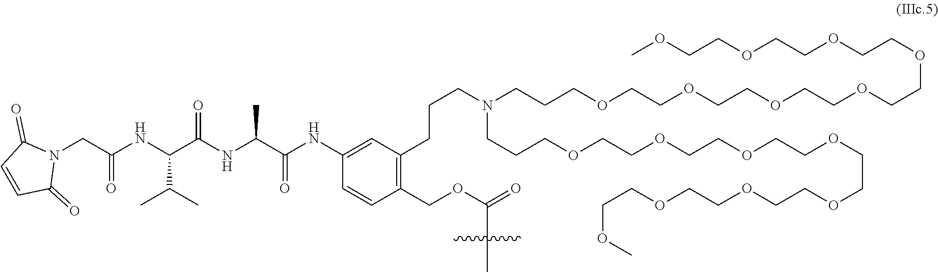 [SMILES: *C(=O)OCc1ccc(NC(=O)[C@H](C)NC(=O)[C@@H](NC(=O)CN2C(=O)C=CC2=O)C(C)C)cc1CCCN(CCCOCCOCCOCCOCCOCCOCCOCCOC)CCCOCCOCCOCCOCCOCCOCCOCCOC]

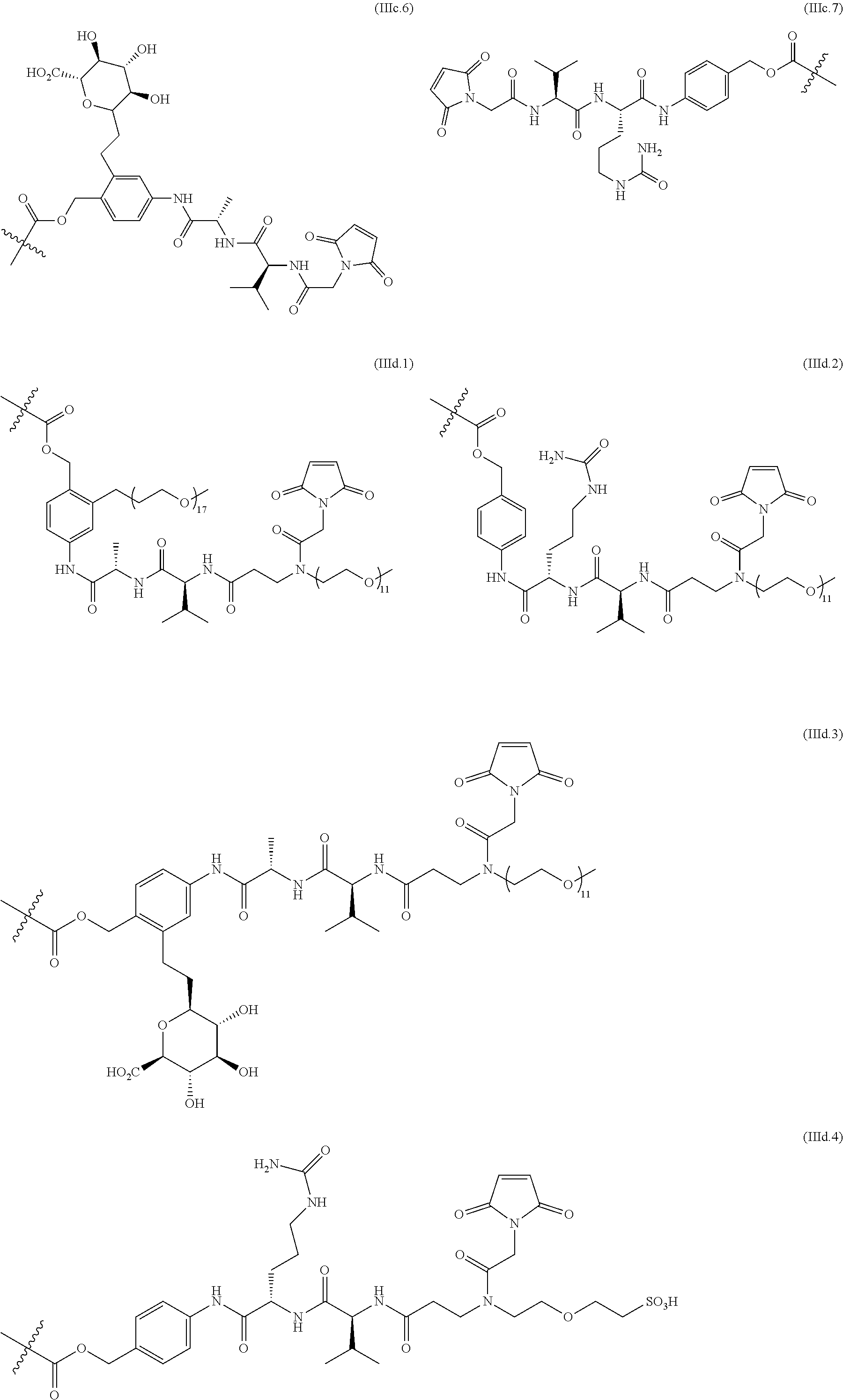

wherein [symbol] indicates an attachment site to a compound or salt of any one of Formulas (IA), (IB), (IC), (VIIA), (VIIB), or (VIIC).

The linker can contain an enzymatically cleavable sugar moiety, for example, a linker comprising structural formula (IVa), (IVb), (IVc), (IVd), or (IVe):

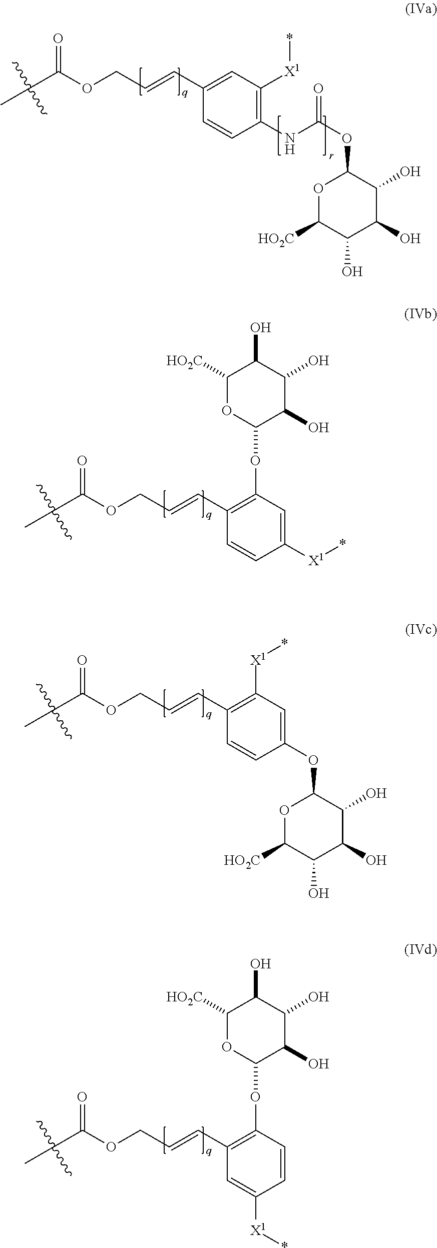

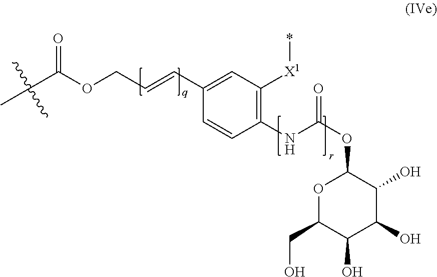

or a salt thereof, wherein. q is 0 or 1; r is 0 or 1; X¹ is CH₂, O or NH; [symbol] represents the point of attachment of the linker (L³) to the compound or salt of any one of Formulas (IA), (IB), (IC), (VIIA), (VIIB), or (VIIC); and * represents the point of attachment to the remainder of the linker.

Embodiments of linkers according to structural formula (IVa) that may be included in the antibody construct conjugates described herein can include the linkers illustrated below (as illustrated, the linkers include a group suitable for covalently linking the linker to an antibody construct):

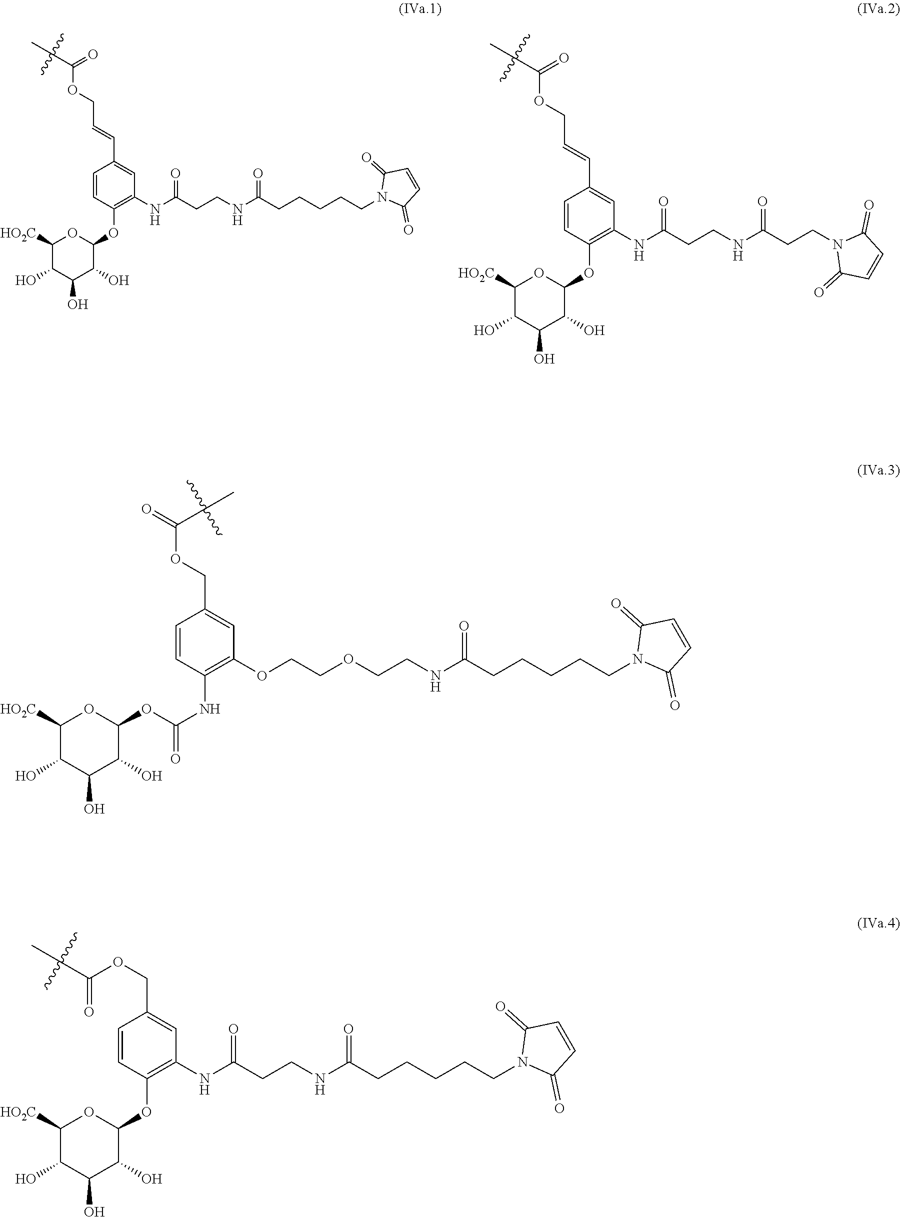

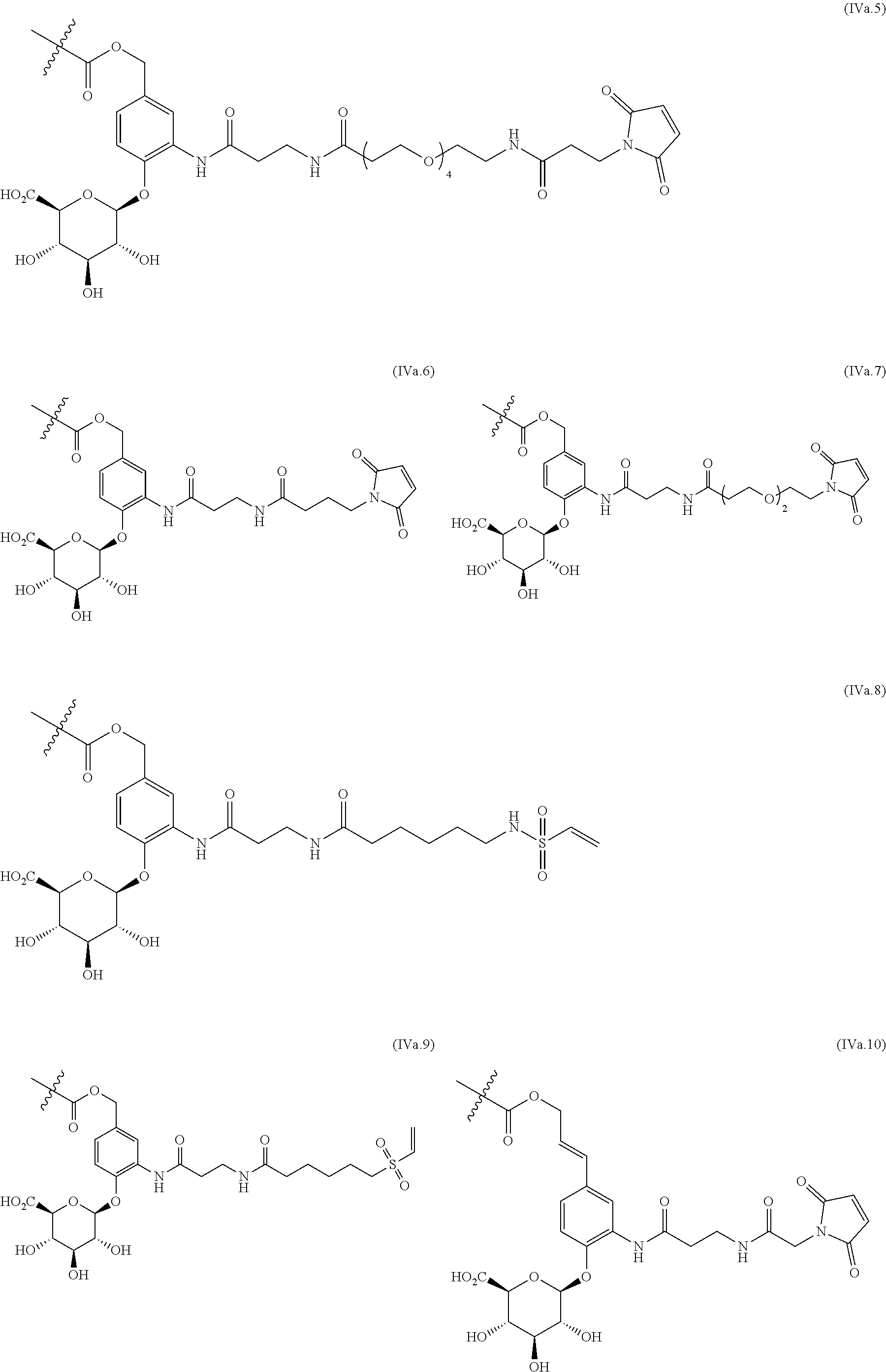

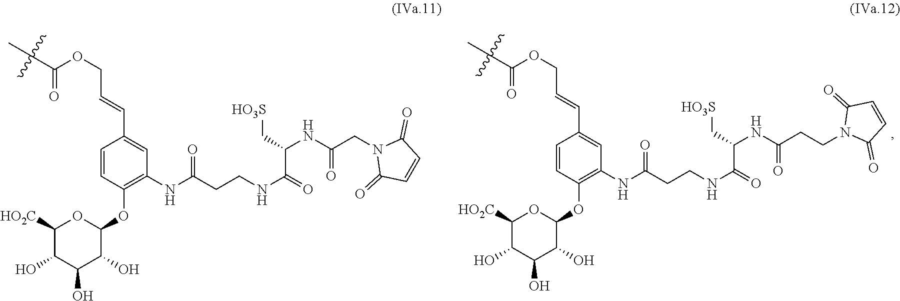

wherein [symbol] represents the point of attachment of the linker (L³) to the compound or salt of any one of Formulas (IA), (IB), (IC), (VIIA), (VIIB), or (VIIC).

Embodiments of linkers according to structural formula (IVb) that may be included in the conjugates described herein include the linkers illustrated below (as illustrated, the linkers include a group suitable for covalently linking the linker to an antibody construct):

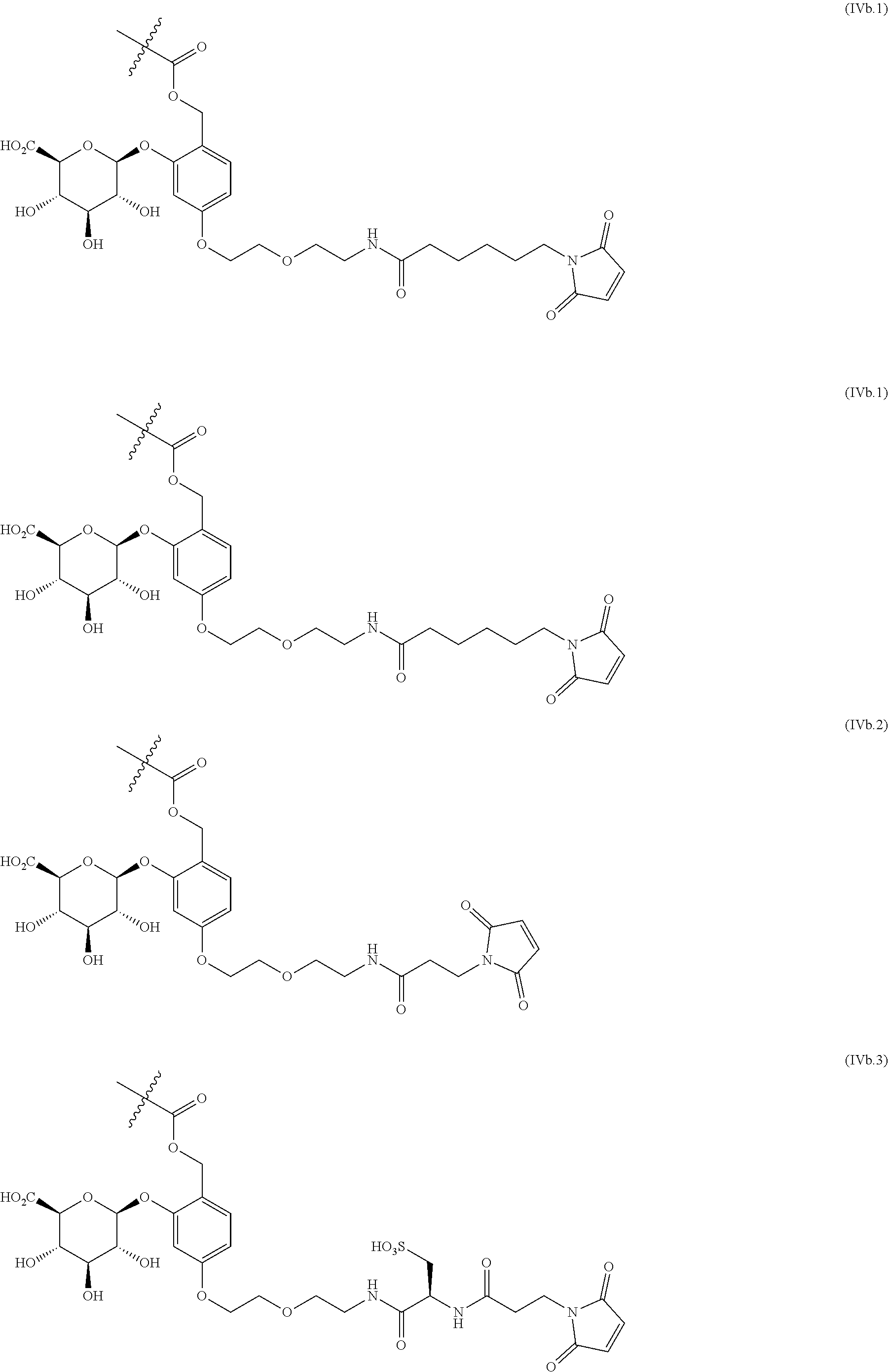

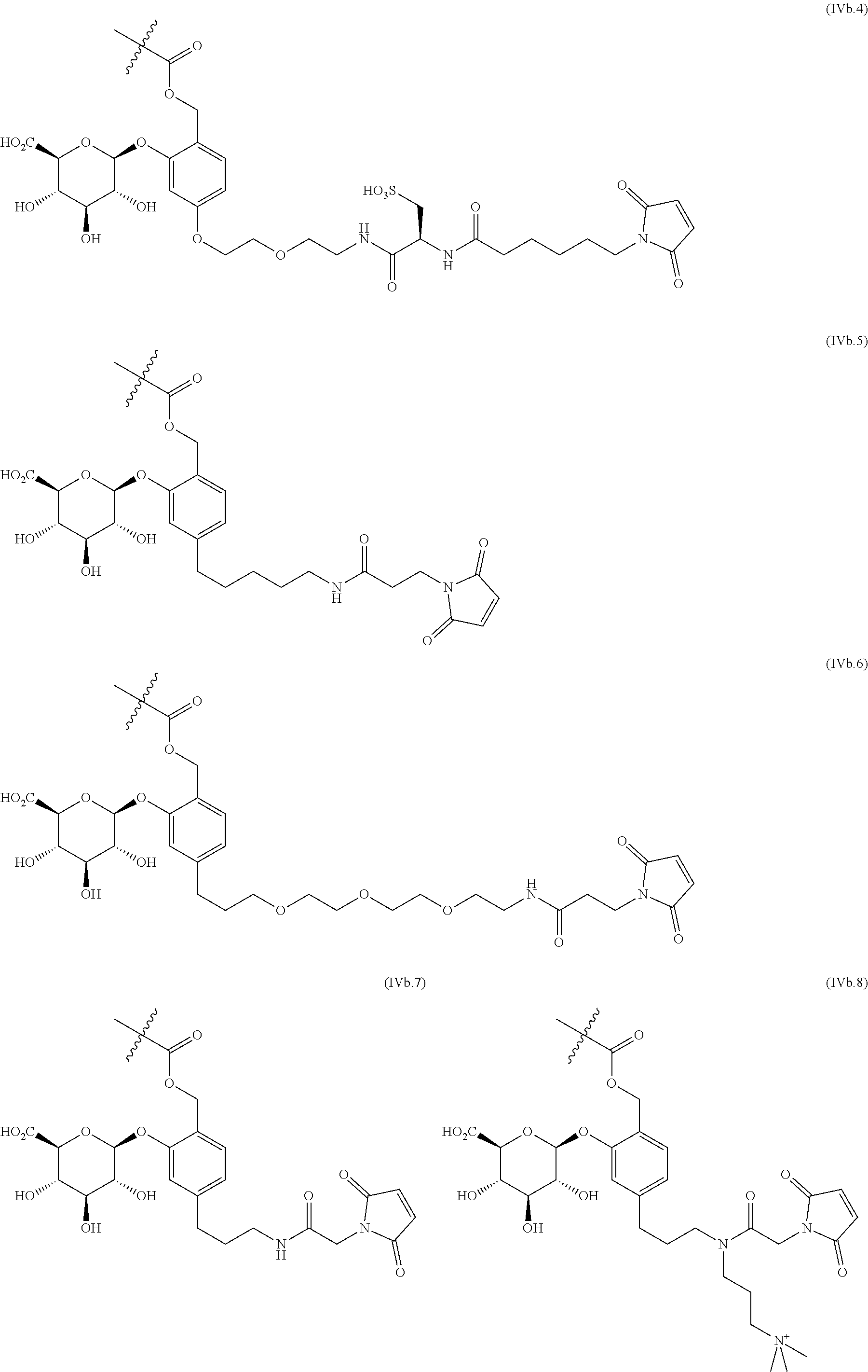

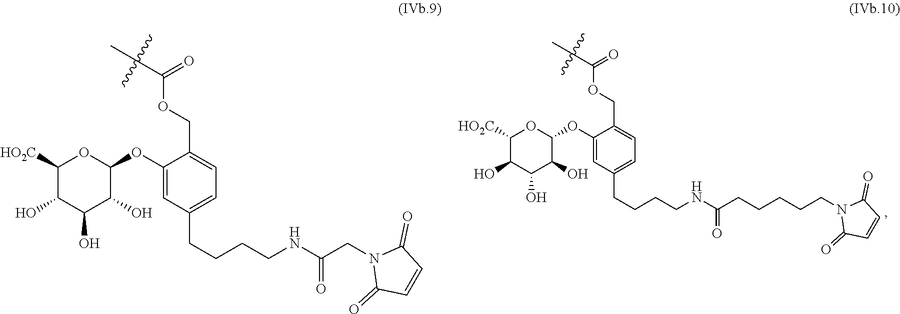

wherein [symbol] represents the point of attachment of the linker (L³) to the compound or salt of any one of Formulas (IA), (IB), (IC), (VIIA), (VIIB), or (VIIC).

Embodiments of linkers according to structural formula (IVc) that may be included in the conjugates described herein include the linkers illustrated below (as illustrated, the linkers include a group suitable for covalently linking the linker to an antibody construct):

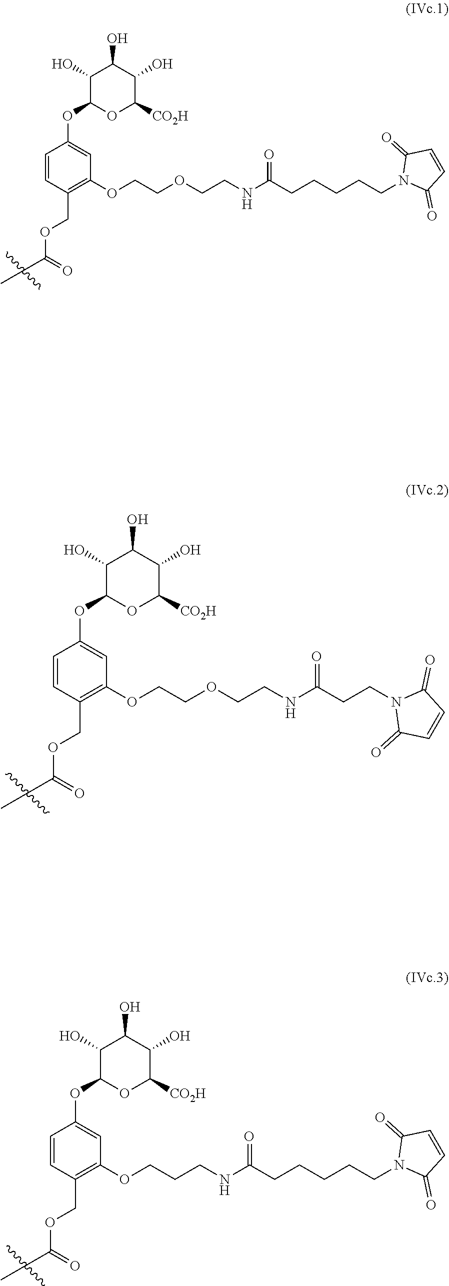

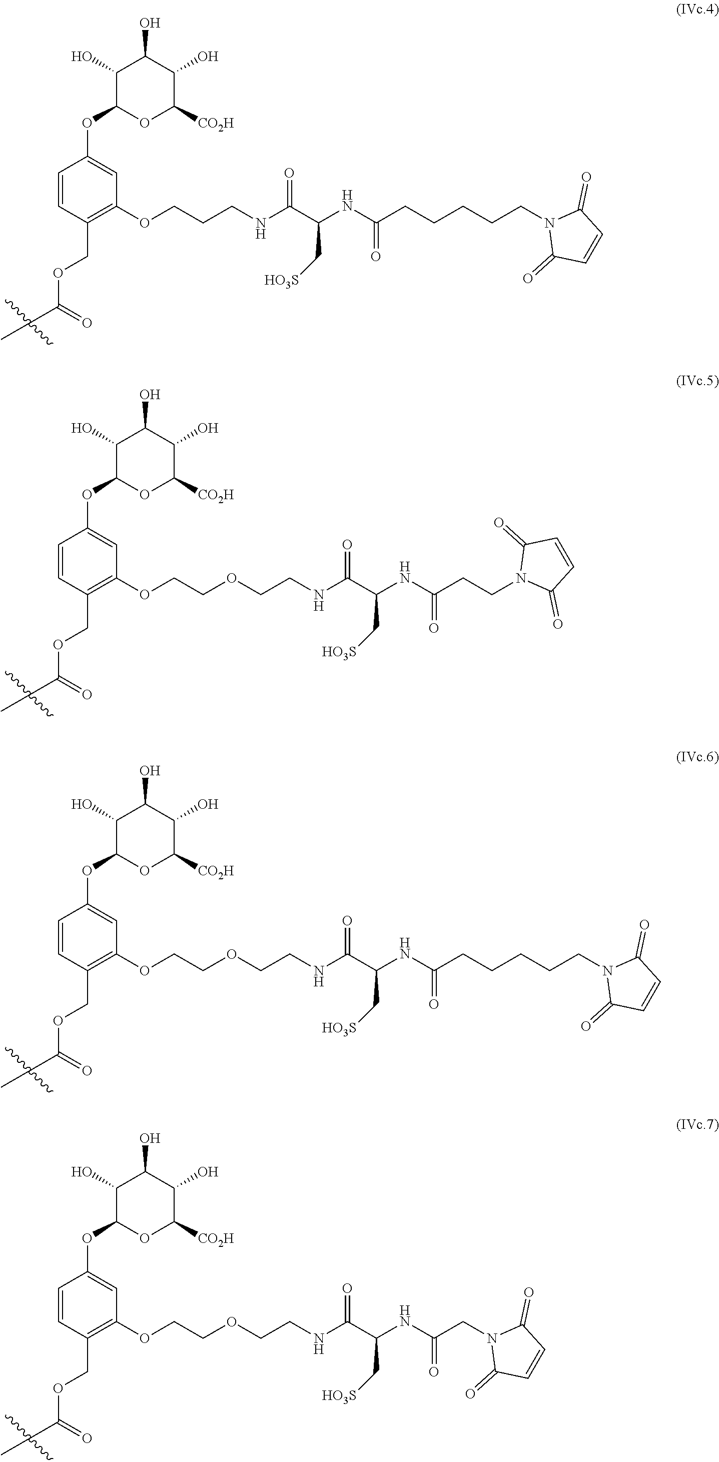

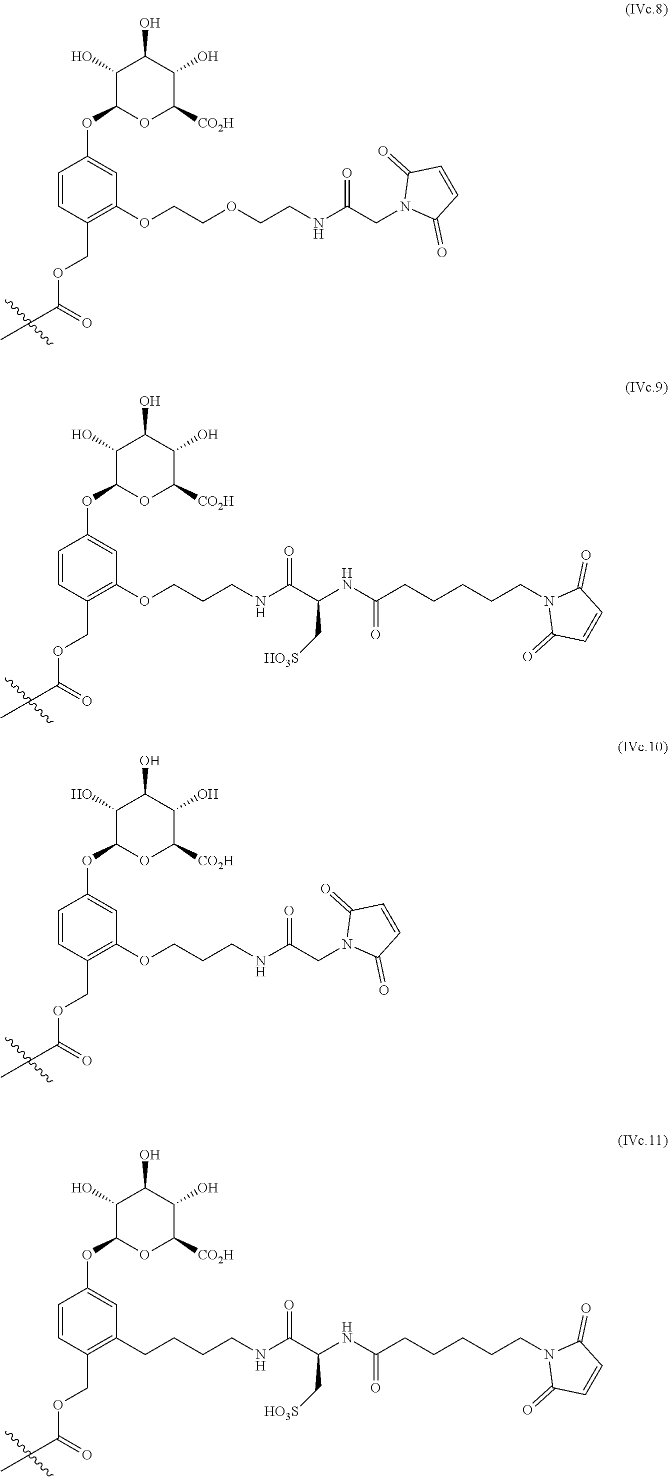

wherein [symbol] represents the point of attachment of the linker (L³) to the compound or salt of any one of Formulas (IA), (IB), (IC), (VIIA), (VIIB), or (VIIC).

Embodiments of linkers according to structural formula (IVd) that may be included in the conjugates described herein include the linkers illustrated below (as illustrated, the linkers include a group suitable for covalently linking the linker to an antibody construct):

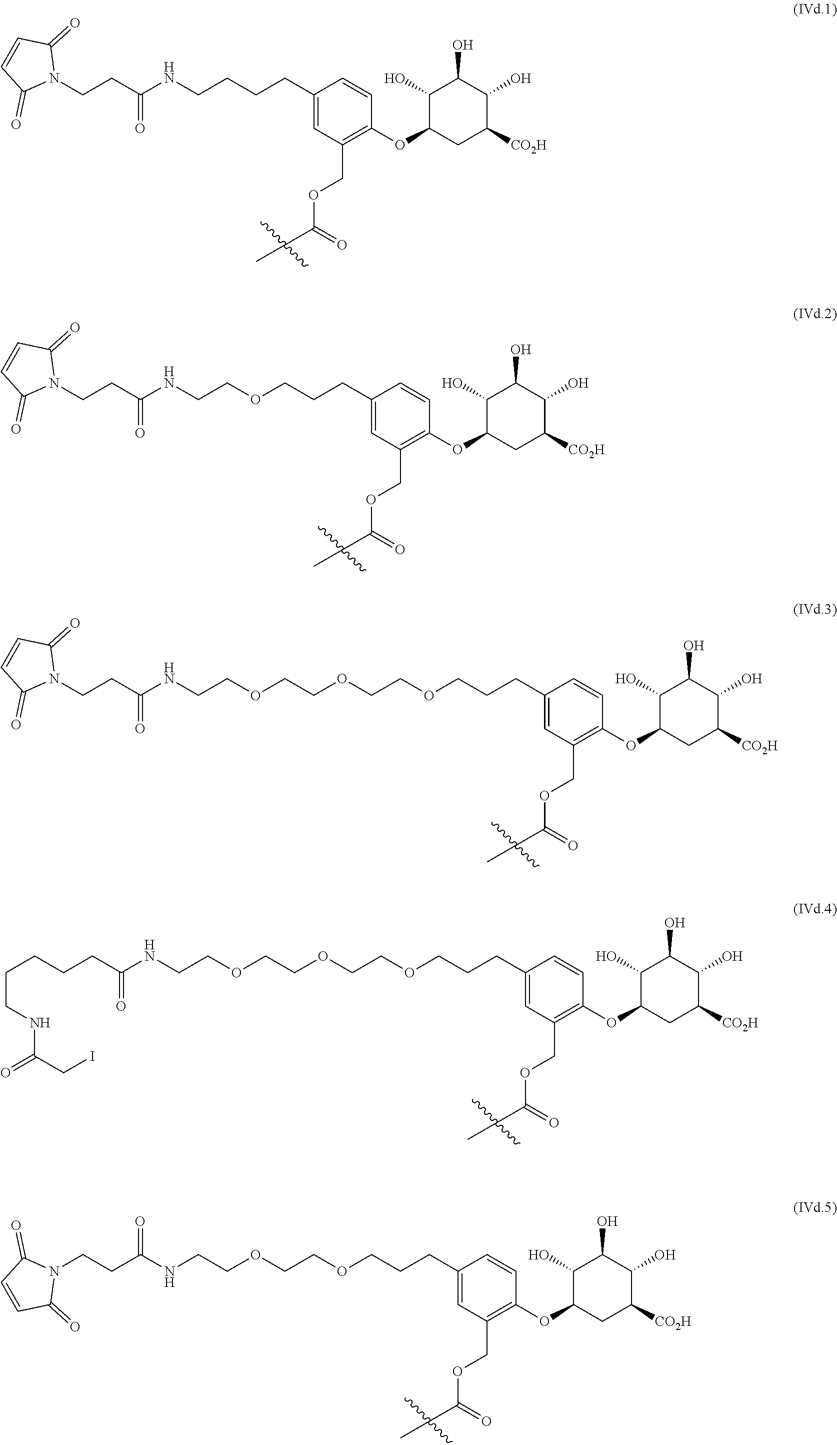

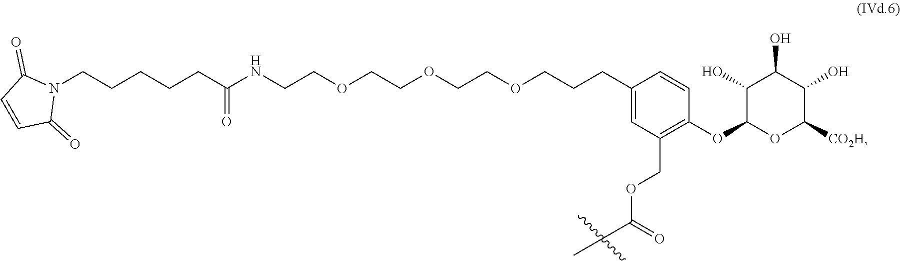

wherein [symbol] represents the point of attachment of the linker (L³) to the compound or salt of any one of Formulas (IA), (IB), (IC), (VIIA), (VIIB), or (VIIC).

Embodiments of linkers according to structural formula (IVe) that may be included in the conjugates described herein include the linkers illustrated below (as illustrated, the linkers include a group suitable for covalently linking the linker to an antibody construct):

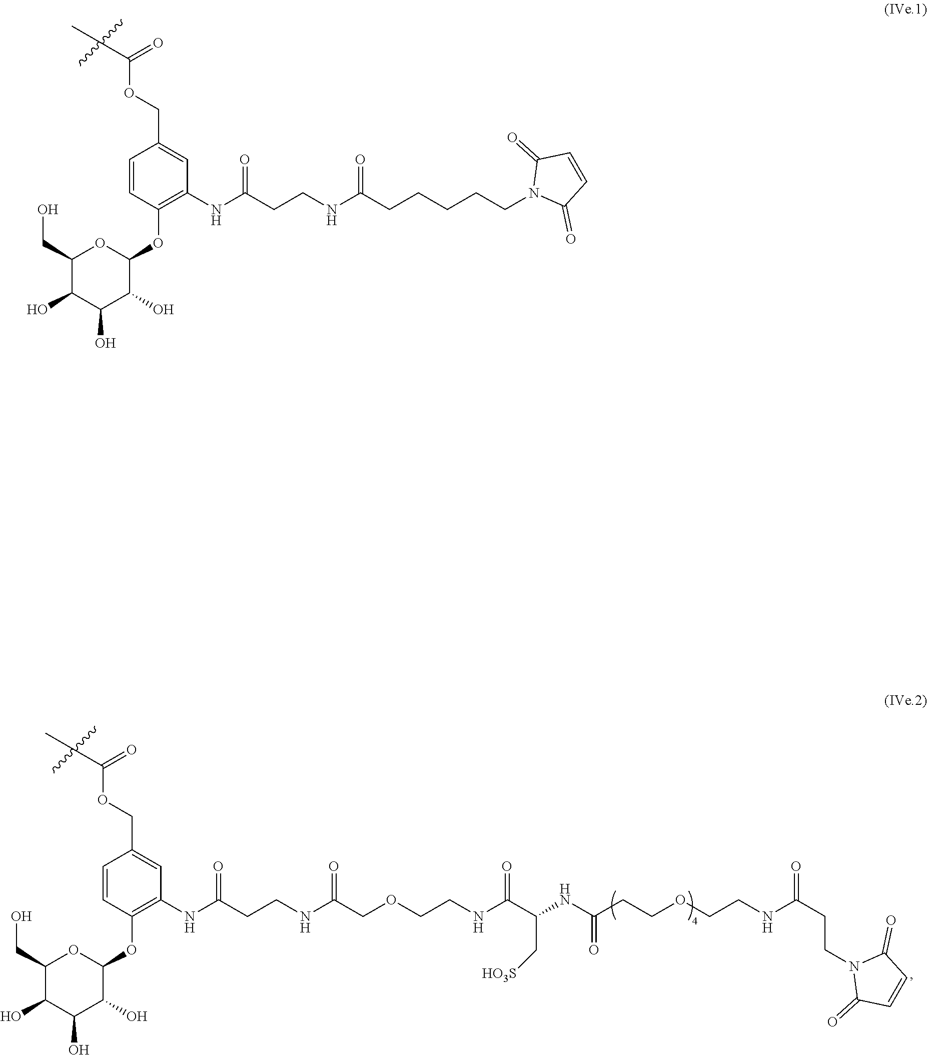

wherein [symbol] represents the point of attachment of the linker (L³) to the compound or salt of any one of Formulas (IA), (IB), (IC), (VIIA), (VIIB), or (VIIC).

Although cleavable linkers can provide certain advantages, the linkers comprising the conjugate described herein need not be cleavable. For non-cleavable linkers, the compound release may not depend on the differential properties between the plasma and some cytoplasmic compartments. The release of the compound can occur after internalization of the antibody construct compound conjugate via antigen-mediated endocytosis and delivery to lysosomal compartment, where the antibody construct can be degraded to the level of amino acids through intracellular proteolytic degradation. This process can release a compound derivative, which is formed by the compound, the linker, and the amino acid residue or residues to which the linker was covalently attached. The compound derivative from antibody construct conjugates with non-cleavable linkers can be more hydrophilic and less membrane permeable, which can lead to less bystander effects and less nonspecific toxicities compared to antibody construct conjugates with a cleavable linker. Antibody construct conjugates with non-cleavable linkers can have greater stability in circulation than antibody construct conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

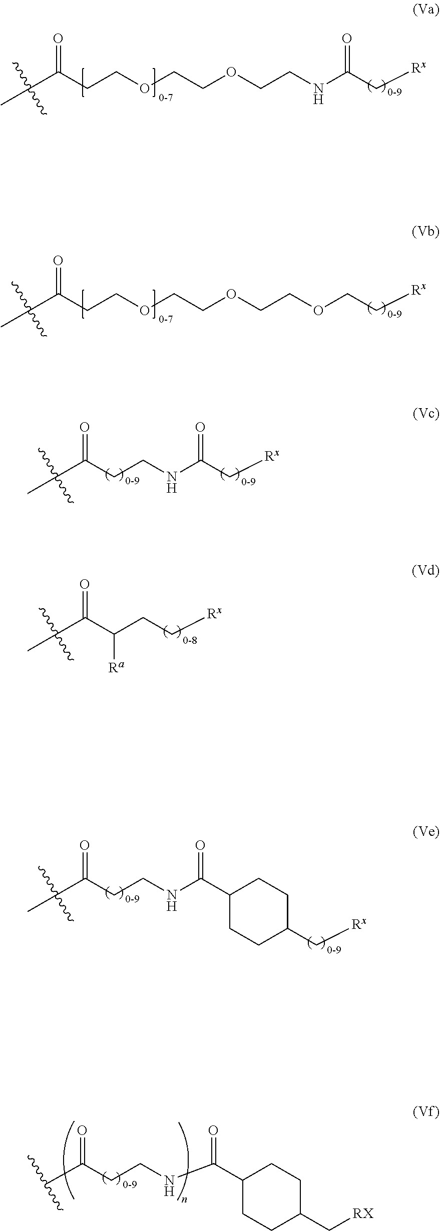

or salts thereof, wherein: R^{a} is selected from hydrogen, alkyl, sulfonate and methyl sulfonate; R^{x} is a reactive moiety including a functional group capable of covalently linking the linker to an antibody construct; and [symbol] represents the point of attachment of the linker (L³) to the compound or salt of any one of Formulas (IA), (IB), (IC), (VIIA), (VIIB), or (VIIC).

Embodiments of linkers according to structural formula (Va)-(Vf) that may be included in the conjugates described herein include the linkers illustrated below (as illustrated, the linkers include a group suitable for covalently linking the linker to an antibody construct, and [symbol] represents the point of attachment of the linker (L³) to the compound or salt of any one of Formulas (IA), (IB), (IC), (VIIA), (VIIB), or (VIIC):

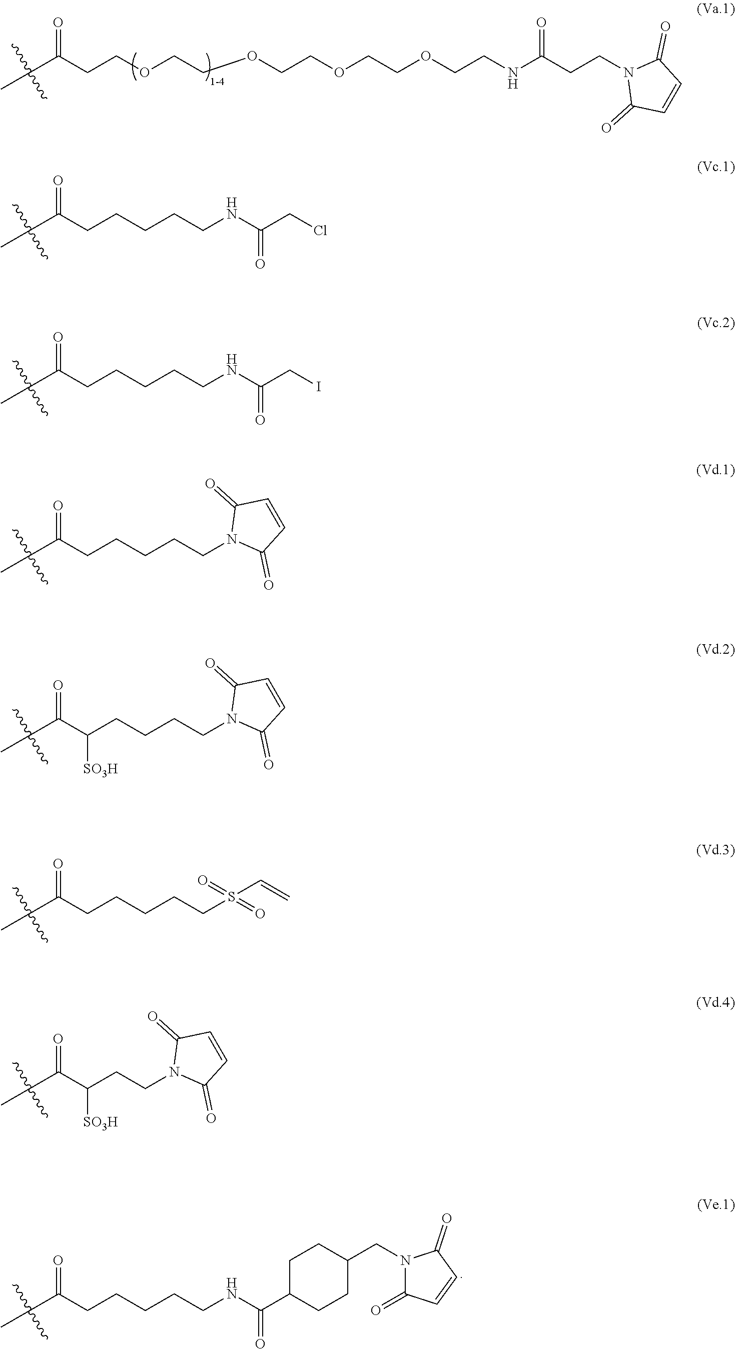

Attachment groups that are used to attach the linkers to an antibody construct can be electrophilic in nature and include, for example, maleimide groups, alkynes, alkynoates, allenes and allenoates, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

Maleimide groups are frequently used in the preparation of conjugates because of their specificity for reacting with thiol groups of, for example, cysteine groups of the antibody of a conjugate. The reaction between a thiol group of an antibody and a drug with a linker including a maleimide group proceeds according to the following scheme:

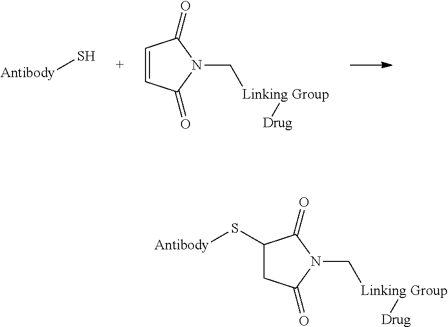

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability. A representative schematic is shown below:

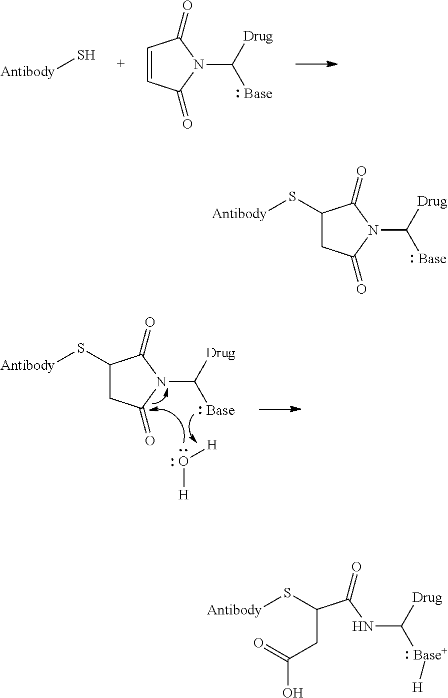

The hydrolysis reaction schematically represented above may occur at either carbonyl group of the succinimide group. Accordingly, two possible isomers may result, as shown below:

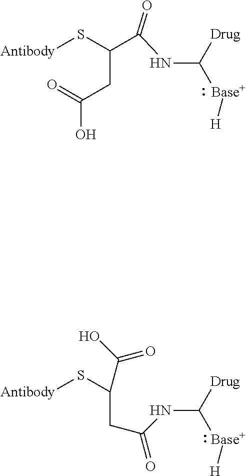

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate.

Bases suitable for inclusion in a linker described herein, e.g., any L³ described herein with a maleimide group prior to conjugating to an antibody construct, may facilitate hydrolysis of a nearby succinimide group formed after conjugation of the antibody construct to the linker. Bases may include, for example, amines (e.g., —N(R²⁶)(R²⁷), where R²⁶ and R²⁷ are independently selected from H and C₁₋₆ alkyl), nitrogen-containing heterocycles (e.g., a 3- to 12-membered heterocycle including one or more nitrogen atoms and optionally one or more double bonds), amidines, guanidines, and carbocycles or heterocycles substituted with one or more amine groups (e.g., a 3- to 12-membered aromatic or non-aromatic cycle optionally including a heteroatom such as a nitrogen atom and substituted with one or more amines of the type —N(R²⁶)(R²⁷), where R²⁶ and R²⁷ are independently selected from H or C₁₋₆ alkyl). A basic unit may be separated from a maleimide group by, for example, an alkylene chain of the form —(CH2)ₘ—, where m is an integer from 0 to 10. An alkylene chain may be optionally substituted with other functional groups as described herein.

A linker (L³) described herein with a maleimide group may include an electron withdrawing group such as, but not limited to, —C(O)R, ═O, —CN, —NO₂, —CX₃, —X, —COOR, —CONR₂, —COR, —COX, —SO₂R, —SO₂OR, —SO₂NHR, —SO₂NR₂, PO₃R₂, —P(O)(CH₃)NHR, —NO, —NR₃⁺, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups such as those described herein.

Examples of self-stabilizing linkers are provided in, e.g., U.S. Patent Publication Number 2013/0309256, the linkers of which are incorporated by reference herein. It will be understood that a self-stabilizing linker useful in conjunction with the compounds of the present disclosure may be equivalently described as unsubstituted maleimide-including linkers, thio-substituted succinimide-including linkers, or hydrolyzed, ring-opened thio-substituted succinimide-including linkers.

In certain embodiments, a linker of the disclosure (L³) comprises a stabilizing linker moiety selected from:

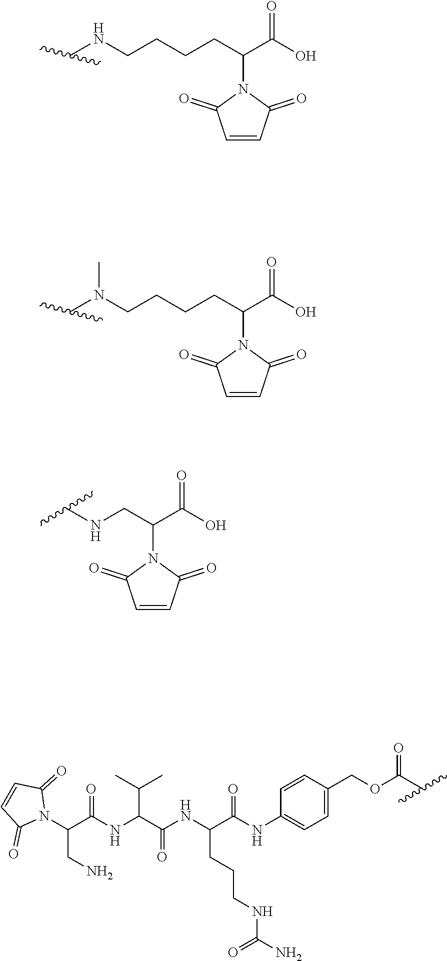 [SMILES: *NCCCCC(C(=O)O)N1C(=O)C=CC1=O ; *N(C)CCCCC(C(=O)O)N1C(=O)C=CC1=O ; *NCC(C(=O)O)N1C(=O)C=CC1=O ; *C(=O)OCc1ccc(NC(=O)C(CCCNC(N)=O)NC(=O)C(NC(=O)C(CN)N2C(=O)C=CC2=O)C(C)C)cc1]

In the scheme provided above, the bottom structure may be referred to as (maleimido)-DPR-Val-Cit-PAB, where DPR refers to diaminopropinoic acid Val refers to valine, Cit refers to citrulline, and PAB refers to para-aminobenzylcarbonyl. [symbol] represents the point of attachment to compound or salt of any one of Formulas (IA), (IB), (IC), (VIIA), (VIIB), or (VIIC).

A method for bridging a pair of sulfhydryl groups derived from reduction of a native hinge disulfide bond has been disclosed and is depicted in the schematic below. An advantage of this methodology is the ability to synthesize homogenous DAR4 conjugates by full reduction of IgGs (to give 4 pairs of sulfhydryls from interchain disulfides) followed by reaction with 4 equivalents of the alkylating agent. Conjugates containing “bridged disulfides” are also claimed to have increased stability.

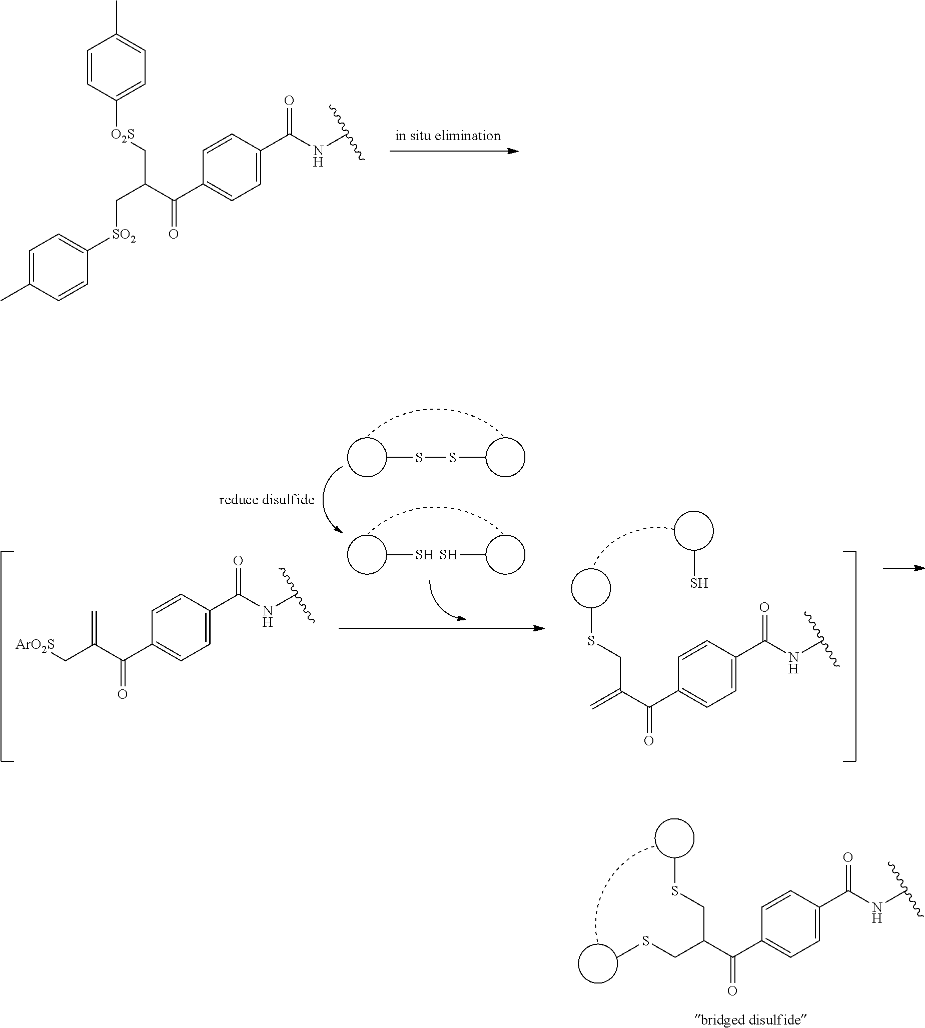

Similarly, as depicted below, a maleimide derivative that is capable of bridging a pair of sulfhydryl groups has been developed.

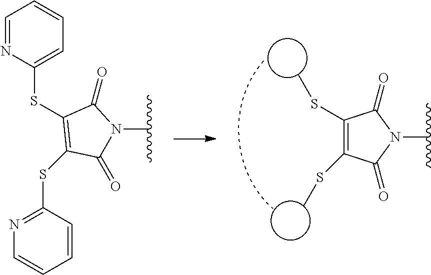 [SMILES: *N1C(=O)C(Sc2ccccn2)=C(Sc2ccccn2)C1=O ; *N1C(=O)C(SC)=C(SC)C1=O]

A linker of the disclosure, L³, can contain the following structural formulas (VIa), (VIb), or (VIc):

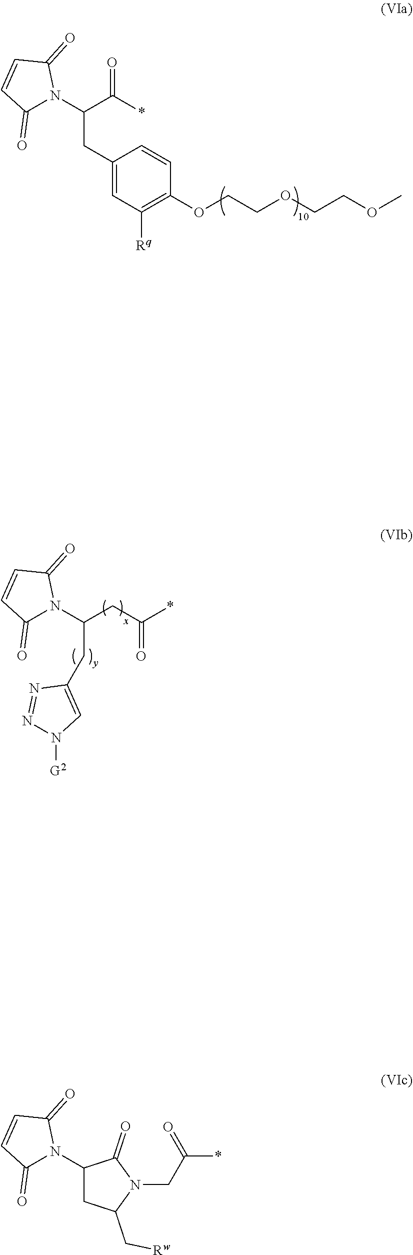

or salts thereof, wherein: R^{q} is H or—O—(CH₂CH₂O)₁₁—CH₃; x is 0 or 1; y is 0 or 1; G² is-CH₂CH₂CH₂SO₃H or—CH₂CH₂O—(CH₂CH₂O)₁₁—CH₃; R^{w} is-O—CH₂CH₂SO₃H or—NH(CO)—CH₂CH₂O—(CH₂CH₂O)₁₂—CH₃; and * represents the point of attachment to the remainder of the linker.

Embodiments of linkers according to structural formula (VIa) and (VIb) that can be included in the conjugates described herein can include the linkers illustrated below (as illustrated, the linkers can include a group suitable for covalently linking the linker to an antibody construct):

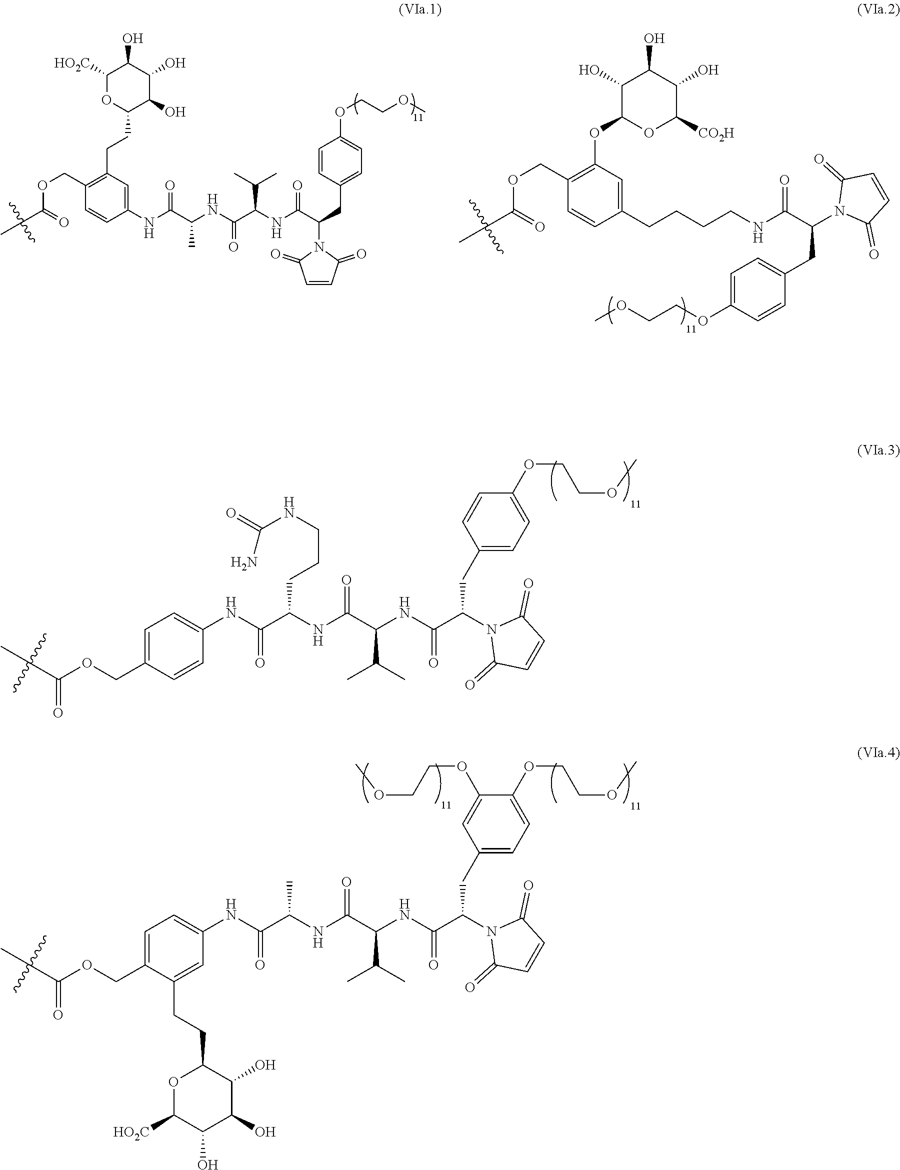

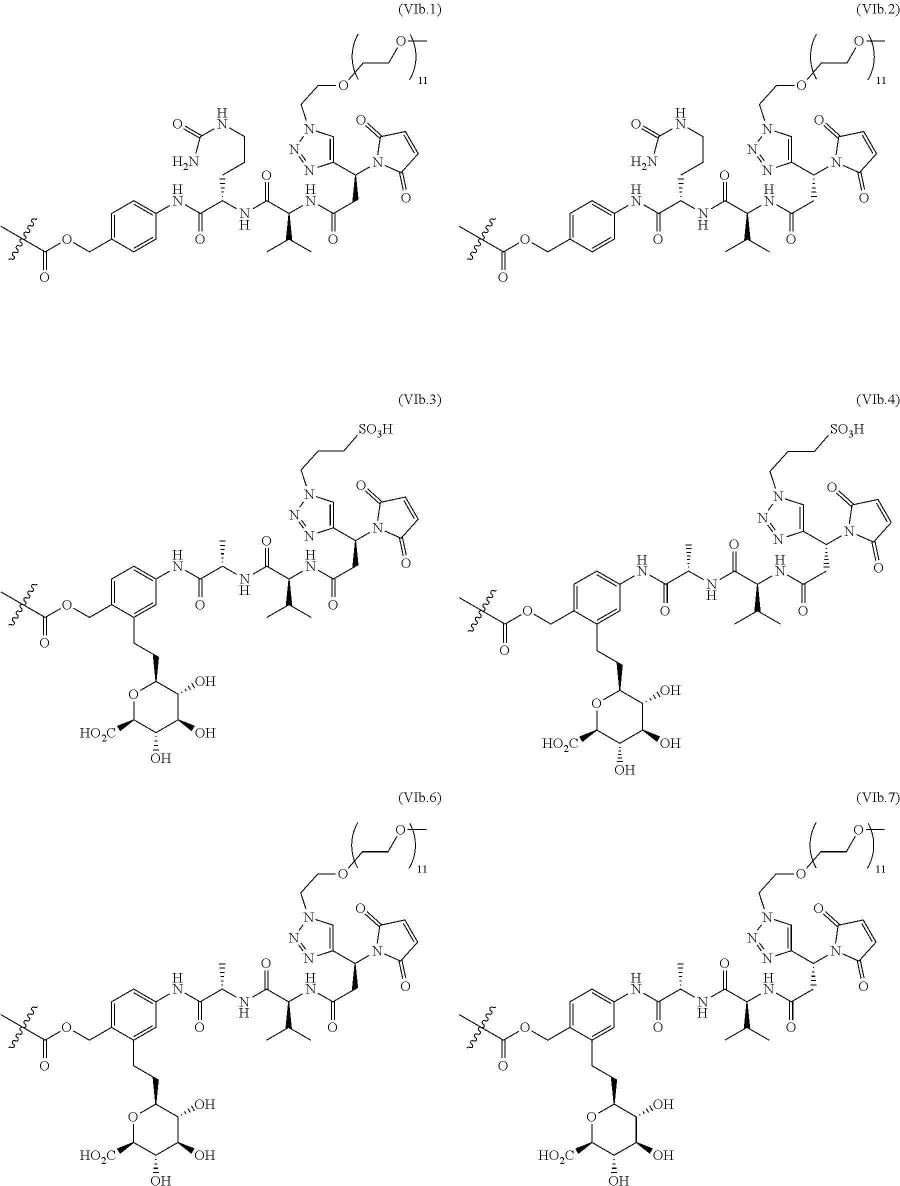

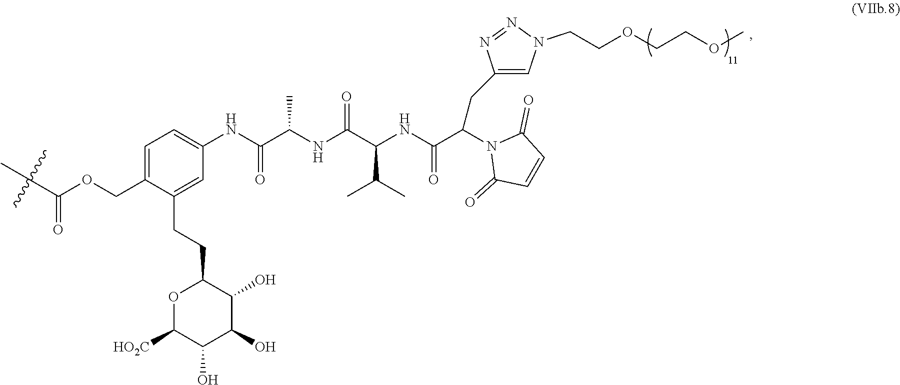

wherein [symbol] represents the point of attachment of the linker (L³) to the compound or salt of any one of Formulas (IA), (IB), (IC), (VIIA), (VIIB), or (VIIC).

Embodiments of linkers according to structural formula (VIc) that can be included in the antibody construct conjugates described herein can include the linkers illustrated below (as illustrated, the linkers can include a group suitable for covalently linking the linker to an antibody construct):

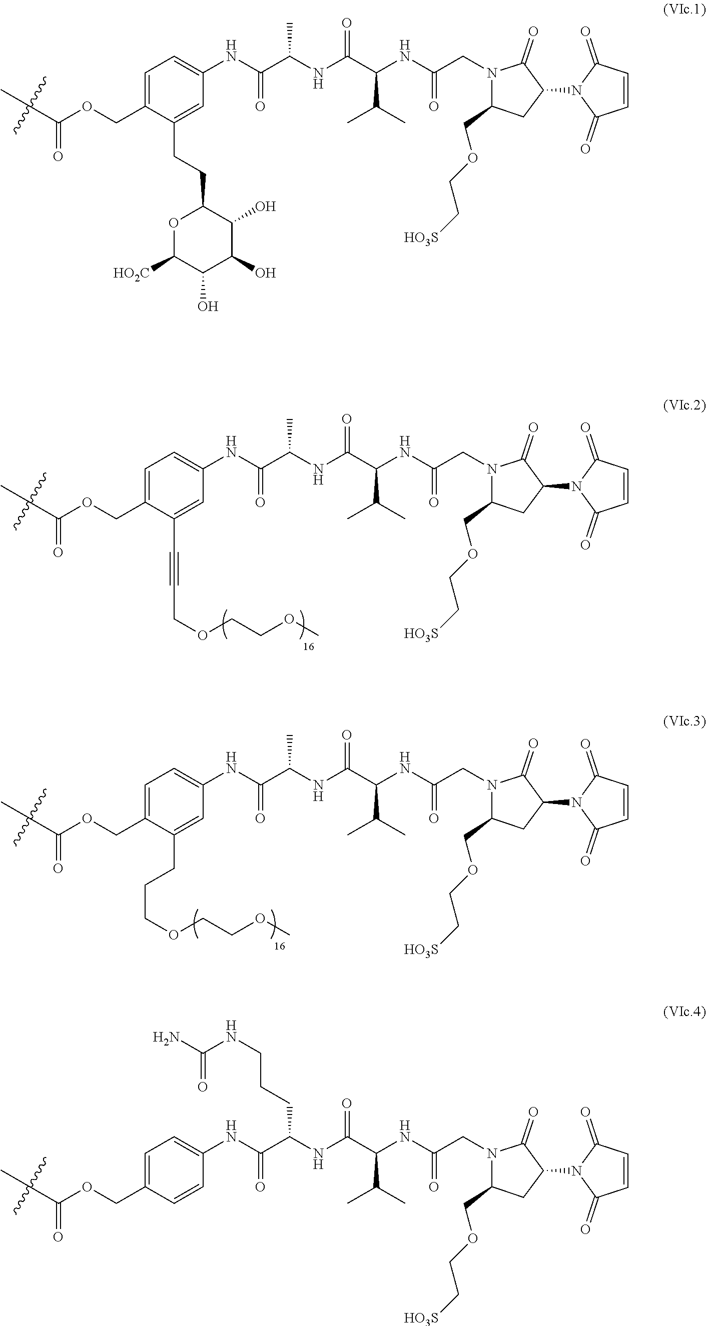

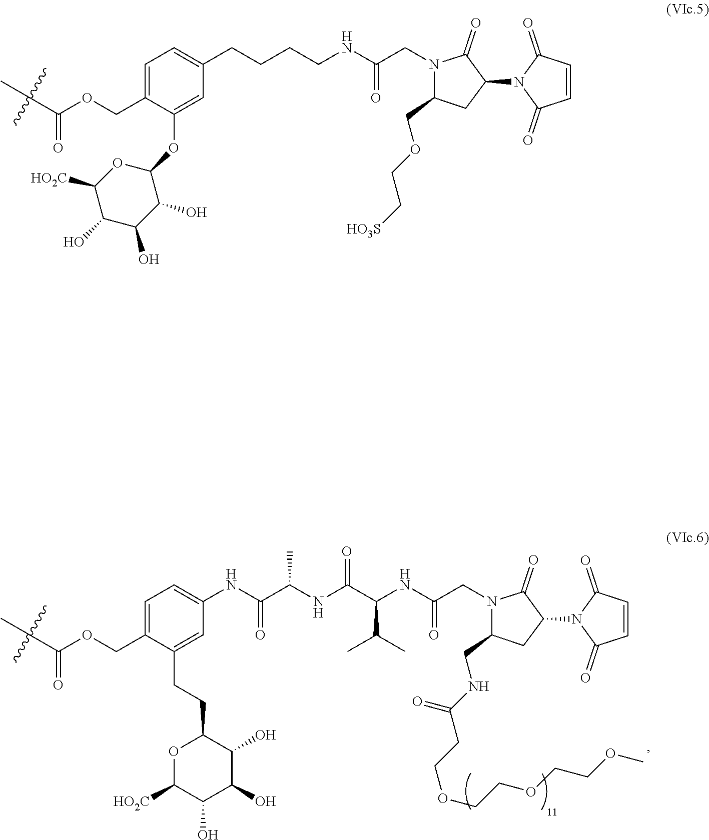

wherein [symbol] represents the point of attachment of the linker (L³) to the compound or salt of any one of Formulas (IA), (IB), (IC), (VIIA), (VIIB), or (VIIC).

The linker selected for a particular conjugate may be influenced by a variety of factors, including but not limited to, the site of attachment to the antibody construct (e.g., lys, cys or other amino acid residues), structural constraints of the drug pharmacophore and the lipophilicity of the drug. The specific linker selected for a conjugate should seek to balance these different factors for the specific antibody construct/drug combination.

For example, conjugates have been observed to effect killing of bystander antigen-negative cells present in the vicinity of the antigen-positive tumor cells. The mechanism of bystander cell killing by conjugates has indicated that metabolic products formed during intracellular processing of the conjugates may play a role. Neutral cytotoxic metabolites generated by metabolism of the conjugates in antigen-positive cells appear to play a role in bystander cell killing while charged metabolites may be prevented from diffusing across the membrane into the medium, or from the medium across the membrane, and therefore cannot affect bystander killing. In certain embodiments, the linker is selected to attenuate the bystander killing effect caused by cellular metabolites of the conjugate. In certain embodiments, the linker is selected to increase the bystander killing effect.

The properties of the linker, or linker-compound, may also impact aggregation of the conjugate under conditions of use and/or storage. Typically, conjugates reported in the literature contain no more than 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than 3-4 could be beneficial as a means of increasing potency. In instances where the imidazoquinoline compound is more hydrophobic in nature, it may be desirable to select linkers that are relatively hydrophilic as a means of reducing conjugate aggregation, especially in instances where DARs greater than 3-4 are desired. Thus, in certain embodiments, the linker incorporates chemical moieties that reduce aggregation of the conjugates during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In particular embodiments, the aggregation of the conjugates during storage or use is less than about 40% as determined by size-exclusion chromatography (SEC). In particular embodiments, the aggregation of the conjugates during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

### Attachment of Linkers to Antibody Construct

The conjugates described herein may comprise a linker, e.g., a cleavable linker such as a peptide linker or a noncleavable linker. Linkers of the conjugates and methods described herein may not affect the binding of active portions of a conjugate (e.g., active portions include antigen binding domains, Fc domains, target binding domains, antibodies, compounds or salts of the disclosed, e.g., Formulas (IA), (IB), (IC), (VIIA), (VIIB), or (VIIC), or linker-compounds of Formulas (IIA), (IIB), (IIC), (VIIIA), (VIIIB), or (VIIIC), to a target, which can be a cognate binding partner such as an antigen. A linker sequence can form a linkage between different parts of a conjugate, e.g., between an antibody construct and a compound or salt of the disclosure. In certain embodiments, a conjugate comprises multiple linkers. In certain embodiments, wherein a conjugate comprises multiple linkers, the linkers may be the same linkers or different linkers.

A linker may be bound to an antibody construct by a bond between the antibody construct and the linker. A linker may be bound to an anti-tumor antigen antibody construct by a bond between the anti-tumor antigen antibody construct and the linker. A linker may be bound to a terminus of an amino acid sequence of an antibody construct, or could be bound to a side chain modification to the antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain or Fc region of an antibody construct, or may be bound to a side chain modification of an Fc domain or Fc region of an antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain of an antibody construct, or may be bound to a side chain modification of an Fc domain of an antibody construct, such as the side chain of a lysine, serine, threonine, cysteine, tyrosine, aspartic acid, glutamine, a non-natural amino acid residue, or glutamic acid residue.

A linker may be bound to an antibody construct at a hinge cysteine. A linker may be bound to an antibody construct at a light chain constant domain lysine. A linker may be bound to an antibody construct at a heavy chain constant domain lysine. A linker may be bound to an antibody construct at an engineered cysteine in the light chain. A linker may be bound to an antibody construct at an Fc region lysine. A linker may be bound to an antibody construct at an Fc domain lysine. A linker may be bound to an antibody construct at an Fc region cysteine. A linker may be bound to an antibody construct at an Fc domain cysteine. A linker may be bound to an antibody construct at a light chain glutamine, such as an engineered glutamine. A linker may be bound to an antibody construct at a heavy chain glutamine, such as an engineered glutamine. A linker may be bound to an antibody construct at an unnatural amino acid engineered into the light chain. A linker may be bound to an antibody construct at an unnatural amino acid engineered into the heavy chain. Amino acids can be engineered into an amino acid sequence of an antibody construct, for example, a linker of a conjugate. Engineered amino acids may be added to a sequence of existing amino acids. Engineered amino acids may be substituted for one or more existing amino acids of a sequence of amino acids.

A linker may be conjugated to an antibody construct via a sulfhydryl group on the antibody construct. A linker may be conjugated to an antibody construct via a primary amine on the antibody construct. A linker may be conjugated to an antibody construct via a residue of an unnatural amino acid on an antibody construct, e.g., a ketone moiety.

When one or more linkers are bound to an antibody construct at the sites described herein, an Fe domain of the antibody construct can bind to Fc receptors. In certain embodiments, an antibody construct bound through a linker or an antibody construct bound to a linker bound to a compound or salt of any one of Formulas (IA), (IB), (IC), (VIIA), (VIIB), or (VIIC), or bound through a compound-linker of Formulas (IIA), (IIB), (IIC), (VIIIA), (VIIIB), or (VIIIC), retains the ability of the Fc domain of the antibody to bind to Fc receptors. In certain embodiments, when a linker is connected to an antibody construct at the sites described herein, the antigen binding domain of an antibody construct bound to a linker or an antibody construct bound through a linker bound to a compound or salt of any one of Formulas (IA), (IB), (IC), (VIIA), (VIIB), or (VIIC), or bound through a compound-linker of Formulas (IIA), (IIB), (IIC), (VIIIA), (VIIIB), or (VIIIC), can bind its antigen. In certain embodiments, when a linker is connected to an antibody construct at the sites described herein, a target binding domain of an antibody construct bound to a linker or an antibody construct bound to a linker bound to a compound or salt of any one of Formulas (IA), (IB), (IC), (VIIA), (VIIB), or (VIIC), can bind its antigen.

In certain embodiments, a compound or a compound-linker of any one of Formulas (IA), (IB), (IC), (VIIA), (VIIB), or (VIIC), disclosed herein is attached to an antibody Fc region or domain at an engineered cysteine residue. In some embodiments, the engineered cysteine residue is at one or more of positions HC S239C, LC V205C, LC A1 14C, HC A140C, LC K149C, LC S168C, LC S153C, LC A127C, HC Ti 16C, and HC S115C, where HC refers to heavy chain, LC refers t light chain and the numbering of amino acid residues in the Fc region is according to the EU index as in Kabat. In certain embodiments, a compound or a compound-linker of any one of Formulas (IA), (IB), (IC), (IIA), (IIB), (IIC), (VIIA), (VIIB), (VIIC), (VIIIA), (VIIIB), or (VIIIC) disclosed herein is attached to an amino acid residue of an IgG Fc domain disclosed herein selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, 428, or any subset thereof wherein numbering of amino acid residues in the Fc domain is according to the EU index as in Kabat.

In certain embodiments, a compound or a compound-linker of any one of Formulas (IA), (IB), (IC), (IIA), (JIB), (IIC), (VIIA), (VIIB), (VIIC), (VIIIA), (VIIIB), or (VIIIC) disclosed herein may not be attached to an amino acid residue of an IgG Fc domain disclosed herein selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, 428, or any subset thereof wherein numbering of amino acid residues in the Fe domain is according to the EU index as in Kabat.

### Lysine-Based Bioconjugation

An antibody construct can be conjugated to a linker via lysine-based bioconjugation. An antibody construct can be exchanged into an appropriate buffer, for example, phosphate, borate, PBS, Tris-Acetate, Tris-Glycine, HEPES, MOPS, MES, EPS, HEPPS, Histidine, or HEPBS at a concentration of about 2 mg/mL to about 10 mg/mL. An appropriate number of equivalents of a compound-linker, e.g., a compound or salt of Formula (IIA), (IIB), (IIC), (VIIIA), (VIIIB), or (VIIIC), described herein can be added as a solution with stirring. Dependent on the physical properties of the compound-linker construct, a co-solvent can be introduced prior to the addition of the compound-linker construct to facilitate solubility. The reaction can be stirred at room temperature for about 2 hours to about 12 hours depending on the observed reactivity. The progression of the reaction can be monitored by LC-MS. Once the reaction is deemed complete, the remaining compound-linker constructs can be removed by applicable methods and the antibody construct-conjugate can be exchanged into the desired formulation buffer. Lysine-linked conjugates can be synthesized starting with antibody (mAb) or bispecific antibody (bsAb) and compound-linker construct, e.g., 10 equivalents, following Scheme A below (Conjugate=antibody construct-compound conjugate). Monomer content and compound-antibody construct ratios (molar ratios) can be determined by methods described herein.

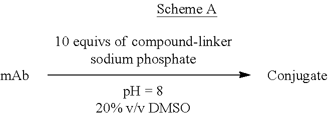

### Cysteine-Based Bioconjugation

An antibody construct can be conjugated to a linker via cysteine-based bioconjugation. An antibody construct can be exchanged into an appropriate buffer, for example, phosphate, borate, PBS, Tris-Acetate, Tris-Glycine, HEPES, MOPS, MES, EPS, HEPPS, Histidine, or HEPBS at a concentration of about 2 mg/mL to about 10 mg/mL with an appropriate number of equivalents of a reducing agent, for example, dithiothreitol or tris(2-carboxyethyl)phosphine. The resultant solution can be stirred for an appropriate amount of time and temperature to effect the desired reduction. A compound-linker, e.g., a compound or salt of Formula (IIA), (IIB), (IIC), (VIIIA), (VIIIB), or (VIIIC), described herein can be added as a solution with stirring. Dependent on the physical properties of the compound

-linker construct, a co-solvent can be introduced prior to the addition of the compound-linker construct to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free compound-linker construct can be removed by applicable methods and the antibody construct-conjugate can be exchanged into the desired formulation buffer. Such cysteine-based conjugates can be synthesized starting with antibody (mAb) and compound-linker construct, e.g., 7 equivalents, using the conditions described in Scheme B below (Conjugate=antibody construct-conjugate). Monomer content and drug-antibody ratios can be determined by methods described herein.

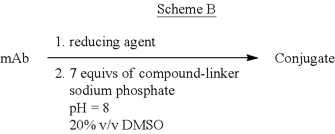

### Pharmaceutical Formulations

The compositions, conjugates and methods described herein can be considered useful as pharmaceutical compositions for administration to a subject in need thereof. Pharmaceutical compositions can comprise at least the compounds, salts or conjugates described herein and one or more pharmaceutically acceptable carriers, diluents, excipients, stabilizers, dispersing agents, suspending agents, and/or thickening agents. The composition can comprise the conjugate having an antibody construct and a compound or salt of any one of Formulas (IA), (IB), (IC), (VIIA), (VIIB), or (VIIC) connected via a linker, as described herein. The composition can comprise the conjugate having an antibody construct, a target binding domain, and a compound or salt of any one of Formulas (IA), (IB), (IC), (VIIA), (VIIB), or (VIIC) connected via a linker. The composition can comprise any conjugate described herein. The antibody construct can be an anti-CD40 antibody. A conjugate can comprise an anti-CD40 antibody and a compound of Formulas (IA), (IB), (IC), (VIIA), (VIIB), or (VIIC). A conjugate can comprise an anti-HER2 antibody and a imidazoquinoline. A conjugate can comprise an anti-TROP2 antibody and a compound of Formulas (IA), (IB), (IC), (VIIA), (VIIB), or (VIIC). A pharmaceutical composition can comprise at least the compounds, salts or conjugates described herein and one or more of buffers, antibiotics, steroids, carbohydrates, drugs (e.g., chemotherapy drugs), radiation, polypeptides, chelators, adjuvants and/or preservatives.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate as described herein can be manufactured, for example, by lyophilizing the compound, salt or conjugate, mixing, dissolving, emulsifying, encapsulating or entrapping the conjugate. The pharmaceutical compositions can also include the compounds, salts or conjugates described herein in a free-base form or pharmaceutically-acceptable salt form.

Methods for formulation of the conjugates described herein can include formulating any of the compounds, salts or conjugates described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates described herein can be lyophilized or in powder form for re-constitution with a suitable vehicle, e.g., sterile pyrogen-free water, before use.

Pharmaceutical compositions described herein can comprise at least one active ingredient (e.g., a compound, salt or conjugate). The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein often further can comprise more than one active compound (e.g., a compound, salt or conjugate and other agents) as necessary for the particular indication being treated. The active compounds can have complementary activities that do not adversely affect each other. For example, the composition can also comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The compositions and formulations can be sterilized. Sterilization can be accomplished by filtration through sterile filtration.

The compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions described herein can be lyophilized or in powder form for reconstitution with a suitable vehicle, e.g., sterile pyrogen-free water, before use.

For parenteral administration, the compounds, salts or conjugates can be formulated in a unit dosage injectable form (e.g., use letter solution, suspension, emulsion) in association with a pharmaceutically acceptable parenteral vehicle. Such vehicles can be inherently non-toxic, and non-therapeutic. Vehicles can be water, saline, Ringer's solution, dextrose solution, and 5% human serum albumin. Non-aqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the compound, salt or conjugate, and these matrices can be in the form of shaped articles (e.g., films or microcapsules). Examples of sustained-release matrices can include polyesters, hydrogels (e.g., poly(2-hydroxyethyl-methacrylate), or poly (vinyl alcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations described herein can be prepared for storage by mixing a compound, salt or conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Pharmaceutical formulations of the conjugates described herein may have an average drug-antibody construct ratio (“DAR”) selected from about 1 to about 10, wherein the drug is a compound or salt of any one of Formulas (IA), (IB), (IC), (VIIA), (VIIB), or (VIIC). In certain embodiments, the average DAR of the formulation is from about 2 to about 8, such as from about 3 to about 8, such as from about 3 to about 7. In certain embodiments, a pharmaceutical formulation has an average DAR of about 2, about 3, about 3.5, about 4, about 4.5, about 5, about 5.5, about 6, or about 6.6.

### Therapeutic Applications

The compositions, conjugates and methods of the present disclosure can be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The compositions, conjugates and methods described herein can be useful as a therapeutic, for example, a treatment that can be administered to a subject in need thereof. A therapeutic effect of the present disclosure can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the compositions, and conjugates described herein can be administered to a subject in need thereof, often for treating and/or preventing a condition or progression thereof. A pharmaceutical composition can affect the physiology of the subject, such as the immune system, an inflammatory response, or other physiologic affect. A therapeutically-effective amount can vary depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like can refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual. Preventing can also refer to preventing re-occurrence of a disease or condition in a patient that has previously been treated for the disease or condition, e.g., by preventing relapse.

A therapeutically effective amount can be the amount of a composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The conjugates described herein that can be used in therapy can be formulated and dosages established in a fashion consistent with good medical practice taking into account the disease or condition to be treated, the condition of the individual patient, the site of delivery of the composition, the method of administration and other factors known to practitioners. The compositions described herein can be prepared according to the description of preparation described herein.

Pharmaceutical compositions can be used in the methods described herein and can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods and compositions described herein can be for administration to a subject in need thereof. Often, administration of the compositions described herein can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition can be administered to a subject by additional routes of administration, for example, by inhalation, oral, dermal, intranasal, or intrathecal administration.

Compositions and conjugates of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month. The administrations can be weekly, biweekly (every two weeks), every three weeks, monthly or bimonthly.

The compositions, conjugates and methods provided herein can be useful for the treatment of a plurality of diseases, conditions, preventing a disease or a condition in a subject or other therapeutic applications for subjects in need thereof. Often the compositions, conjugates and methods provided herein can be useful for treatment of hyperplastic conditions, including but not limited to, neoplasms, cancers, tumors and the like. The compositions, conjugates and methods provided herein can be useful for specifically targeting TLR7. In one embodiment, the compounds of the present disclosure serve as TLR7 agonists and activate an immune response. In another embodiment, the conjugates of the present disclosure serve as TLR7 agonists and activate an immune response. A condition, such as a cancer, can be associated with expression of a molecule on the cancer cells. Often, the molecule expressed by the cancer cells can comprise an extracellular portion capable of recognition by the antibody construct of the conjugate. A molecule expressed by the cancer cells can be a tumor antigen. An antibody construct portion of the conjugate can recognize a tumor antigen. A tumor antigen can include CD5, CD19, CD20, CD25, CD37, CD30, CD33, CD40, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PMSA), ferritin, GD2, GD3, GM2, Le^{y}, CA-125, CA19-9, epidermal growth factor, p185HER2, IL-2 receptor, EGFRvIII (de2-7 EGFR), fibroblast activation protein, tenascin, a metalloproteinase, endosialin, vascular endothelial growth factor, avB3, WT1, LMP2, HPV E6, HPV E7, Her-2/neu, MAGE A3, p53 nonmutant, NY-ESO-1, MelanA/MART1, Ras mutant, gp100, p53 mutant, PR1, ber-abl, tyronsinase, survivin, PSA, hTERT, a Sarcoma translocation breakpoint protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, Legumain, Tie 3, VEGFR2, MAD-CT-1, PDGFR-B, MAD-CT-2, ROR2, TRAIL1, MUC16, MAGE A4, MAGE C2, GAGE, EGFR, CMET, HER3, MUC15, CA6, NAPI2B, TROP2, CLDN6, CLDN16, CLDN18.2, CLorfl86, RON, LY6E, FRA, DLL3, PTK7, STRA6, TMPRSS3, TMPRSS4, TMEM238, UPK1B, VTCN1, LIV1, ROR1, or Fos-related antigen 1.

In certain embodiments, a tumor antigen is selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

As described herein, an antigen binding domain portion of the conjugate may be configured to recognize a molecule expressed by a cancer cell, such as for example, a disease antigen, tumor antigen or a cancer antigen. Often such antigens are known to those of ordinary skill in the art, or newly found to be associated with such a condition, to be commonly associated with, and/or, specific to, such conditions. For example, a disease antigen, tumor antigen or a cancer antigen is, but is not limited to, CD5, CD19, CD20, CD25, CD37, CD30, CD33, CD40, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, EGFRVIII (de2-7 EGFR), fibroblast activation protein, tenascin, a metalloproteinase, endosialin, vascular endothelial growth factor, avB3, WT1, LMP2, HPV E6, HPV E7, Her-2/neu, MAGE A3, p53 nonmutant, NY-ESO-1, MelanA/MART1, Ras mutant, gp100, p53 mutant, PR1, bcr-abl, tyronsinase, survivin, PSA, hTERT, a Sarcoma translocation breakpoint protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, Legumain, Tie 3, VEGFR2, MAD-CT-1, PDGFR-B, MAD-CT-2, ROR2, TRAIL1, MUC16, MAGE A4, MAGE C2, GAGE, EGFR, CMET, HER3, MUC15, CA6, NAPI2B, TROP2, CLDN6, CLDN16, CLDN18.2, CLorfl86, RON, LY6E, FRA, DLL3, PTK7, STRA6, TMPRSS3, TMPRSS4, TMEM238, UPK1B, VTCN1, LIV1, ROR1, or Fos-related antigen 1. In another example, a disease antigen, tumor antigen or a cancer antigen is selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1. Additionally, such tumor antigens can be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

Non-limiting examples of cancers can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sezary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenström macroglobulinemia; Wilms tumor and any combination thereof.

The disclosure provides any therapeutic compound or conjugate disclosed herein for use in a method of treatment of the human or animal body by therapy. Therapy may be by any mechanism disclosed herein, such as by stimulation of the immune system. The disclosure provides any therapeutic compound or conjugate disclosed herein for use in stimulation of the immune system, vaccination or immunotherapy, including for example enhancing an immune response. The disclosure further provides any therapeutic compound or conjugate disclosed herein for prevention or treatment of any condition disclosed herein, for example cancer, autoimmune disease, inflammation, sepsis, allergy, asthma, graft rejection, graft-versus-host disease, immunodeficiency or infectious disease (typically caused by an infectious pathogen). The disclosure also provides any therapeutic compound or conjugate disclosed herein for obtaining any clinical outcome disclosed herein for any condition disclosed herein, such as reducing tumour cells in vivo. The disclosure also provides use of any therapeutic compound or conjugate disclosed herein in the manufacture of a medicament for preventing or treating any condition disclosed herein.

### General Synthetic Schemes and Examples

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

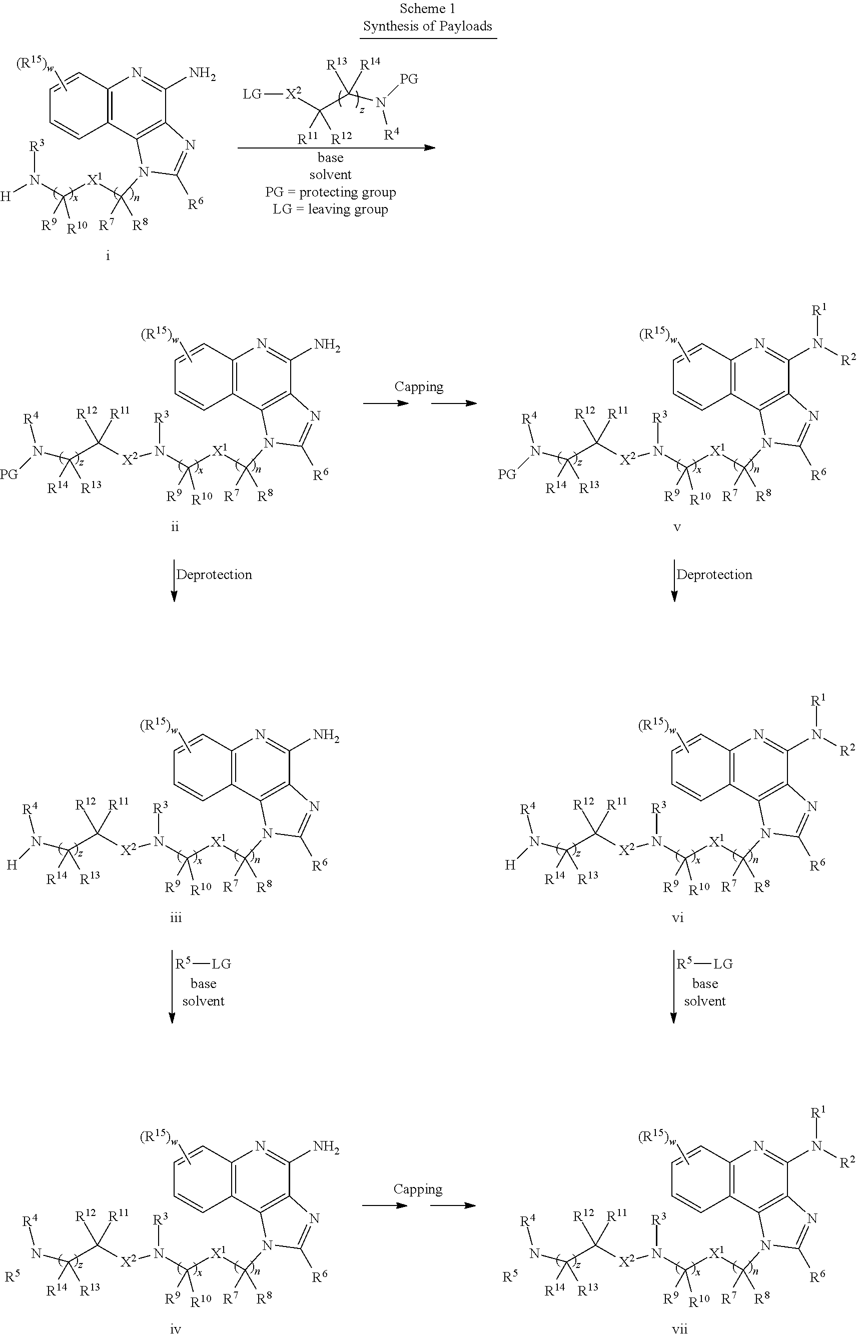

A 4-amino imidazoquinoline (i) with a pendent amino-functionality may be acylated, or alkylated, when treated with an appropriate electrophile in the presence of an appropriate base in an appropriate solvent, to give compounds of type (ii). Subsequent deprotection of a protecting group (PG), if applicable, results in the generation of compound (iii), containing a free amine which may be functionalized in an analogous fashion to the first step of this sequence (i→ii). Alternatively, the 4-amino compound (ii) may be capped via treatment with an appropriate electrophile to provide access to compounds of type (v). Compounds of type (v) can be converted to compounds of type (vii) just as compounds of type (ii) are converted to (iv). In some instances, compounds of type (iv) may be modified directly to access compounds of type (vii), via treatment with an appropriate electrophile in the presence of an appropriate base in an appropriate solvent.

### Example 1

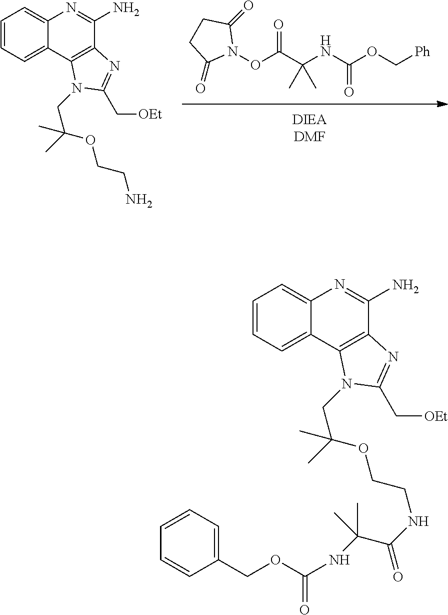 [SMILES: CCOCc1nc2c(N)nc3ccccc3c2n1CC(C)(C)OCCN ; CC(C)(NC(=O)OCc1ccccc1)C(=O)ON1C(=O)CCC1=O ; CCOCc1nc2c(N)nc3ccccc3c2n1CC(C)(C)OCCNC(=O)C(C)(C)NC(=O)OCc1ccccc1]

### Compound 1.1: Benzyl (1-((2-((1-(4-amino-2-(ethoxymethyl)-1H-imidazo[4,5-c]quinolin-1-yl)-2-methylpropan-2-yl)oxy)ethyl)amino)-2-methyl-1-oxopropan-2-yl)carbamate

To a mixture of 1-(2-(2-aminoethoxy)-2-methylpropyl)-2-(ethoxymethyl)-1H-imidazo[4,5-c]quinolin-4-amined (100 mg, 0.28 mmol) and diisopropylethylamine (3.0 equiv., 146 μL, 0.84 mmol) in DMF (5 mL) was added 2,5-dioxopyrrolidin-1-yl 2-(((benzyloxy)carbonyl)amino)-2-methylpropanoate (1.2 equiv., 112 mg, 0.33 mmol). The mixture was stirred at RT for 1h. The crude reaction was purified via preparative RP-HPLC (0→100% AcN in H₂O, 0.1% TFA). Pure fractions were pooled, frozen and dried via lyophilization to give 141 mg (91% yield) of the desired product as an off-white solid. LCMS (M+H)=577.7. ¹H NMR (DMSO, 400 MHz) δ 13.71 (s, 1H), 8.53 (d, 1H, J=8.4 Hz), 7.78 (dd, 1H, J=8.0, 0.8 Hz), 7.68 (dd, 1H, J=7.6, 0.8), 7.53 (td, 1H, J=8.4, 1.2 Hz), 7.38-7.27 (m, 7H), 7.28-7.17 (m, 1H), 4.95 (s, 3H), 3.55 (q, 2H, 6.8 Hz), 3.18-3.08 (m, 2H), 2.95-2.90 (m, 1H), 1.23 (s, 3H), 1.62 (bs, 6H), 1.14 (t, 3H, J=6.8 Hz).

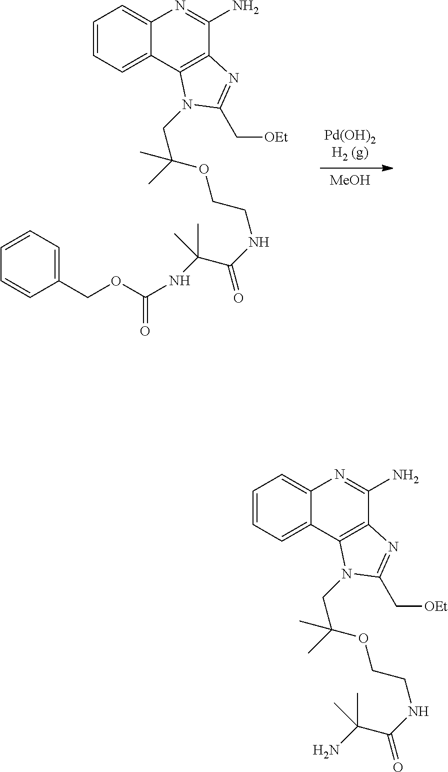 [SMILES: "MeOH" ; CCOCc1nc2c(N)nc3ccccc3c2n1CC(C)(C)OCCNC(=O)C(C)(C)NC(=O)OCc1ccccc1 ; CCOCc1nc2c(N)nc3ccccc3c2n1CC(C)(C)OCCNC(=O)C(C)(C)N]

### Compound 1.2: 2-amino-N-(2-((1-(4-amino-2-(ethoxymethyl)-1H-imidazo[4,5-c]quinolin-1-yl)-2-methylpropan-2-yl)oxy)ethyl)-2-methylpropanamide

To a solution of benzyl (1-((2-((1-(4-amino-2-(ethoxymethyl)-1H-imidazo[4,5-c]quinolin-1-yl)-2-methylpropan-2-yl)oxy)ethyl)amino)-2-methyl-1-oxopropan-2-yl)carbamate 1.1 (130 mg, 0.23 mmol) in MeOH (20 mL) was added Pd(OH)₂ (30 mg). The reaction was then placed under an atmosphere of H₂ (g) and stirred vigorously, at ambient temperature, for 3h. The reaction mixture was then filtered through a plug of celite, rinsed with MeOH, and concentrated. The resulting residue was purified via preparative RP-HPLC (0→100% AcN in H₂O, 0.1% TFA). Pure fractions were pooled, frozen and dried via lyophilization to give 123.5 mg (94% yield) of the desired product (TFA salt) as an off-white solid. C₂₃H₃₄N₆O₃ LCMS (M+H)=443.6. ¹H NMR (DMSO, 400 MHz) δ 14.08 (bs, 1H), 9.14 (bs, 2H), 8.53 (d, 1H, J=8.0 Hz), 8.08 (bs, 3H), 8.03 (t, 1H, J=5.6 Hz), 7.78 (dd, 1H, J=8.4, 1.2 Hz), 7.70 (td, 1H, J=7.2, 1.2 Hz), 7.58 (td, 1H, J=7.2, 1.2 Hz), 4.84 (bs, 4H), 3.54 (q, 3H, J=6.8 Hz), 3.23 (t, 2H, J=6.4 Hz), 2.96 (m, 2H), 1.35 (s, 3H), 1.19 (bs, 3H), 1.13 (t, 3H, J=6.8 Hz).

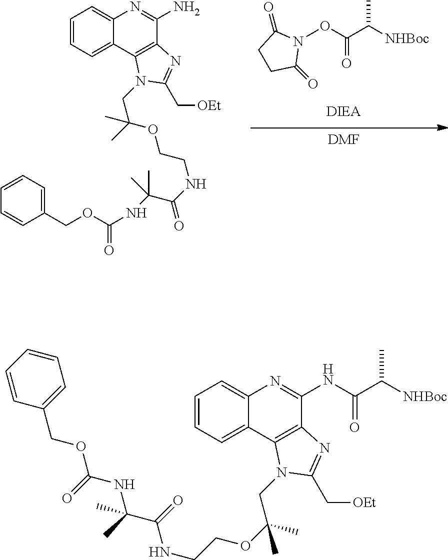

### Compound 1.3: Benzyl (S)-(1-((2-((1-(4-(2-((tert-butoxycarbonyl)amino)propanamido)-2-(ethoxymethyl)-1H-imidazo[4,5-c]quinolin-1-yl)-2-methylpropan-2-yl)oxy)ethyl)amino)-2-methyl-1-oxopropan-2-yl)carbamate

To a mixture of benzyl (1-((2-((1-(4-amino-2-(ethoxymethyl)-1H-imidazo[4,5-c]quinolin-1-yl)-2-methylpropan-2-yl)oxy)ethyl)amino)-2-methyl-1-oxopropan-2-yl)carbamate 1.1 (130 mg, 0.22 mmol) and diisopropylethylamine (4.0 equiv., 166 μL, 0.90 mmol) in DMF (7.5 mL) was added 2,5-dioxopyrrolidin-1-yl (tert-butoxycarbonyl)-L-alaninate (2.0 equiv., 135 mg, 0.45 mmol). The mixture was stirred at 40° C. for 15h. The crude reaction was purified via preparative RP-HPLC (0→100% AcN in H₂O, 0.1% TFA). Pure fractions were pooled, frozen and dried via lyophilization to give 112 mg (65% yield) of the desired product as an off-white solid. LCMS (M+Na)=769.9, (M+H−Boc)=647.9

Other compounds of the same class described above may be prepared in a manner similar to that described in Example 1 above using the appropriate reagents.

TABLE 1
Compounds 1.1-1.9

| Compound | Structure | Spectroscopic Data |
|---|---|---|
| 1.1 | 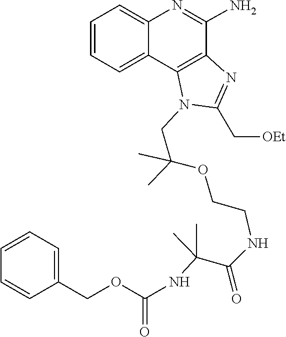 [SMILES: CCOCc1nc2c(N)nc3ccccc3c2n1CC(C)(C)OCCNC(=O)C(C)(C)NC(=O)OCc1ccccc1] | ¹H NMR (DMSO, 400 MHz) δ 13.71 (s, 1H), 8.53 (d, 1H, J = 8.4 Hz), 7.78 (dd, 1H, J = 8.0, 0.8 Hz), 7.68 (dd, 1H, J = 7.6, 0.8), 7.53 (td, 1H, J = 8.4, 1.2 Hz), 7.38-7.27 (m, 7H), 7.28-7.17 (m, 1H), 4.95 (s, 3H), 3.55 (q, 2H, 6.8 Hz), 3.18-3.08 (m, 2H), 2.95- 2.90 (m, 1H), 1.23 (s, 3H), 1.62 (bs, 6H), 1.14 (t, 3H, J = 6.8 Hz). LCMS (M + H) = 577.7. |
| | benzyl (1-((2-((1-(4-amino-2-(ethoxymethyl)-1H-imidazo[4,5-c]quinolin-1-yl)-2- | |
| | methylpropan-2-yl)oxy)ethyl)amino)-2-methyl-1-oxopropan-2-yl)carbamate | |
| 1.2 | 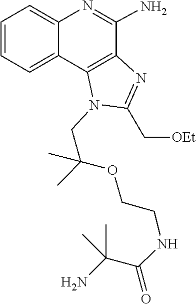 [SMILES: CCOCc1nc2c(N)nc3ccccc3c2n1CC(C)(C)OCCNC(=O)C(C)(C)N] | ¹H NMR (DMSO, 400 MHz) δ 14.08 (bs, 1H), 9.14 (bs, 2H), 8.53 (d, 1H, J = 8.0 Hz), 8.08 (bs, 3H), 8.03 (t, 1H, J = 5.6 Hz), 7.78 (dd, 1H, J = 8.4, 1.2 Hz), 7.70 (td, 1H, J = 7.2, 1.2 Hz), 7.58 (td, 1H, J = 7.2, 1.2 Hz), 4.84 (bs, 4H), 3.54 (q, 3H, J = 6.8 Hz), 3.23 (t, 2H, J = 6.4 Hz), 2.96 (m, 2H), 1.35 (s, 3H), 1.19 (bs, 3H), 1.13 (t, 3H, J = 6.8 Hz). LCMS (M + H) = 443.6. |
| | 2-amino-N-(2-((1-(4-amino-2-(ethoxymethyl)-1H-imidazo[4,5-c]quinolin-1-yl)-2- | |
| | methylpropan-2-yl)oxy)ethyl)-2-methylpropanamide | |
| 1.3 | 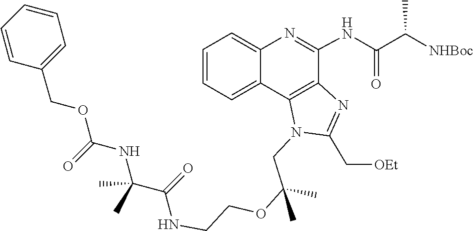 | LCMS (M + Na) = 769.9, (M + H − Boc) = 647.9 |
| | benzyl (S)-(1-((2-((1-(4-(2-((tert-butoxycarbonyl)amino)propanamido)-2- | |
| | (ethoxymethyl)-1H-imidazo[4,5-c]quinolin-1-yl)-2-methylpropan-2- | |
| | yl)oxy)ethyl)amino)-2-methyl-1-oxopropan-2-yl)carbamate | |
| 1.4 | 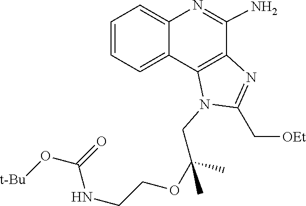 | LCMS (M + Na) = 478.6, (M + H − Boc) = 356.6 |
| | tert-butyl (2-((1-(4-amino-2-(ethoxymethyl)-1H-imidazo[4,5- | |
| | c]quinolin-1-yl)-2-methylpropan-2-yl)oxy)ethyl)carbamate | |
| 1.5 | 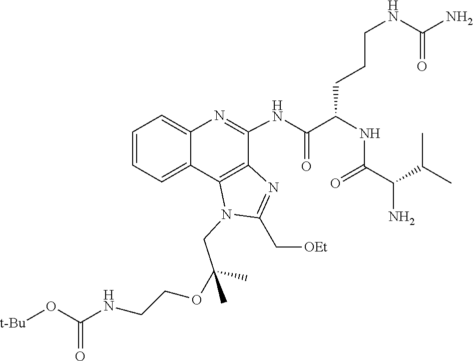 | LCMS (M + Na) = 736.9, (M + H − Boc) = 614.9 |
| | tert-butyl (2-((1-(4-((S)-2-((S)-2-amino-3-methylbutanamido)-5-ureidopentanamido)-2- | |
| | (ethoxymethyl)-1H-imidazo[4,5-c]quinolin-1-yl)-2-methylpropan-2-yl)oxy)ethyl)carbamate | |
| 1.6 | 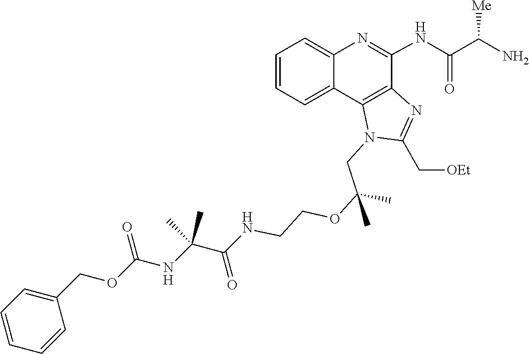 | LCMS (M + H) = 648.8 |
| | benzyl (S)-(1-((2-((1-(4-(2-aminopropanamido)-2-(ethoxymethyl)-1H-imidazo[4,5- | |
| | c]quinolin-1-yl)-2-methylpropan-2-yl)oxy)ethyl)amino)-2-methyl-1- | |
| | oxopropan-2-yl)carbamate | |
| 1.7 | 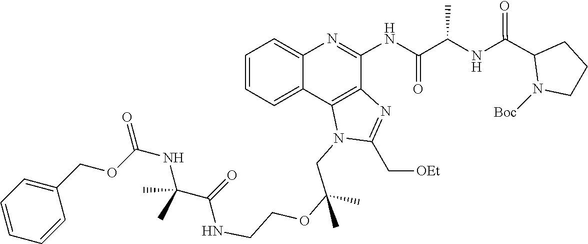 | LCMS (M + H) = 686.0 |
| | tert-butyl 2-(((S)-1-((2-(ethoxymethyl)-1-(5,5,11,11-tetramethyl-3,6-dioxo-1-phenyl-2,10- | |
| | dioxa-4,7-diazadodecan-12-yl)-1H-imidazo[4,5-c]quinolin-4-yl)amino)-1-oxopropan-2- | |
| | yl)carbamoyl)pyrrolidine-1-carboxylate | |
| 1.8 | 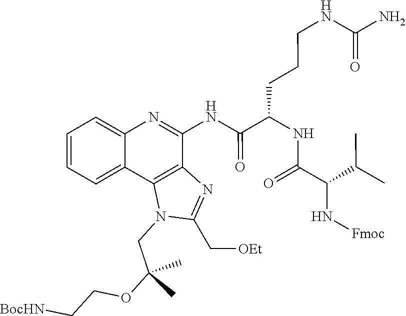 | LCMS (M + Na) = 958.1, (M + H − Boc) = 836.0 |
| | (9H-fluoren-9-yl)methyl ((S)-1-(((S)-1-((1-(2-(2-((tert-butoxycarbonyl)amino)ethoxy)-2- | |
| | methylpropyl)-2-(ethoxymethyl)-1H-imidazo[4,5-c]quinolin-4-yl)amino)-1-oxo- | |
| | 5-ureidopentan-2-yl)amino)-3-methyl-1-oxobutan-2-yl)carbamate | |
| 1.9 | 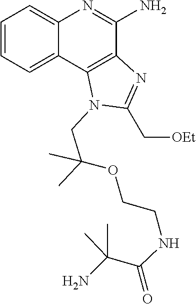 [SMILES: CCOCc1nc2c(N)nc3ccccc3c2n1CC(C)(C)OCCNC(=O)C(C)(C)N] | LCMS (M + H) = 443.6 |
| | 2-amino-N-(2-((1-(4-amino-2-(ethoxymethyl)-1H-imidazo[4,5-c]quinolin- | |
| | 1-yl)-2-methylpropan-2-yl)oxy)ethyl)-2-methylpropanamide | |

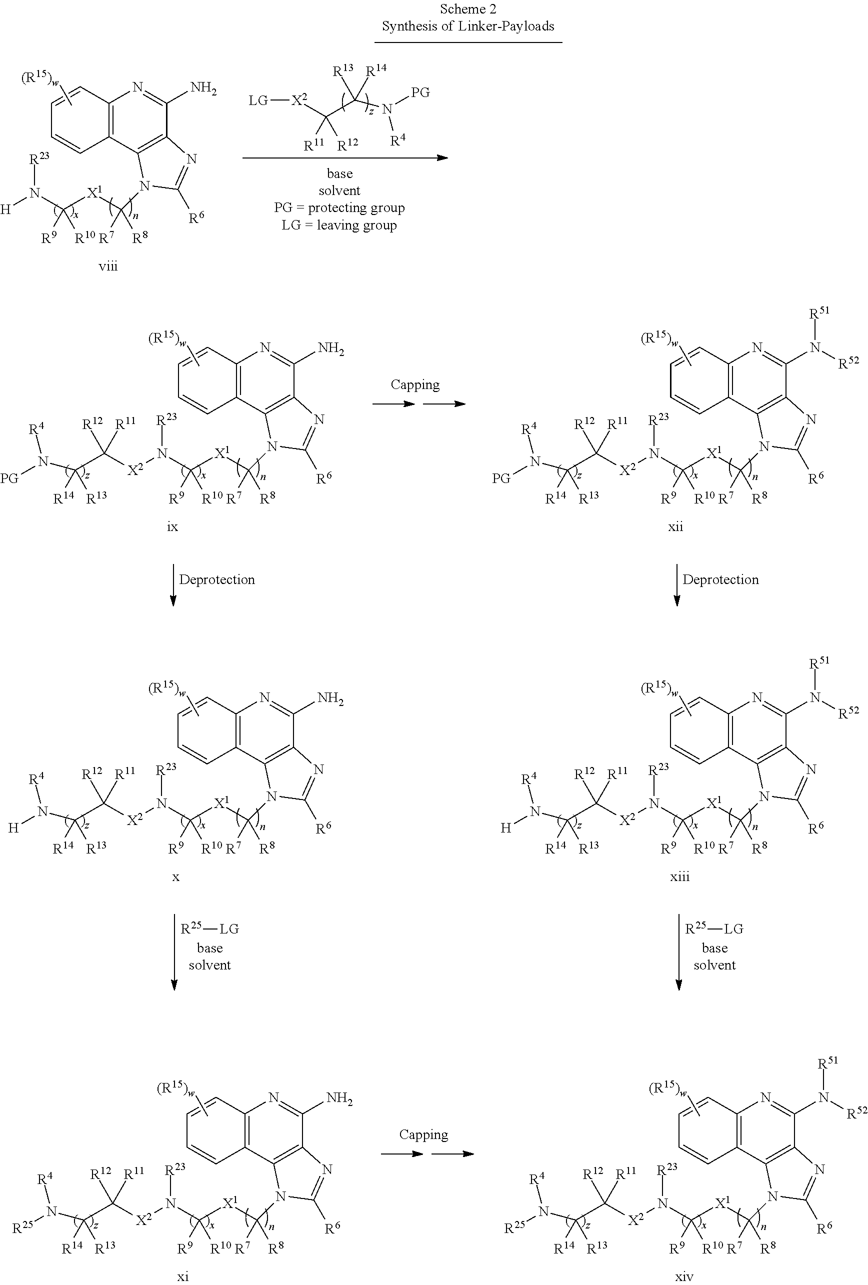

A linker-payload (LP) can be synthesized by various methods. For example, LP compounds can be synthesized as shown in Scheme 2.

A 4-amino imidazoquinoline (viii) with a pendent amino-functionality may be acylated, or alkylated, when treated with an appropriate electrophile in the presence of an appropriate base in an appropriate solvent, to give compounds of type (ix). Subsequent deprotection of a protecting group (PG), if applicable, results in the generation of compound (x), containing a free amine to which may be functionalized in an analogous fashion to the first step of this sequence (i→ii), R²⁵ can be defined as a linker: L³. In some instances, compounds of type (xi) may be modified directly to access compounds of type (xiv), via treatment with an appropriate electrophile in the presence of an appropriate base in an appropriate solvent.

A linker-payload (LP), additionally, can be synthesized by various methods. For example, LP compounds can be synthesized as shown in Scheme 3-1.

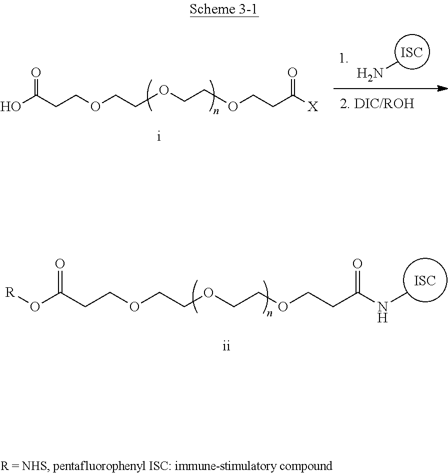

A PEGylated carboxylic acid (i) that has been activated for amide bond formation can be reacted with an appropriately substituted amine containing immune-stimulatory compound to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

An LP can be synthesized as shown in Scheme 3-2.

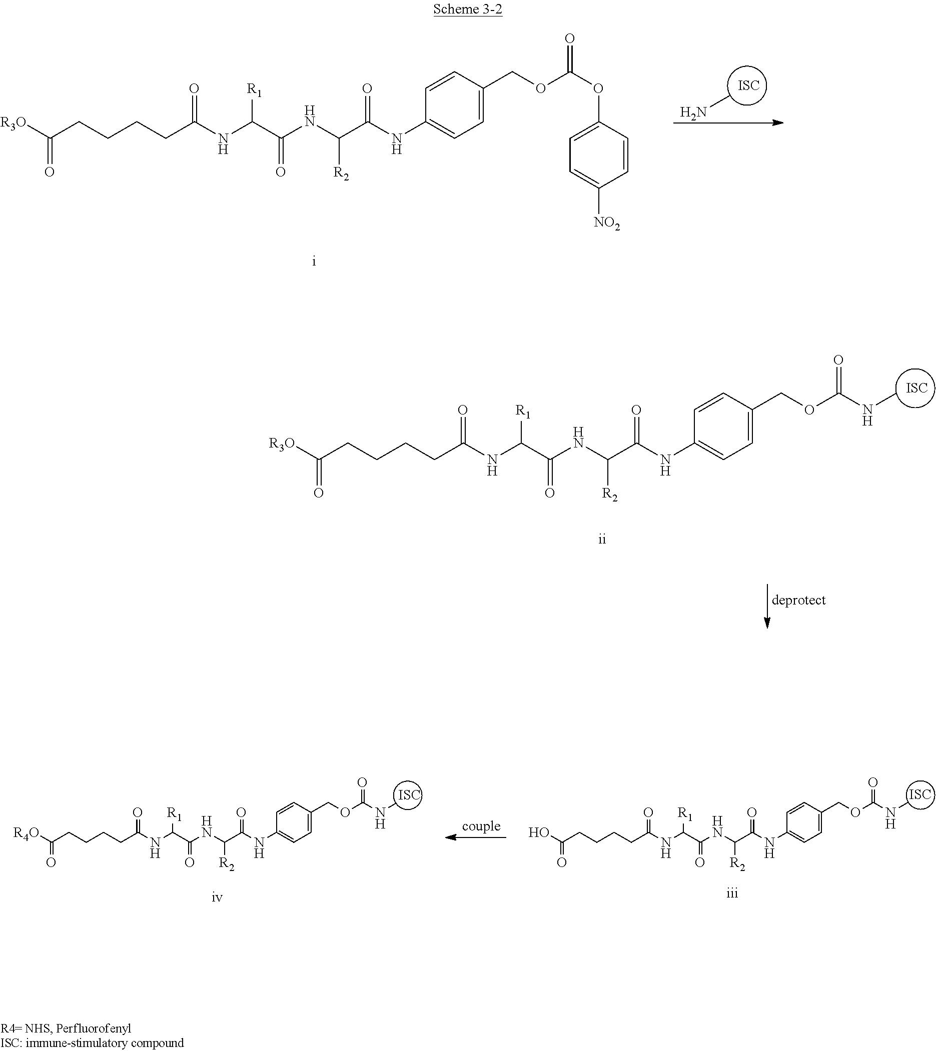

An activated carbonate such as (i) can be reacted with an appropriately substituted amine containing immune-stimulatory compound to afford carbamates (ii) which can be deprotected using standard methods based on the nature of the R³ ester group. The resulting carboxylic acid (iii) can then by coupled with an activating agent such as N-hydroxysuccinimide or pentafluorophenol to provide compounds (iv).

An LP compound can be synthesized as that shown in Scheme 3-3.

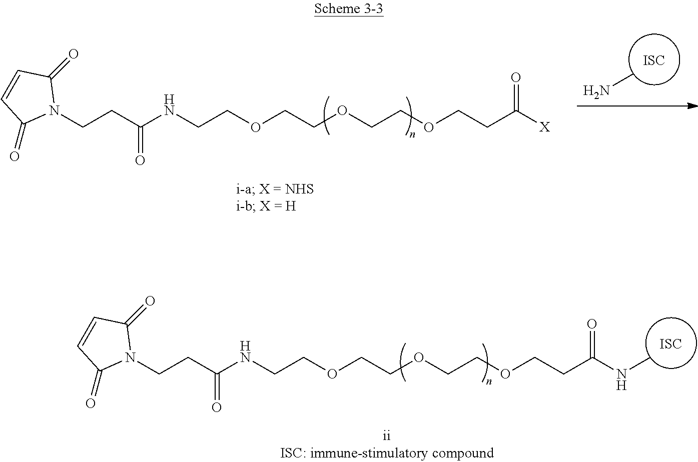

An activated carboxylic ester such as (i-a) can be reacted with an appropriately substituted amine containing immune-modstimulatory compound to afford amides (ii). Alternatively, carboxylic acids of type (i-b) can be coupled to an appropriately substituted amine containing immune-stimulatory compound in the presence of an amide bond forming agent such as dicyclohexycarbodiimde (DCC) to provide the desired LP.

An LP compound can be synthesized by various methods such as that shown in Scheme 3-4.

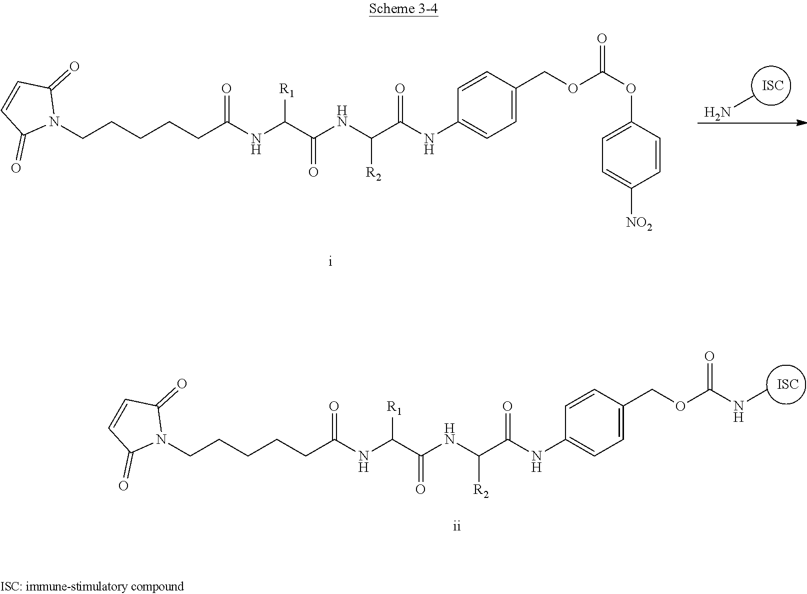

An activated carbonate such as (i) can be reacted with an appropriately substituted amine containing immune-stimulatory compound to afford carbamates (ii) as the target ISC.

An LP compound can also be synthesized as shown in Scheme 3-5.

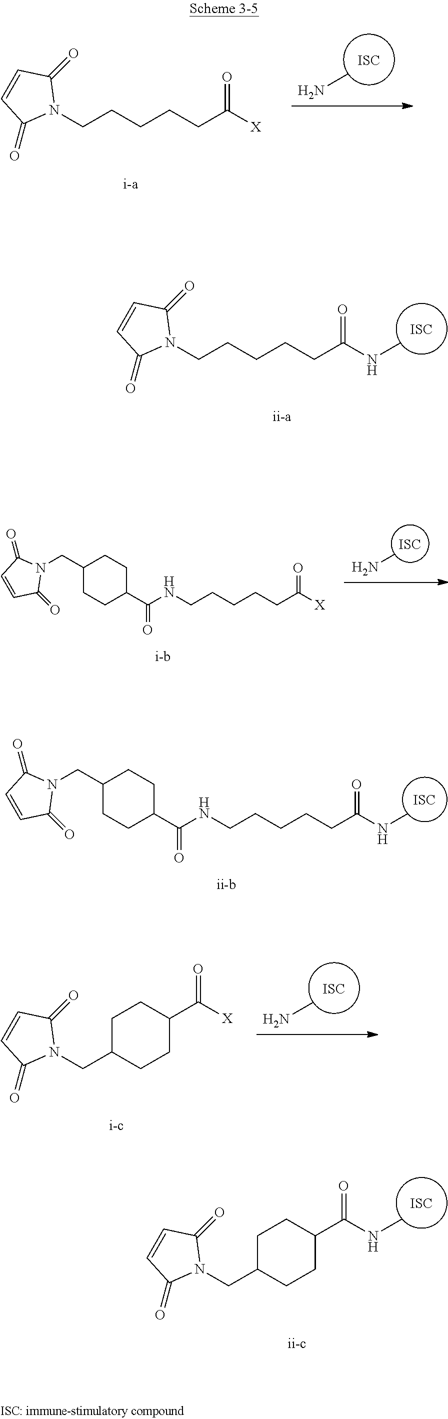

An activated carboxylic acid such as (i-a, i-b, i-c) can be reacted with an appropriately substituted amine containing immune-stimulatory compound to afford amides (ii-a, ii-b, ii-c) as the target linkered payloads (LPs).

### Example 2

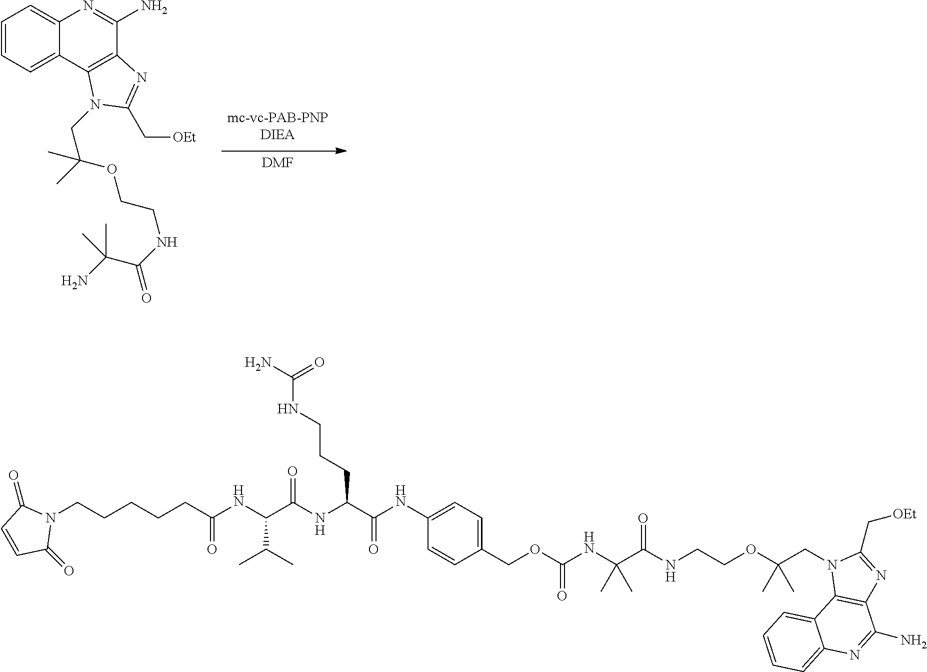

### Compound 2.1: 4-((S)-2-((S)-2-(6-(2,5-dioxo-2,5-dihydro-1H-pyrrol-1-yl)hexanamido)-3-methylbutanamido)-5-ureidopentanamido)benzyl (1-((2-((1-(4-amino-2-(ethoxymethyl)-1H-imidazo[4,5-c]quinolin-1-yl)-2-methylpropan-2-yl)oxy)ethyl)amino)-2-methyl-1-oxopropan-2-yl)carbamate

To a mixture of 2-amino-N-(2-((1-(4-amino-2-(ethoxymethyl)-1H-imidazo[4,5-c]quinolin-1-yl)-2-methylpropan-2-yl)oxy)ethyl)-2-methylpropanamide 1.2 (78.0 mg, 0.18 mmol) and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (0.9 equiv., 117 mg, 0.16 mmol) in DMF (5.0 mL) was added diisopropylethylamine (4.0 equiv., 123 μL, 0.77 mmol). The mixture was stirred at 40° C. for 21h. The crude reaction was purified via preparative RP-HPLC (0→100% AcN in H₂O, 0.1% TFA). Pure fractions were pooled, frozen and dried via lyophilization to give 86 mg (47% yield) of the desired product as an off-white solid.

LCMS (M+H)=1042.2.

1H NMR (DMSO, 400 MHz) δ 13.50 (s, 1H), 9.97 (s, 1H), 8.54 (d, 1H, J=8.4 Hz), 8.12 (d, 1H, J=6.8 Hz), 8.07 (d, 1H, 7.6 Hz), 7.79 (apparent t, 2H, J=7.60 Hz), 7.68 (t, 1H, J=8.0 Hz), 7.57 (d, 2H, J=8.8 Hz), 7.53 (t, 1H, J=7.6 Hz), 7.32-7.17 (m, 4H), 6.98 (s, 2H), 6.98 (d, 1H, J=6.8 Hz), 6.92 (d, 1H, J=6.8 Hz), 6.00 (bs, 1H), 4.87 (s, 3H), 4.35 (quintet, 3H, J=6.8 Hz), 4.18 (dd, 2H, J=8.4, 6.8 Hz), 3.55 (t, 2H, J=7.2 Hz), 3.36 (t, 2H, J=6.8 Hz), 3.20-3.08 (m, 2H), 3.60-2.90 (m, 2H), 2.78-2.89 (m, 2H), 2.17 (quintet, 1H, J=6.4 Hz), 2.13 (quintet, 1H, J=6.4 Hz), 1.94 (septet, 1H, J=6.4 Hz), 1.74-1.63 (m, 1H), 1.63-1.54 (m, 1H), 1.53-1.39 (m, 6H), 1.38-1.27 (m, 2H), 1.21 (s, 6H), 1.22-1.10 (m, 4H), 1.14 (t, 6H, J=6.8 Hz), 0.83 (dd, 6H, J=12.4, 6.8 Hz).

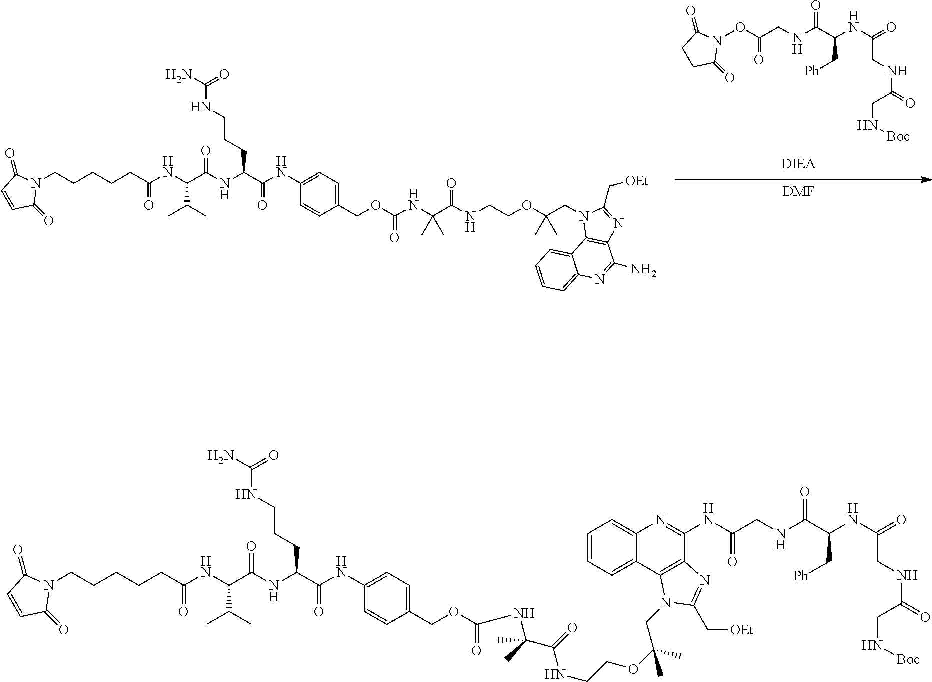

### Compound 2.2: tert-butyl (2-((2-(((S)-1-((2-((1-(1-(4-((S)-2-((S)-2-(6-(2,5-dioxo-2,5-dihydro-1H-pyrrol-1-yl)hexanamido)-3-methylbutanamido)-5-ureidopentanamido)phenyl)-5,5,11,11-tetramethyl-3,6-dioxo-2,10-dioxa-4,7-diazadodecan-12-yl)-2-(ethoxymethyl)-1H-imidazo[4,5-c]quinolin-4-yl)amino)-2-oxoethyl)amino)-1-oxo-3-phenylpropan-2-yl)amino)-2-oxoethyl)amino)-2-oxoethyl)carbamate

To a mixture of 4-((S)-2-((S)-2-(6-(2,5-dioxo-2,5-dihydro-1H-pyrrol-1-yl)hexanamido)-3-methylbutanamido)-5-ureidopentanamido)benzyl (1-((2-((1-(4-amino-2-(ethoxymethyl)-1H-imidazo[4,5-c]quinolin-1-yl)-2-methylpropan-2-yl)oxy)ethyl)amino)-2-methyl-1-oxopropan-2-yl)carbamate 2.1 (21.0 mg, 0.02 mmol) and 2,5-dioxopyrrolidin-1-yl (tert-butoxycarbonyl)glycylglycyl-L-phenylalanylglycinate (2.0 equiv., 21.0 mg, 0.04 mmol) in DMF (2.5 mL) was added diisopropylethylamine (4.0 equiv., 15 μL, 0.08 mmol). The mixture was stirred at 40° C. for 17h. The crude reaction was purified via preparative RP-HPLC (0→70% AcN in H₂O, 0.1% TFA). Pure fractions were pooled, frozen and dried via lyophilization to give 4.2 mg (14% yield) of the desired product as an off-white solid. LCMS (M+H)=1460.2.

Other compounds of the same class of described above may be prepared in a manner similar to that described in Example 2 above using the appropriate reagents.

TABLE 2
Compounds 2.1-2.20

| Compound | Structure | Spectroscopic Data |
|---|---|---|
| 2.1 | 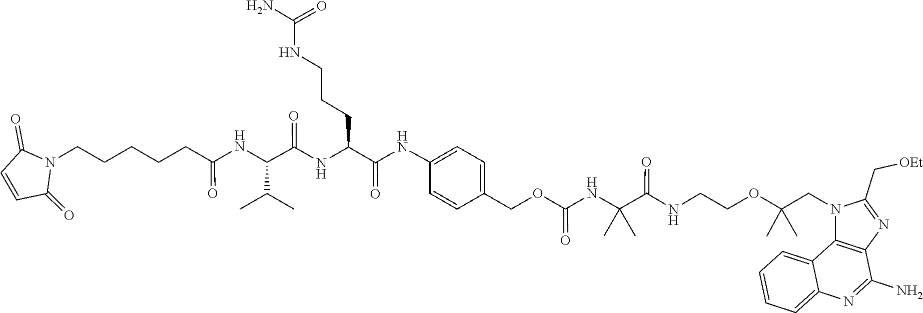 [SMILES: CCOCc1nc2c(N)nc3ccccc3c2n1CC(C)(C)OCCNC(=O)C(C)(C)NC(=O)OCc1ccc(NC(=O)[C@H](CCCNC(N)=O)NC(=O)[C@@H](NC(=O)CCCCCN2C(=O)C=CC2=O)C(C)C)cc1] | 1H NMR (DMSO, 400 MHz) δ 13.50 (s, 1H), 9.97 (s, 1H), 8.54 (d, 1H, J = 8.4 Hz), 8.12 (d, 1H, J = 6.8 Hz), 8.07 (d, 1H, 7.6 Hz), 7.79 (apparent t, 2H, J = 7.60 Hz), 7.68 (t, 1H, J = 8.0 Hz), 7.57 (d, 2H, J = 8.8 Hz), 7.53 (t, 1H, J = 7.6 Hz), 7.32- 7.17 (m, 4H), 6.98 (s, 2H), 6.98 (d, 1H, J = 6.8 Hz), 6.92 (d, 1H, J = 6.8 Hz), 6.00 (bs, 1H), 4.87 (s, 3H), 4.35 (quintet, 3H, J = 6.8 Hz), 4.18 (dd, 2H, J = 8.4, 6.8 Hz), 3.55 (t, 2H, J = 7.2 Hz), 3.36 (t, 2H, J = 6.8 Hz), 3.20-3.08 (m, 2H), 3.60-2.90 (m, 2H), 2.78-2.89 (m, 2H), 2.17 (quintet, 1H, J = 6.4 Hz), 2.13 (quintet, 1H, J = 6.4 Hz), 1.94 (septet, 1H, J = 6.4 Hz), 1.74- 1.63 (m, 1H), 1.63-1.54 (m, H), 1.53-1.39 (m, 6H), 1.38-1.27 (m, 2H), 1.21 (s, 6H), 1.22-1.10 (m, 4H), 1.14 (t, 6H, J = 6.8 Hz), 0.83 (dd, 6H, J = 12.4, 6.8 Hz). |
| | | LCMS (M + H) = 1042.2. |
| | 4-((S)-2-((S)-2-(6-(2,5-dioxo-2,5-dihydro-1H-pyrrol-1-yl)hexanamido)-3-methylbutanamido)-5- | |
| | ureidopentanamido)benzyl(1-((2-((1-(4-amino-2-(ethoxymethyl)-1H-imidazo[4,5-c]quinolin-1- | |
| | yl)-2-methylpropan-2-yl)oxy)ethyl)amino)-2-methyl-1-oxopropan-2-yl)carbamate | |
| 2.2 | 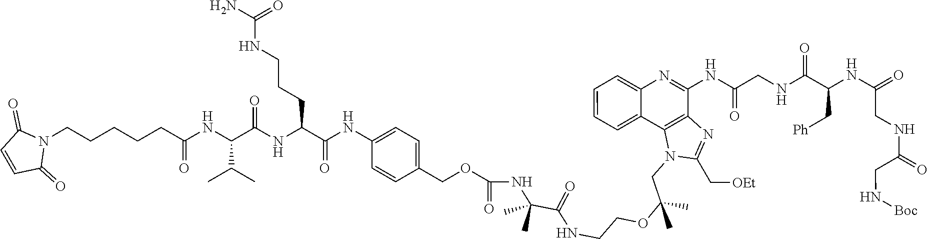 | LCMS (M + H) = 1460.2 |
| | tert-butyl (2-((2-(((S)-1-((2-((1-(1-(4-((S)-2-((S)-2-(6-(2,5-dioxo-2,5-dihydro-1H-pyrrol-1-yl)hexanamido)-3-methylbutanamido)- | |
| | 5-ureidopenlanamido)phenyl)-5,5,11,11-tetramethyl-3,6-dioxo-2,10-dioxa-4,7-diazadodecan-12-yl)-2-(ethoxymethyl)-1H- | |
| | imidazo[4,5-c]quinolin-4-yl)amino)-2-oxoethyl)amino)-1-oxo-3-phenylpropan-2-yl)amino)-2-oxoethyl)amino)-2-oxoethyl)carbamate | |
| 2.3 | 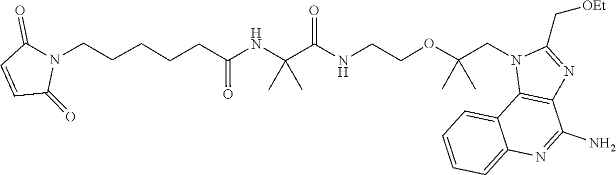 [SMILES: CCOCc1nc2c(N)nc3ccccc3c2n1CC(C)(C)OCCNC(=O)C(C)(C)NC(=O)CCCCCN1C(=O)C=CC1=O] | LCMS (M + H) = 636.8 |
| | N-(1-((2-((1-(4-amino-2-(ethoxymethyl)-1H-imidazo[4,5-c]quinolin-1-yl)-2-methylpropan-2- | |
| | yl)oxy)ethyl)amino)-2-methyl-1-oxopropan-2-yl)-6-(2,5-dioxo-2,5-dihydro-1H-pyrrol-1-yl)hexanamide | |
| 2.4 | 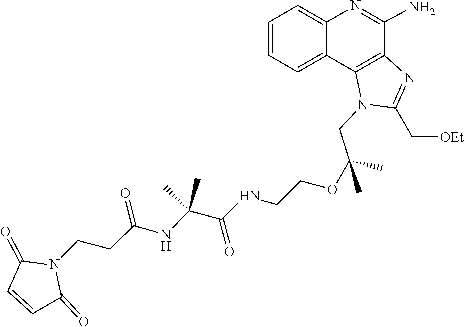 | LCMS (M + H) = 594.7 |
| | N-(2-((1-(4-amino-2-(ethoxymethyl)-1H-imidazo[4,5-c]quinolin-1-yl)-2-methylpropan-2-yl)oxy)ethyl)-2- | |
| | (3-(2,5-dioxo-2,5-dihydro-1H-pyrrol-1-yl)propanamido)-2-methylpropanamide | |
| 2.5 | 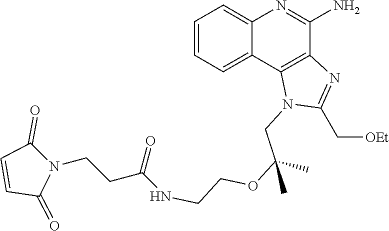 | LCMS (M + H) = 509.6 |
| | N-(2-((1-(4-amino-2-(ethoxymethyl)-1H-imidazo[4,5-c]quinolin-1-yl)-2- | |
| | methylpropan-2-yl)oxy)ethyl)-3-(2,5-dioxo-2,5-dihydro-1H-pyrrol-1- | |
| | yl)propanamide | |
| 2.6 | 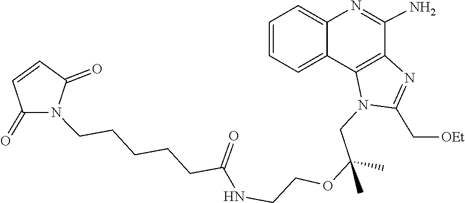 | LCMS (M + H) = 551.7 |
| | N-2-((1-(4-amino-2-(ethoxymethyl)-1H-imidazo[4,5-c]quinolin-1-yl)-2-methylpropan- | |
| | 2-yl)oxy)ethyl)-6-(2,5-dioxo-2,5-dihydro-1H-pyrrol-1-yl)hexanamide | |
| 2.7 | 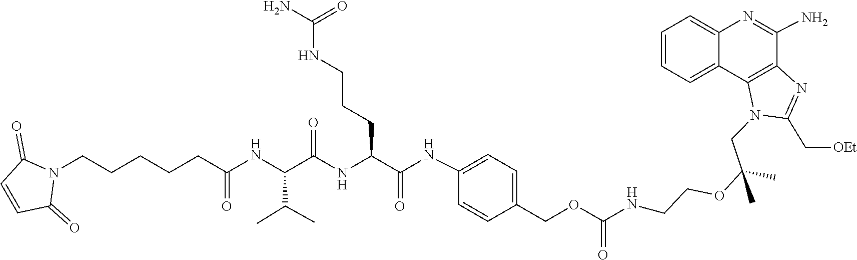 | LCMS (M + H) = 957.1 |
| | 4-((S)-2-((S)-2-(6-(2,5-dioxo-2,5-dihydro-1H-pyrrol-1-yl)hexanamido)-3-methylbutanamido)-5-ureidopentanamido)benzyl | |
| | (2-((1-(4-amino-2-(ethoxymethyl)-1H-imidazo[4,5-c]quinolin-1-yl)-2-methylpropan-2-yl)oxy)ethyl)carbamate | |
| 2.8 | 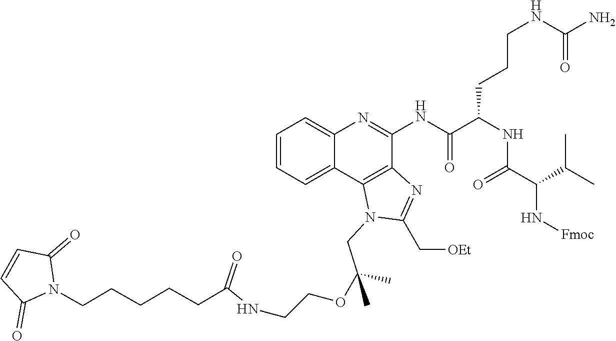 | LCMS (M + H) = 1030.2 |
| | (9H-fluoren-9-yl)methyl ((S)-1-(((S)-1-((1-(2-(2-(6-(2,5-dioxo-2,5-dihydro-1H-pyrrol-1-yl)hexanamido)ethoxy)- | |
| | 2-methylpropyl)-2-(ethoxymethyl)-1H-imidazo[4,5-c]quinolin-4-yl)amino)-1-oxo-5-ureidopentan-2-yl)amino)-3- | |
| | methyl-1-oxobutan-2-yl)carbamate | |
| 2.9 | 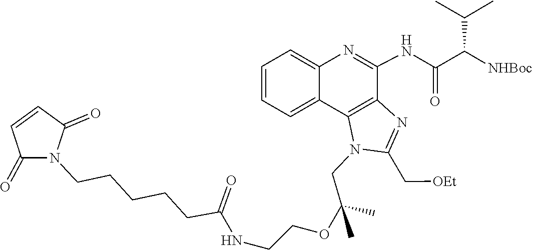 | LCMS (M + H) = 750.9 |
| | tert-butyl (S)-(1-((1-(2-(2-(6-(2,5-dioxo-2,5-dihydro-1H-pyrrol-1-yl)hexanamido)ethoxy)- | |
| | 2-methylpropyl)-2-(ethoxymethyl)-1H-imidazo[4,5-c]quinolin-4-yl)amino)-3-methyl-1- | |
| | oxobutan-2-yl)carbamate | |
| 2.10 | 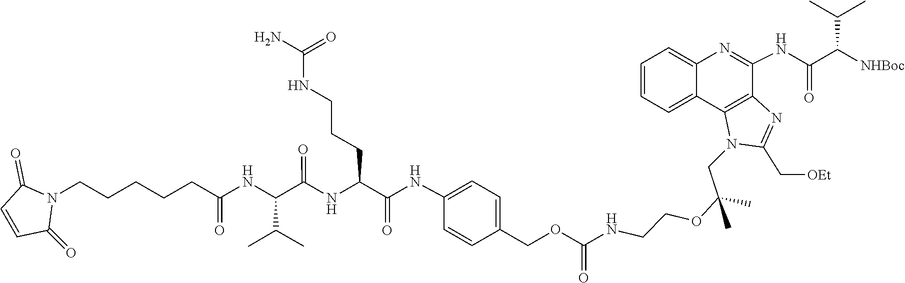 | LCMS (M + H) = 1136.6 |
| | tert-butyl ((S)-1-((1-(2-(2-((((4-((S)-2-((S)-2-(6-(2,5-dioxo-2,5-dihydro-1H-pyrrol-1-yl)hexanamido)-3-methylbutanamido)-5- | |
| | ureidopentanamido)benzyl)oxy)carbonyl)amino)ethoxy)-2-methylpropyl)-2-(ethoxymethyl)-1H-imidazo[4,5- | |
| | c]quinolin-4-yl)amino)-3-methyl-1-oxobutan-2-yl)carbamate | |
| 2.11 | 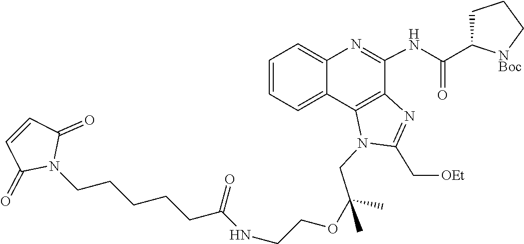 | LCMS (M + H) = 748.9 |
| | tert-butyl(S)-2-((1-(2-(2-(6-(2,5-dioxo-2,5-dihydro-1H-pyrrol-1-yl)hexanamido)ethoxy)-2- | |
| | methylpropyl)-2-(ethoxymethyl)-1H-imidazo[4,5-c]quinolin-4-yl)carbamoyl)pyrrolidine-1-carboxylate | |
| 2.12 | 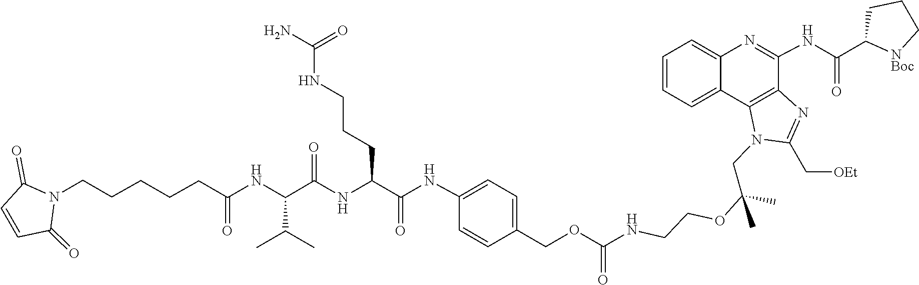 | LCMS (M + H) = 1154.4 |
| | tert-butyl (S)-2-((1-(2-(2-((((4-((S)-2-((S)-2-(6-(2,5-dioxo-2,5-dihydro-1H-pyrrol-1-yl)hexanamido)-3-methylbutanamido)-5- | |
| | ureidopentanamido)benzyl)oxy)carbonyl)amino)ethoxy)-2-methylpropyl)-2-(ethoxymethyl)-1H-imidazo[4,5-c]quinolin-4- | |
| | yl)carbamoyl)pyrrolidine-1-carboxylate | |
| 2.13 | 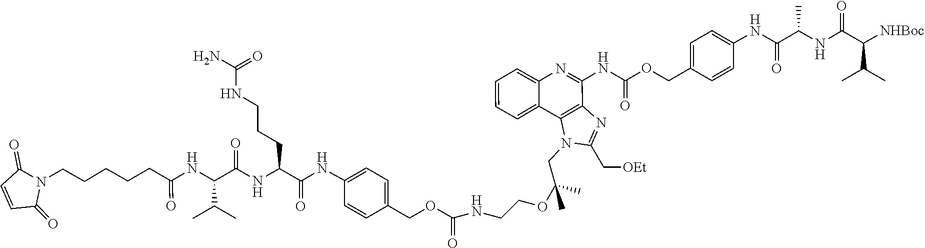 | LCMS (M + H) = 1376.6 |
| | 4-((S)-2-((S)-2-((tert-butoxycarbonyl)amino)-3-methylbutanamido)propanamido)benzyl(1-(2-(2-((((4-((S)-2- | |
| | ((S)-2-(6-(2,5-dioxo-2,5-dihydro-1H-pyrrol-1-yl)hexanamido)-3-methylbutanamido)-5- | |
| | ureidopentanamido)benzyl)oxy)carbonyl)amino)ethoxy)-2-methylpropyl)-2-(ethoxymethyl)-1H-imidazo[4,5- | |
| | c]quinolin-4-yl)carbamate | |
| 2.14 | 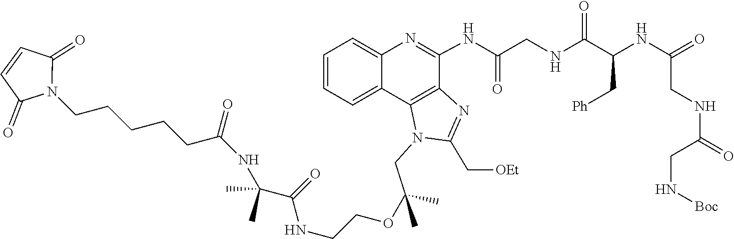 | LCMS (M + H) = 1055.2 |
| | tert-butyl (S)-(2-((2-((1-((2-((1-(2-(2-(2-(6-(2,5-dioxo-2,5-dihydro-1H-pyrrol-1-yl)hexanamido)-2-methylpropanamido)ethoxy)- | |
| | 2-methylpropyl)-2-(ethoxymethyl)-1H-imidazo[4,5-c]quinolin-4-yl)amino)-2-oxoethyl)amino)-1-oxo-3-phenylpropan-2-yl)amino)- | |
| | 2-oxoethyl)amino)-2-oxoethyl)carbamate | |
| 2.15 | 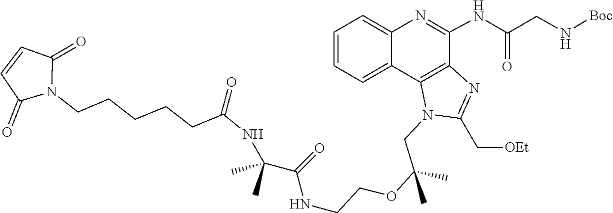 | LCMS (M + H) = 793.9 |
| | tert-butyl (2-((1-(2-(2-(2-(6-(2,5-dioxo-2,5-dihydro-1H-pyrrol-1-yl)hexanamido)-2-methylpropanamido)ethoxy)-2- | |
| | methylpropyl)-2-(ethoxymethyl)-1H-imidazo[4,5-c]quinolin-4-yl)amino)-2-oxoethyl)carbamate | |
| 2.16 | 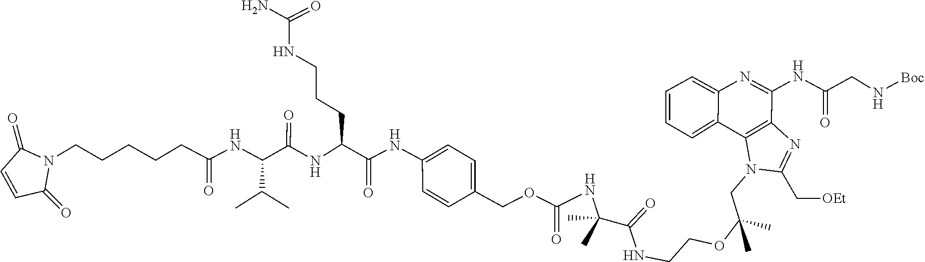 | LCMS (M + H) = 1194.4 |
| | 4-((S)-2-((S)-2-(6-(2,5-dioxo-2,5-dihydro-1H-pyrrol-1-yl)hexanamido)-3-methylbutanamido)-5-ureidopentanamido)benzyl | |
| | (1-((2-((1-(4-(2-((tert-butoxycarbonyl)amino)acetamido)-2-(ethoxymethyl)-1H-imidazo[4,5-c]quinolin-1-yl)-2-methylpropan-2- | |
| | yl)oxy)ethyl)amino)-2-methyl-1-oxopropan-2-yl)carbamate | |
| 2.17 | 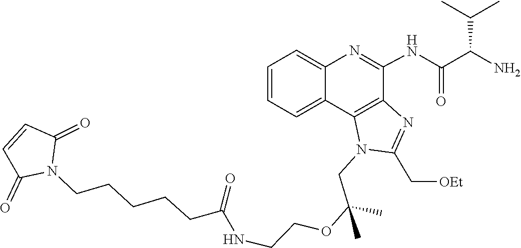 | LCMS (M + H) = 650.8 |
| | (S)-N-(2-((1-(4-(2-amino-3-methylbutanamido)-2-(ethoxymethyl)-l H-imidazo[4,5-c]quinolin-1-yl)-2- | |
| | methylpropan-2-yl)oxy)ethyl)-6-(2,5-dioxo-2,5-dihydro-1H-pyrrol-1-yl)hexanamide | |
| 2.18 | 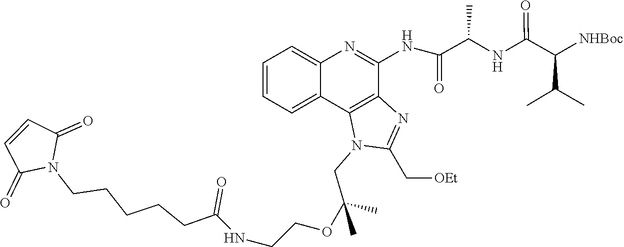 | LCMS (M + Na) = 1248.4 LCMS (M + H − Boc) = 721.0 |
| | tert-butyl ((S)-1-(((S)-1-((1-(2-(2-(6-(2,5-dioxo-2,5-dihydro-1H-pyrrol-1-yl)hexanamido)ethoxy)-2-methylpropyl)-2- | |
| | (ethoxymethyl)-1H-imidazo[4,5-c]quinolin-4-yl)amino)-1-oxopropan-2-yl)amino)-3-methyl-1-oxobutan-2-yl)carbamate | |
| 2.19 | 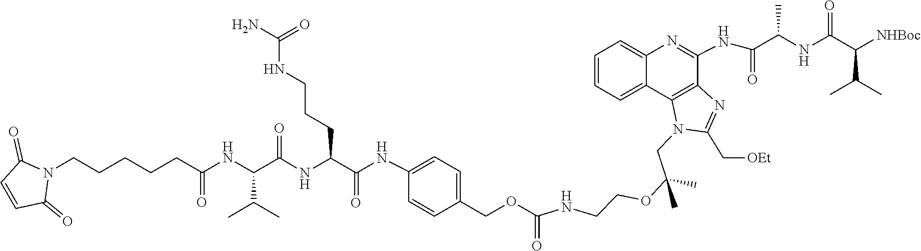 | LCMS (M + Na) = 843.0 LCMS (M + H − Boc) = 1126.4 |
| | tert-butyl ((S)-1-(((S)-1-((1-(2-(2-((((4-((S)-2-((S)-2-(6-(2,5-dioxo-2,5-dihydro-1H-pyrrol-1-yl)hexanamido)-3-methylbutanamido)-5- | |
| | ureidopentanamido)benzyl)oxy)carbonyl)amino)ethoxy)-2-methylpropyl)-2-(ethoxymethyl)-1H-imidazo[4,5-c]quinolin-4-yl)amino)- | |
| | 1-oxopropan-2-yl)amino)-3-methyl-1-oxobutan-2-yl)carbamate | |
| 2.20 | 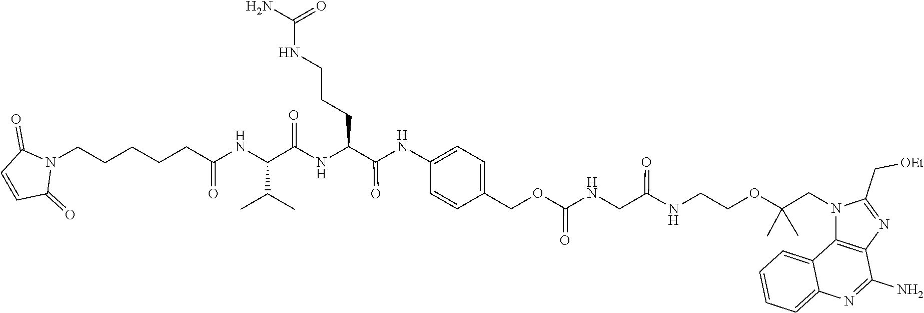 [SMILES: CCOCc1nc2c(N)nc3ccccc3c2n1CC(C)(C)OCCNC(=O)CNC(=O)OCc1ccc(NC(=O)[C@H](CCCNC(N)=O)NC(=O)[C@@H](NC(=O)CCCCCN2C(=O)C=CC2=O)C(C)C)cc1] | LCMS (M + H) = 1014.2. |
| | 4-((S)-2-((S)-2-(6-(2,5-dioxo-2,5-dihydro-1H-pyrrol-1-yl)hexanamido)-3-methylbutanamido)-5-ureidopentanamido)benzyl | |
| | (2-((2-((1-(4-amino-2-(ethoxymethyl)-1H-imidazo[4,5-c]quinolin-1-yl)-2-methylpropan-2-yl)oxy)ethyl)amino)-2-oxoethyl)carbamate | |

### Example 3

### Synthesis of Antibody-Drug Conjugates

### Protocol for the Preparation of Antibody Conjugates Via Partial Reduction of Interchain-Disulfides

The mAb (3-8 mg/mL in PBS) was exchanged into HEPES (100 mM, pH 7.0, 1 mM DTPA) via molecular weight cut-off centrifugal filtration (Millipore, 30 kDa). The resultant mAb solution was transferred to a tared 50 mL conical tube. The mAb concentration was determined to be 3-8 mg/mL by A₂₈₀. To the mAb solution was added TCEP (2.0-4.0 equiv., 1 mM stock) at room temperature and the resultant mixture was incubated at 37° C. for 30-90 min., with gentle shaking. Upon being cooled to room temperature, a stir bar was added to the reaction tube. With stirring, DMA (5-15% v/v) was added dropwise to the reaction mixture. Next, the linker-payload (5.0-13.0 equiv., 10 mM DMA) was added dropwise. The resultant reaction mixture was allowed to stir at ambient temperature for 30-60 minutes, at which point N-ethyl maleimide (3.0 equiv., 100 mM DMA) was added. After an additional 15 minutes of stirring, cysteine (6.0-11.0 equiv., 50 mM HEPES) was added. The crude ADC was then exchanged into PBS and purified by preparative SEC (e.g. HiLoad 26/600, Superdex 200 pg) using PBS as the mobile phase. The pure fractions were concentrated via molecular weight cut-off centrifugal filtration (Millipore, 30 kDa), sterile filtered and transferred to 15 mL conical tubes. Following characterization, the ADC was characterized by analytical SEC, analytical HIC, analytical reversed phase, LCMS, UV-VIS and Endotoxin.

### Example 4

### General Procedure for the Determination of the Drug-Antibody-Ratios

### Hydrophobic Interaction Chromatography

10 μL of a 6 mg/mL solution of the conjugate was injected into an HPLC system set-up with a TOSOH TSKgel Butyl-NPR TM hydrophobic interaction chromatography (HIC) column (2.5 μM particle size, 4.6 mm×35 mm) attached. Then, over the course of 18 minutes, a method was run in which the mobile phase gradient ran from 100% mobile phase A to 100% mobile phase B over the course of 12 minutes, followed by a six-minute re-equilibration at 100% mobile phase A. The flow rate was 0.8 mL/min and the detector was set at 280 nM. Mobile phase A was 1.5 M ammonium sulfate, 25 mM sodium phosphate (pH 7). Mobile phase B was 25% isopropanol in 25 mM sodium phosphate (pH 7). Post-run, the chromatogram was integrated and the molar ratio was determined by summing the weighted peak area.

### Mass Spectrometry

One microgram of conjugate was injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact antibody construct conjugate was calculated by the software, which used top peak height at 25% for the calculation. This data is then imported into another program to calculate the molar ratio of the conjugate such as Agilent molar ratio calculator.

### Example 5

TABLE 3
Conjugate Characterization

| | | | Concentration in |
|---|---|---|---|
| | | | PBS (by A280) |
| Conjugate | DAR by HIC | % Monomer by SEC | (mg/mL) |
| anti-Her2-mIgG2a- | 4.10 | >97% | 8.04 |
| Compound 2.1 | | | |
| anti-Her2-mIgG2a- | 3.85 | >97% | 4.92 |
| Compound 2.3 | | | |
| anti-Her2-mIgG2a- | 3.85 | >97% | 5.49 |
| Compound 2.4 | | | |
| anti-Her2-mIgG2a- | 3.80 | >97% | 4.60 |
| Compound 2.5 | | | |
| anti-Her2-mIgG2a- | 3.80 | >97% | 5.33 |
| Compound 2.6 | | | |
| anti-Her2-mIgG2a- | 3.83 | >97% | 3.62 |
| Compound 2.7 | | | |
| anti-Her2-mIgG2a- | 3.70 | >97% | 4.60 |
| Compound 2.20 | | | |

The anti-Her2-mIgG2a in Example 5 is a mouse chimeric antibody comprising pertuzumab CDRs.

### Example 6

### Murine TNFα Production by Murine Macrophages was Induced by TLR7 Small Molecules

Murine macrophages were derived from bone marrow cells from a Balb/c mouse. Briefly, murine bone marrow cells were isolated and resuspended in cDMEM media (high glucose DMEM (HyClone) supplemented with 10% fetal bovine serum (HyClone), 1 mM Sodium Pyruvate (HyClone), 2 mM L-glutamine (Gibco), 10 mM HEPES (HyClone), 1×NEAA (Gibco), 50 U/mL penicillin, 50 U/mL streptomycin (HyClone)) and seeded at 5e5 cells/mL in the presence of 20 ng/mL murine M-CSF (Peprotech) and incubated at 37° C., 5% CO₂ for 7 days to differentiate into murine macrophages.

### General Procedure for In Vitro Small Molecule Screening

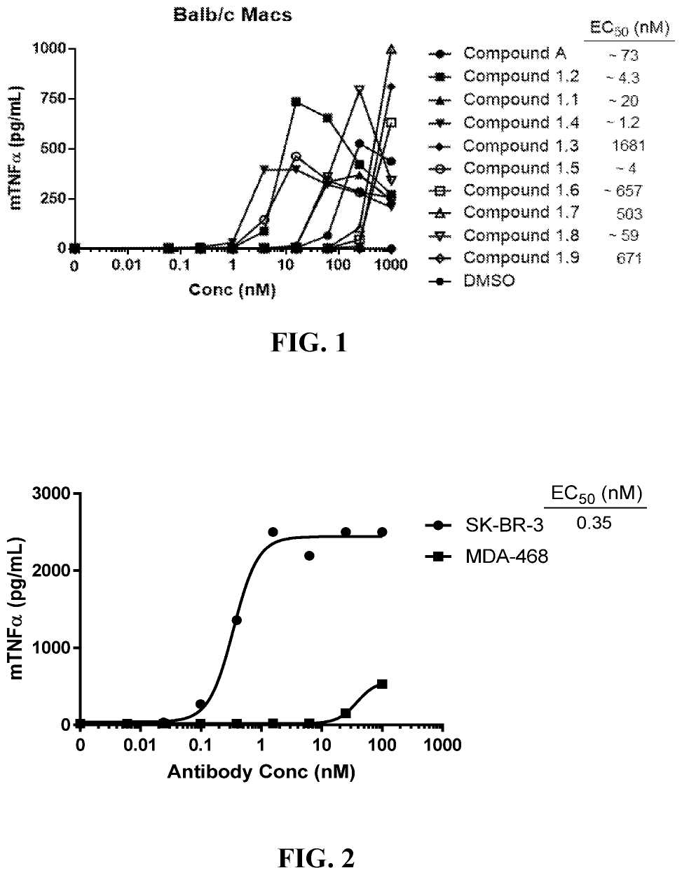

This example shows that TLR7 small molecules can increase production of a pro-inflammatory cytokine, murine TNFα, from bone marrow-derived murine macrophages. After differentiation, bone marrow-derived murine macrophages were plated in 96-well flat bottom microtiter plates (80,000/well) along with titrating concentrations of TLR7 small molecules ranging from 1000 to 0.06 nM in cRPMI media (RPMI-1640 (HyClone) supplemented with 10% fetal bovine serum (HyClone), 1 mM Sodium Pyruvate (HyClone), 2 mM L-glutamine (Gibco), 10 mM HEPES (HyClone), 1×NEAA (Gibco), 50 U/mL penicillin, 50 U/mL streptomycin (HyClone)). After overnight culture, supernatants were harvested and murine TNFα levels were determined by ELISA (BioLegend). Table 4. FIG. 1 shows in vitro TLR7 small molecule screening. Data was analyzed using GraphPad Prism 7.01 software (GraphPad Software) and EC₅₀ values calculated using non-linear regression. The TLR7 small molecules were active, stimulating production of murine TNFα in a dose-dependent manner from the murine macrophages. Compound A is represented by the formula:

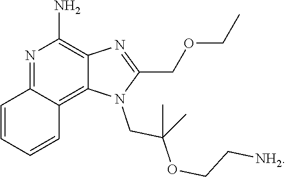 [SMILES: CCOCc1nc2c(N)nc3ccccc3c2n1CC(C)(C)OCCN]

TABLE 4
In vitro TLR7 small molecule screening

| Compound | EC₅₀ (nM) |
|---|---|
| Compound A | 73 |
| Compound 1.1 | 20 |
| Compound 1.2 | 4.3 |
| Compound 1.3 | 1681 |
| Compound 1.4 | 1.2 |
| Compound 1.5 | 4 |
| Compound 1.6 | 657 |
| Compound 1.7 | 503 |
| Compound 1.8 | 59 |
| Compound 1.9 | 671 |

### Example 7

### Murine TNFα Production by Murine Macrophages was Induced by Immune Stimulatory Conjugates

### General Procedure for Immune Stimulatory Conjugate Screening

This example shows that immune-stimulatory conjugates can increase production of a pro-inflammatory cytokine, murine TNFα, from bone marrow-derived murine macrophages in the presence of antigen expressing tumor cells.

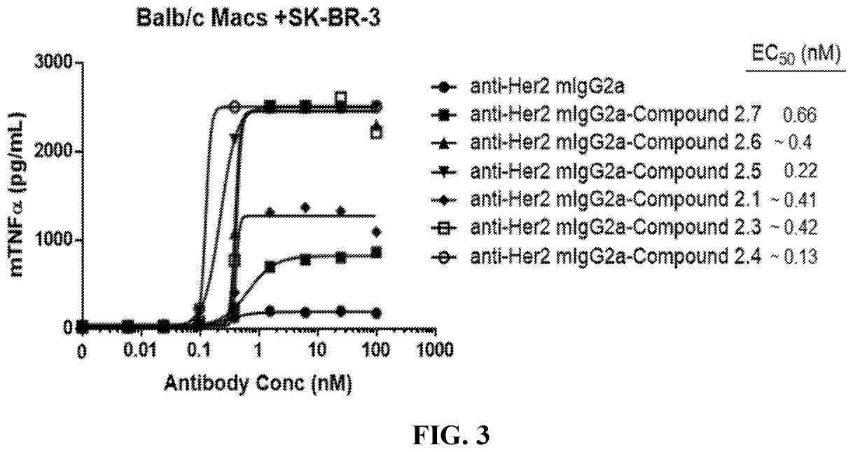

Murine bone marrow cells were differentiated into macrophages as described above. After differentiation, bone marrow-derived murine macrophages were plated in 96-well flat bottom microtiter plates (80,000/well) in cRPMI assay media. Antigen-expressing or antigen non-expressing tumor cells were then added (40,000/well) along with titrating concentrations of conjugates or control antibodies ranging from 100 to 0.006 nM in cRPMI media. After overnight culture, supernatants were harvested and murine TNFα levels were determined by ELISA (BioLegend). Table 5. Data was analyzed using GraphPad Prism 7.01 software (GraphPad Software) and EC₅₀ values calculated using non-linear regression. All the conjugates were active, stimulating production of murine TNFα in a dose-dependent manner from the murine macrophages in the presence of Her2 antigen expressing SK-BR-3 cells. In contrast, the conjugates did not stimulate production of murine TNFα from the murine macrophages in the absence of Her2 antigen on non-expressing MDA-MB-468 cells. FIG. 2 and FIG. 3 show in vitro Her2/TLR7 Immune Stimulatory Conjugate screening.

TABLE 5
In vitro Her2-TLR7Immune Stimulatory Conjugate screening

| Conjugate | EC₅₀ (nM) |
|---|---|
| Anti-Her2-mIgG2a-Compound 2.1 | 0.35 nM |
| Anti-Her2-mIgG2a-Compound 2.20 | n.d. |

### Example 8

### Anti-Her2-mIgG2a-Compound 2.1 Conjugate Slows Tumor Growth, Increases Survival in Heterotopic Mouse Model of Colon Carcinoma

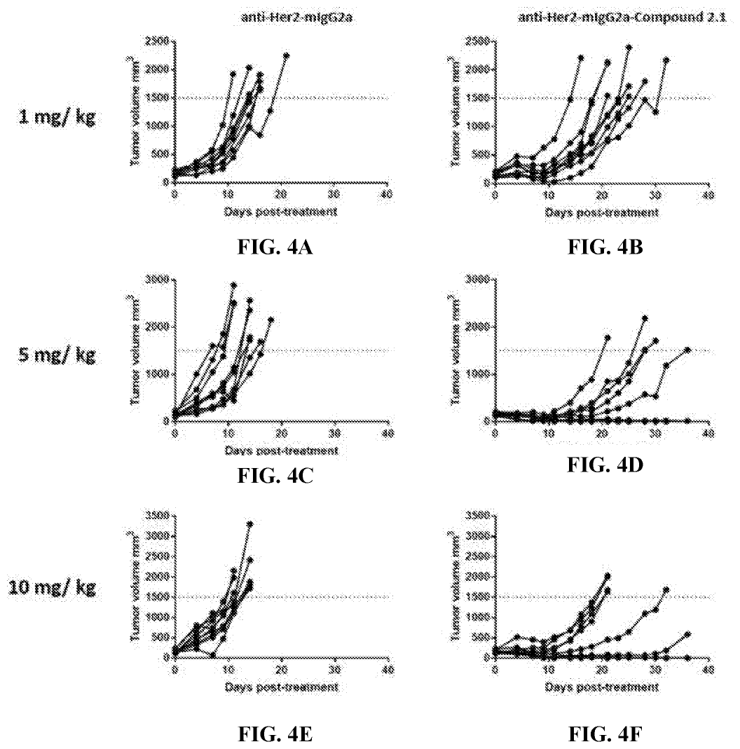

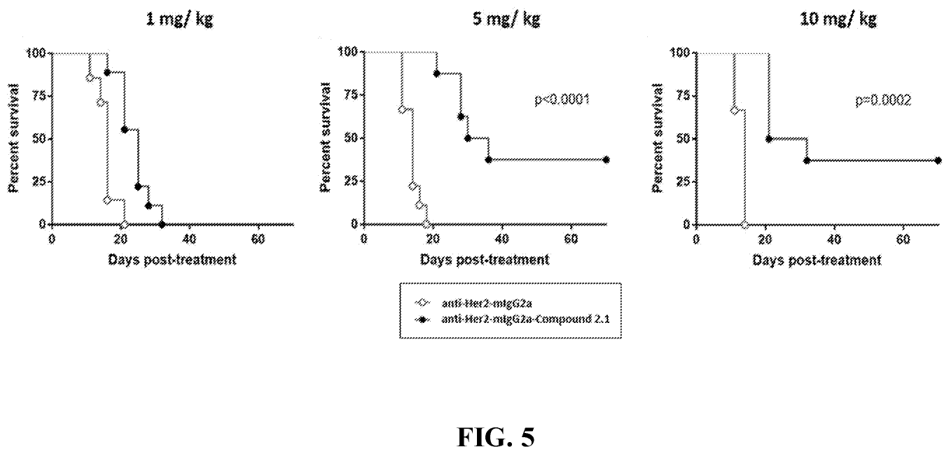

A sterile suspension of 5×10⁵ CT26 colon carcinoma cells expressing human Her2 were injected subcutaneously in a volume of 0.1 ml PBS into the right flank of female Balb/c mice. When tumors reached a volume of 200 mm³, mice were treated with anti-Her2 or anti-Her2-conjugated to a TLR7 agonist (anti-Her2-mIgG2a-Compound 2.1) at 1, 5, 10 mg/kg, 3×per week for 1 week. Tumors volumes were monitored every other day for the duration of the study and mice were euthanized when tumors reached a volume of 1500 mm³ as determined by the formula VOL=(L×W×H×0.5). It was demonstrated that mice treated with anti-Her2-mIgG2a-Compound 2.1 at 5 and 10 mg/kg had reduced tumor volume (FIG. 4A-FIG. 4F) and improved survival (FIG. 5) when compared to those treated with anti-Her2 alone. FIG. 4A-FIG. 4F show treatment with anti-Her2-TLR7 agonist conjugate inhibits tumor growth in CT26-Her2 bearing mice. Tumor volume measurements for mice treated with anti-Her2 (A, C, E) or anti-Her2 conjugated to TLR7 agonist Compound 2.1. N=8 mice per group. FIG. 5 shows treatment with anti-Her2-TLR7 agonist conjugate improves survival of CT26-Her2 bearing mice. Kaplan-Meier curves for mice treated with anti-Her2 or anti-Her2-Compound 2.1 conjugate at 1, 5, 10 mg/kg. N=8 mice per group.

### Example 9

### Treatment of HER2+ EMT6 Syngeneic Tumor Bearing Mice with an Anti-HER2 TLR7 Conjugate Lowers Tumor Growth and Improves Survival

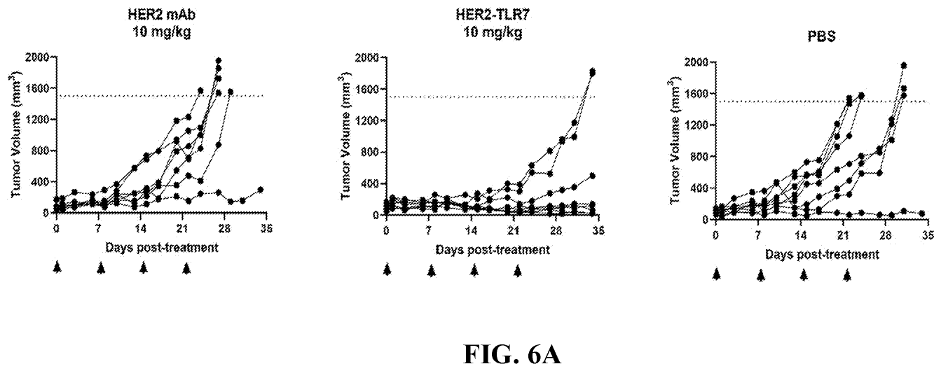

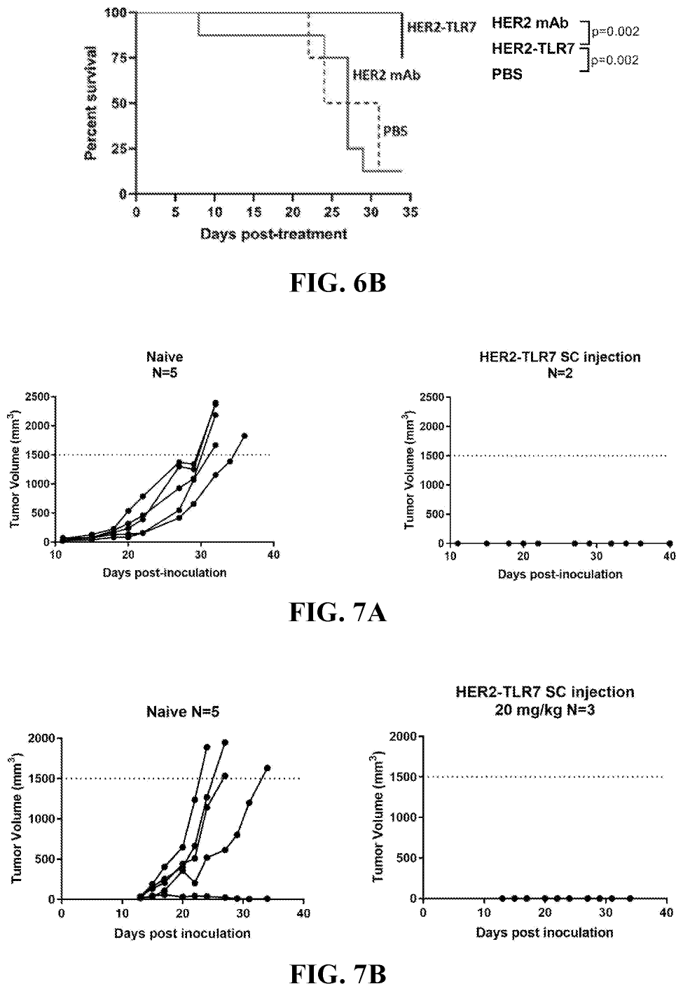

The ability of a TLR7 antibody conjugate to alter tumor cell growth in mouse syngeneic tumor was assessed as follows. Six to seven-week-old Balb/cJ mice were inoculated subcutaneously (SC) in the mammary fat pad with 1×10⁵ HER2+ EMT6 cells. Six days later, tumors were measured with calipers and volume was calculated using the formula volume=((minimum length)²×(maximum length))/2. Mice with tumor volumes ranging from 44.25 to 175.71 mm³ mice were organized into 3 groups of 10 with average tumor size 97.14 mm³. Mice were administered anti-HER2 mAb (mIgG2a) at 10 mg/kg, anti-HER2-mIgG2a-Compound 2.1 of Table 3 (anti-HER2-TLR7 conjugate) at 10 mg/kg, or PBS, SC, once weekly for 4 weeks. Tumor volumes were measured 3 times per week. Mice were euthanized when volumes reached 1500 mm³ or if the tumors metastasized. The study was terminated approximately 5 weeks after the first dose (day 34). Volumes and survival were plotted using GraphPad Prism. Survival curves were analyzed using the Log rank (Mantel-Cox) test. p<0.05 was considered statistically significant. The cohort treated with the anti-HER2-TLR7 conjugate showed slowed tumor growth (FIG. 6A) and a significant survival advantage (FIG. 6B) compared to controls.

### Example 10

### Mice that have Cleared HER2pos CT26 Tumors in Response to Anti-HER2-TLR7 Conjugate Reject HER2pos CT26 Tumors Upon Re-Challenge

These studies were designed to test the durability of the anti-tumor responses in mice treated with anti-HER2-TLR7 conjugate. Mice inoculated with HER2 positive CT26 colon carcinoma cells were treated with anti-HER2-TLR7 conjugate or unconjugated HER2 mAb SC at 5 mg/kg and 20 mg/kg. Mice that had completely cleared tumors with HER2-TLR7 treatment were re-challenged with the same HER2 positive CT26 cell line approximately sixty days after primary tumor clearance. The half-life of the surrogate is approximately 48 hours and is no longer present at the time of re-challenge. anti-HER2-TLR7 conjugate treated mice for re-challenge were obtained as follows. BALB/cJ mice were inoculated subcutaneously in the right flank with 5×10⁵ HER2+CT26 cells in PBS. Fourteen days later, tumors were measured with calipers and volume was calculated using the formula volume=((minimum length)2×(maximum length))/2. Mice were sorted into control and treatment cohorts with size matched tumors. The mice were treated with PBS, 5 mg/kg anti-HER2 antibody, 5 mg/kg anti-HER2-TLR7 conjugate (Compound 2.1), 20 mg/kg anti-HER antibody or 20 mg/kg anti-HER2-TLR7 conjugate. Some 5 mg/kg and 20 mg/kg conjugate treated animals cleared their tumor, 25% and 30% respectively, while there were no clearances in the unconjugated HER2 antibody or PBS groups. Those exhibiting complete clearance were re-challenged with 5×10⁵ HER2+CT26 cells injected into the left flank along with cohorts of naïve animals that were similarly challenged. Mice re-challenged with HER2 positive CT26 tumors were 100% protected, indicating that the anti-HER2-TLR7 conjugate can induce a durable anti-tumor memory response at 5 mg/kg (FIG. 7A) and 20 mg/kg (FIG. 7B).

### Example 11

### Mice that have Cleared HER2+CT26 Tumors in Response to Anti-HER2-TLR7 Conjugate Reject HER2neg CT26 Tumors Upon Re-Challenge

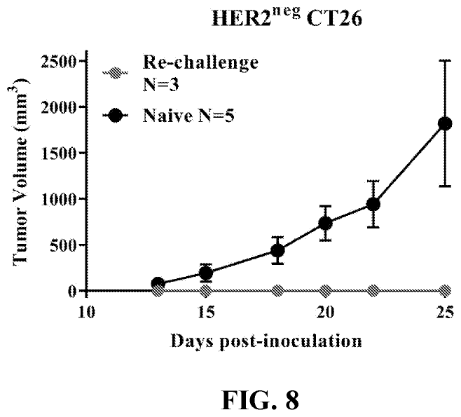

To test the durability and epitope spreading of the anti-tumor responses in mice treated with HER2-TLR7 conjugate mice that had completely cleared tumors with anti-HER2-TLR7 conjugate treatment were re-challenged with wild-type (HER2 negative) CT26 cells in the left flank sixty days after primary tumor clearance. The mice for re-challenge were obtained as follows. Female BALB/cJ mice were inoculated SC in the right flank with 5×10⁵ HER2+CT26 cells in PBS. Fourteen days later, tumors were measured with calipers and volume was calculated using the formula volume=((minimum length)2×(maximum length))/2. Mice with tumor volumes ranging from 96.5-146.3 mice were organized into 2 groups of 10 with average tumor size 126.8 mm³. Cohorts of 10 mice were treated SC with PBS or 50 mg/kg anti-HER2-TLR7 conjugate (Compound 2.1) qW×4. The anti-HER2-TLR7 conjugate treated mice that were tumor-free (30%) were then inoculated SC after approximately 60 days on the left flank with 5×10⁶ HER2-negative CT26 cells. As a control a cohort of naïve BALB/cJ mice were similarly inoculated with HER2-negative CT26 cells. Unlike the naïve controls, all the re-challenged mice were protected from growth of wild-type CT26 tumor cells, indicating a durable and broad neo-antigen T cells response that is independent of HER2 (FIG. 8).

### Example 12

### Anti-HER2-TLR7 Conjugate Induces TNF-α from Mouse Bone Marrow Derived Macrophages in the Presence of HER2pos Cells

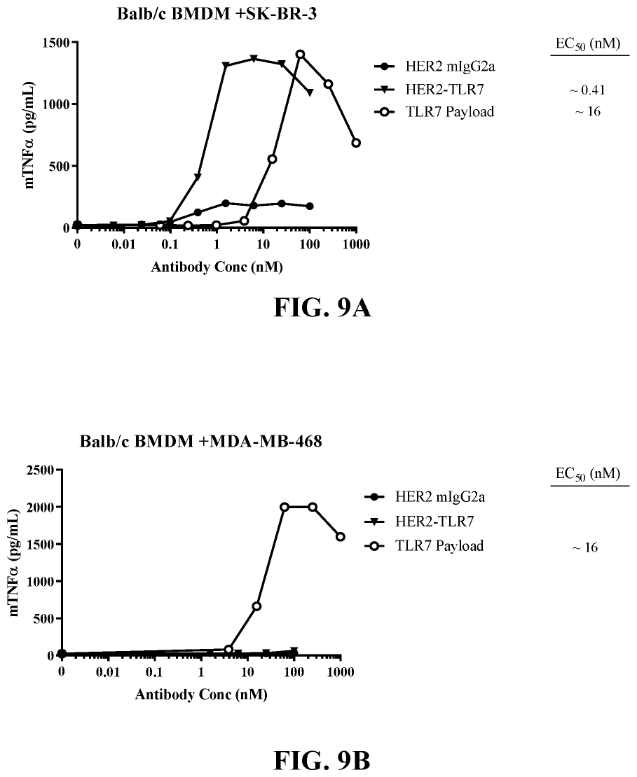

The ability of an anti-HER2-TLR7 conjugate to specifically activate mouse macrophages when bound to tumor cells by HER2 was assessed in vitro as follows. Bone marrow cells were harvested from BALB/cJ mouse femurs and tibias using a 27-gauge needle attached to a 3 mL syringe filled with growth media (DMEM supplemented with 10% Fetal Bovine Serum, 1 mM Sodium Pyruvate, 1× GlutaMAX-1, 1× Non-Essential Amino Acids, 10 mM HEPES and 0.5% Penicillin/Streptomycin). Bone marrow cells were centrifuged, and RBC lysed before being counted and resuspended at a concentration 5×10⁵/mL in growth media. Ten mL of cell suspension was placed in 10 cm dishes and 20 ng/mL murine macrophage-colony-stimulating factor (mM-CSF) was added. Cells were incubated for two days, media replaced with fresh growth media containing 20 ng/mL mM-CSF, and then cultured for a further four days. BMDM and tumor cell lines SK-BR-3 (HER2pos) or MDA-MB-468 (HER2neg) were removed from plates with Accutase cell detachment solution and counted. BMDM were plated in 96-well flat bottom microtiter plates at 80,000 cells/well in assay media (RPMI-1640 Medium supplemented with 10% Fetal Bovine Serum, 1 mM Sodium Pyruvate, 1× GlutaMAX-1, 1× non-essential Amino Acids, 10 mM HEPES and 0.5% Penicillin/Streptomycin). Tumor cell lines were plated at 40,000 cells/well in assay media along with 100-0.001 nM anti-HER2-TLR7 conjugate (Compound 2.1) or anti-HER2 mIgG2a or 1000-0.001 nM Compound 1.2 (TLR7 payload) and incubated together for 24 hours at 37° C., 5% CO₂. After culture, supernatants were collected and frozen at −80° C. until cytokine analysis was performed. Murine TNFα (mTNFα) levels in the supernatant were determined by mTNFα ELISA Kit (BioLegend) and read on an Envision Plate Reader (Perkin Elmer, Waltham, Mass.) according to manufactures instructions. mTNFα levels were then graphed using GraphPad Prism 7.01 software (GraphPad Software, San Diego, Calif.) and EC50 values generated using non-linear regression curve fit. The anti-HER2-TLR7 conjugate potently activated the mouse bone marrow cells when bound to the HER2pos cell line (FIG. 9A) but not when unbound in the presence of the HER2neg cell line (FIG. 9B). The TLR7 small molecule was capable of potently activating the macrophages in the presence of both cell lines.

### Example 13

### Elevated Intratumoral Cytokines, Chemokines, and Infiltration/Activation of Immune Cells in HER2+CT26 Tumor Bearing Mice after Treatment with Anti-HER2-TLR7 Conjugate

To demonstrate the ability of tumor targeted TLR7 immune activation mice bearing HER2+ tumors were treated with an anti-HER2-TLR7 conjugate (Compound 2.1) or anti-HER2 antibody control, tumors were excised and analyzed for immune activation by measuring immune cells, cytokines and chemokines. Six to eight-week-old BALB/cJ mice were inoculated SC in the right flank with 5×10⁵ HER2+CT26 cells. Seventeen days later, tumors were measured with calipers and volume was calculated using the formula volume=((minimum length)²×(maximum length))/2. Mice with tumor volumes ranging from 120.4-314.9 mm³ mice were organized into 4 groups of 6-7 with average tumor size 213.2 mm³. Mice were administered HER2 mAb or anti-HER2-TLR7 conjugate IV at 5 mg/kg and tumors were harvested on the schedule outlined in the Table. Intratumoral cytokines and chemokines were assayed by Luminex, and infiltrating immune cells were assessed by flow cytometry as follows. For Luminex analysis, tumors were weighed, placed into 500 uL RPMI and mechanically dissociated on ice. The resulting supernatants were stored at −80C for future analysis. Data was expressed as picogram of analyte per gram of starting tissue. A subset of tumors was also enzymatically digested using the Miltenyi mouse digest kit and filtered through a 70 um filter. Single cell suspensions were divided across 3 flow cytometry panels. For intracellular T cell analysis, cells were stimulated with 2 μM AH-1 peptide (AnaSpec (AS-64798)) in the presence of 1× brefeldin A for 4 hours at 37C, stained for surface markers, permeabilized with FoxP3 Staining Buffer (eBioscience), and stained with antibodies against TNFα and IFNγ. All data was analyzed in GraphPad Prism. *Note, in some cases anti-HER2-TLR7 conjugate-treated tumor material was limiting and was not available for all analyses.

| Group | Test Materials | Dose, Route and Schedule | N |
|---|---|---|---|
| A | HER2 mAb | 5 mg/kg, IV, 1x, | 7 |
| | | harvest at 48 h | |
| B | HER2-TLR7 | 5 mg/kg, IV, 1x, | 6-7* |
| | | harvest at 48 h | |
| C | HER2 mAb | 5 mg/kg, IV, 3x, days 0, 2, 4. | 6 |
| | | Harvest day 6 | |
| | | (48 hours post-dose #3) | |
| D | HER2-TLR7 | 5 mg/kg, IV, 3x, days 0, 2, 4. | 5-6* |
| | | Harvest day 6 | |
| | | (48 hours post-dose #3) | |

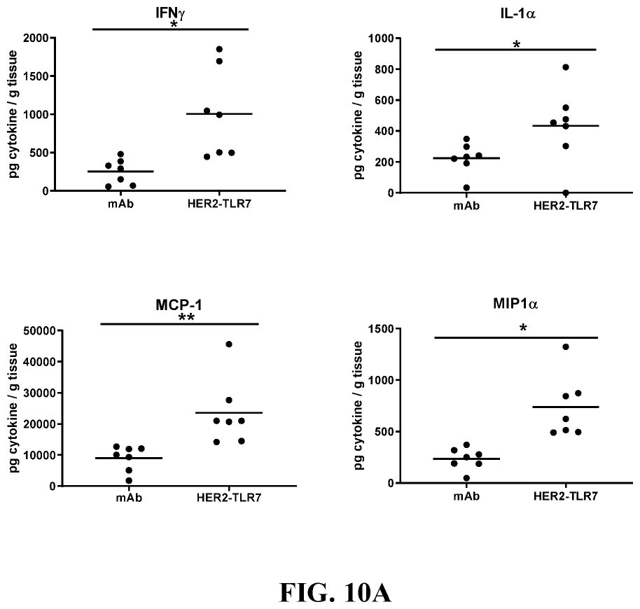

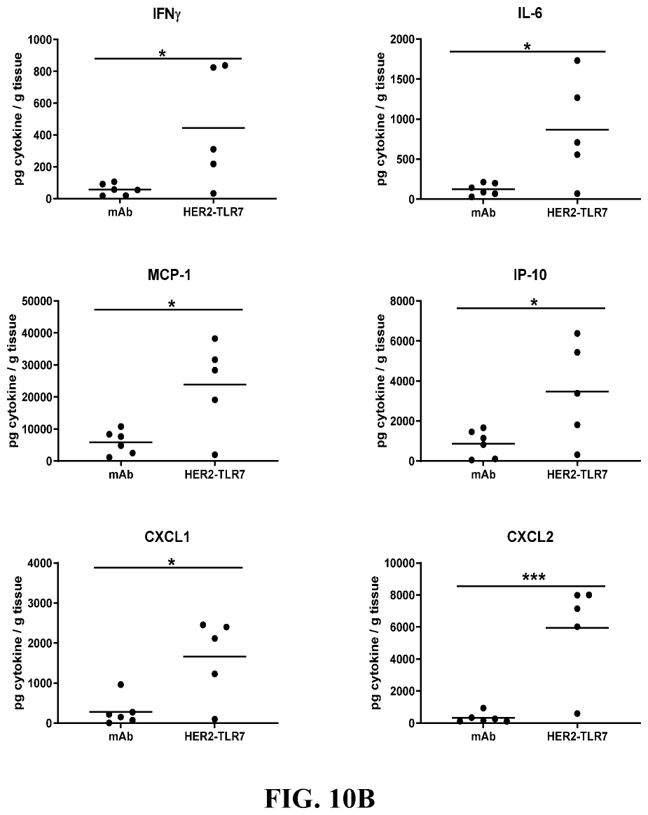

Compared to controls, intratumoral levels of indicated chemokines and cytokines were found to be elevated 48 hours post a single dose (FIG. 10A) or 3 doses (FIG. 10B) of the anti-HER2-TLR7 conjugate indicating increased immune activation. Statistical significance was determined by unpaired T-test. *p<0.05, **p<0.01, p<0.001

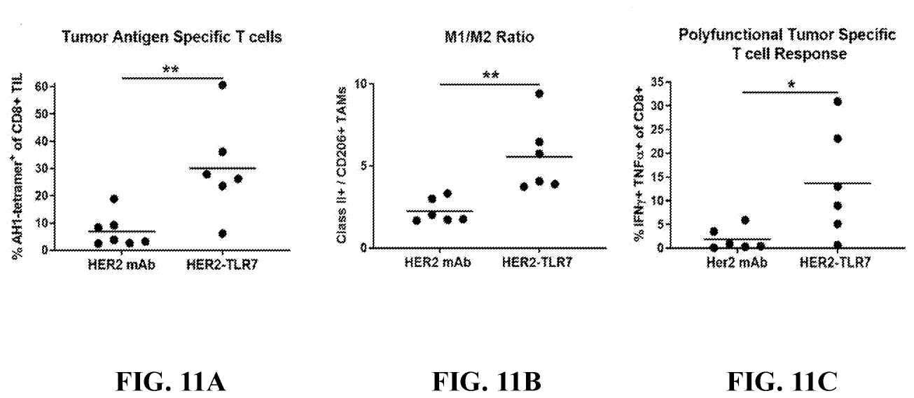

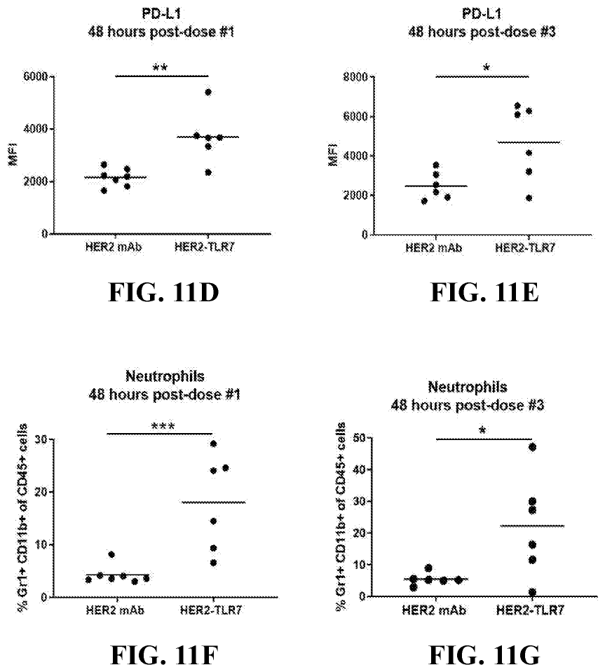

Compared to controls FACS analysis indicated intratumoral innate and adaptive immune cell activation was increased 48 hours post a single dose or three doses (day 6). By 48 hours, an expanded AH-1+ tumor antigen T cell population was identified by tetramer staining (FIG. 11A). At day 6 there was an increase in the macrophage M1 to M2 ratio (MHC Class II+:CD206+) (FIG. 11B) and an expansion of AH-1 responsive CD8 T cells (FIG. 11C). Elevated tumor cell surface PD-L1 expression (FIG. 11D-FIG. 11E) and neutrophil infiltrate (FIG. 11F-FIG. 11G) were observed at both timepoints. Statistical significance was determined by unpaired T-test. *p<0.05, **p<0.01, p<0.001.

Together these data indicate that treatment with the anti-HER2-TLR7 conjugate increased broad intratumoral immune activation.

1. A compound represented by Formula (IA): 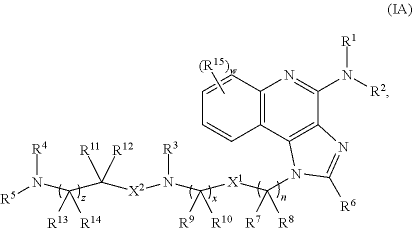
or a salt thereof, wherein:
R¹, R², R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R⁷, R¹, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;
R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;
R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
X¹ is O, S, or NR¹⁶;
X² is C(O) or S(O)₂;
n is 1, 2, or 3;
x is 1, 2, or 3;
w is 0, 1, 2, 3, or 4; and
z is 0, 1, or 2.

2.-34. (canceled)

35. A compound represented by Formula (IIA): 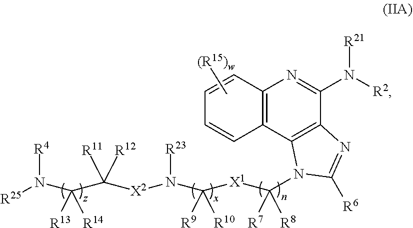
or a salt thereof, wherein:
R² and R⁴ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²¹, R²³, and R²⁵ is L³;
R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R⁷, R¹, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;
R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, —CN, C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;
R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
L³ is a linker;
X¹ is O, S, or NR¹⁶;
X² is C(O) or S(O)₂;
n is 1, 2, or 3;
x is 1, 2, or 3;
w is 0, 1, 2, 3, or 4; and
z is 0, 1, or 2,
wherein:
a) L³ is represented b the formula: 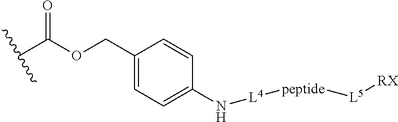
wherein:
L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and
R³⁰ is independently selected at each occurrence from halogen, —OH, —CN, —O-alkyl, —SH, ═O, ═S, —NH₂, —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂; or
b) L³ is represented by the formula: 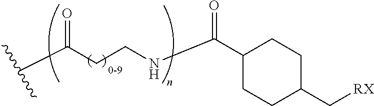
wherein:
RX comprises a reactive moiety; and
n is 0-9.

36.-40. (canceled)

41. The compound or salt of claim 35, wherein the compound of Formula (IIA) is represented by (IIB) or (IIC): 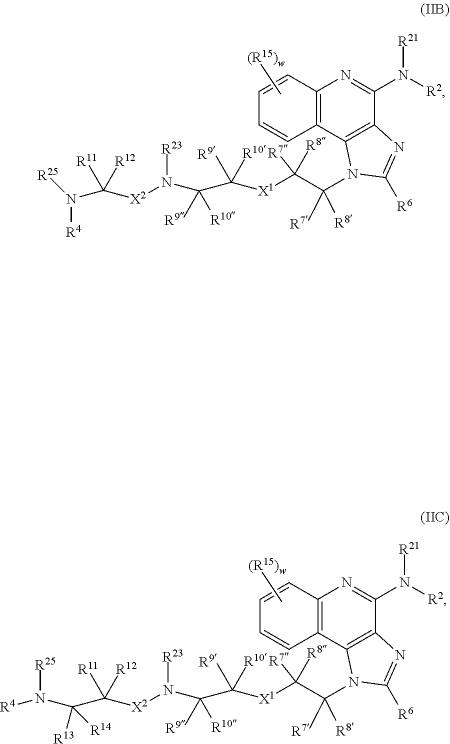
or a salt thereof, wherein:
R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

42. The compound or salt of claim 35, wherein R² and R⁴ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN.

43. The compound or salt of claim 42, wherein R² and R⁴ are independently selected from hydrogen and C₁₋₆ alkyl.

44. (canceled)

45. The compound or salt of claim 35, wherein R²³ is selected from hydrogen and C₁₋₆ alkyl optionally substituted with one or more halogens.

46. (canceled)

47. The compound or salt of claim 35, wherein R²¹ is selected from hydrogen and C₁₋₆ alkyl optionally substituted with one or more halogens.

48. (canceled)

49. (canceled)

50. The compound or salt of claim 35, wherein R²⁵ is selected from hydrogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN.

51.-53. (canceled)

54. The compound or salt of claim 35, wherein R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and
R²⁰ is independently selected at each occurrence from hydrogen, —NH₂, —C(O)OCH₂C₆H₅; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle.

55. The compound or salt of claim 35, wherein R⁶ is C₁₋₆ alkyl substituted with —OR²⁰, and
R²⁰ is selected from hydrogen and C₁₋₆ alkyl, which is optionally substituted with one or more substituents independently selected from halogen, —OH, and —NH₂.

56. The compound or salt of claim 41, wherein R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl optionally substituted with one or more substituents independently selected from halogen.

57.-59. (canceled)

60. The compound or salt of claim 56, wherein R^{9′}, R^{9″}, R^{10′}, and R^{10″} are independently selected at each occurrence from hydrogen and C₁₋₆ alkyl.

61. (canceled)

62. The compound or salt of claim 35, wherein R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

63. (canceled)

64. (canceled)

65. The compound or salt of claim 35, wherein R¹¹ and R¹² taken together form an optionally substituted C₃₋₆ carbocycle.

66. The compound or salt of claim 35, wherein X² is C(O).

67.-69. (canceled)

70. The compound or salt of claim 35, wherein RX comprises a leaving group.

71. The compound or salt of claim 35, wherein RX is a maleimide or an alpha-halo carbonyl.

72. The compound or salt of claim 35, wherein L³ is represented by the structure defined in claim 35 (a) and the peptide of L³ comprises Val-Cit or Val-Ala.

73.-75. (canceled)

76. The compound or salt of claim 35, wherein L³ is further covalently bound to an antibody construct to form a conjugate.

77. A conjugate represented by the formula: 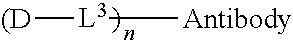
wherein:
Antibody is an antibody construct;
n is 1 to 20;
D is the compound or salt of claim 1; and
L³ is a linker moiety.

78.-84. (canceled)

85. A pharmaceutical composition, comprising a conjugate of claim 76, and a pharmaceutically acceptable excipient.

86. (canceled)

87. A method for the treatment of cancer, comprising administering an effective amount of the compound or salt of claim 1 to a subject in need thereof.

88. A method for the treatment of cancer, comprising administering an effective amount of the conjugate of claim 77 to a subject in need thereof.

89. (canceled)

90. A method for treatment, comprising administering to a subject the pharmaceutical composition of claim 85.

91. A method for the treatment of cancer, comprising administering to a subject in need thereof the pharmaceutical composition of claim 85.

92.-99. (canceled)
